SEQUENCE LISTING (2 sequences)

SEQ ID NO: 1 - DNA, 30 bp, Artificial Sequence
  Synthetic
aagcagtggtatcaacgcagagtacatggg

SEQ ID NO: 2 - DNA, 30 bp, Artificial Sequence
  Synthetic
ttcgtcaccatagttgcgtctcatgtaccc

(19) United States
(12) Patent Application Publication
(10) Pub. No.: US 2022/0251660 A1
(43) Pub. Date: Aug. 11, 2022

(54) DIAGNOSIS AND TREATMENT OF CARDIOMYOPATHY

(71) Applicants: The Broad Institute, Inc., Cambridge, MA (US); Bayer Aktiengesellschaft, Leverkusen (DE); The General Hospital Corporation, Boston, MA (US)

(72) Inventors: Patrick T. Ellinor, Cambridge, MA (US); Mark Chaffin, Cambridge, MA (US); Amer-Denis Akkad, Brookline, MA (US); Christian Stegmann, Wallisellen (CH); Irinna Papangeli, Larkspur, CA (US)

(21) Appl. No.: 17/669,904

(22) Filed: Feb. 11, 2022

Related U.S. Application Data
(60) Provisional application No. 63/254,964, filed on Oct. 12, 2021.
(60) Provisional application No. 63/148,750, filed on Feb. 12, 2021.

(30) Foreign Application Priority Data
Feb. 11, 2021 (GR) 20210100092

(51) Int. Cl. C12Q 1/6883; G01N 33/68; A61K 35/33

(52) U.S. Cl. CPC C12Q 1/6883; C12Q 1/6869; A61K 35/33; G01N 33/6893

(57) ABSTRACT

Provided herein are methods for diagnosing cardiomyopathy by evaluating a sample from a subject for the presence of a population of activated fibroblasts comprising a particular genetic signature (e.g., a genetic signature comprised of differential expression of one or more gene products relative to a normal subject or a population of normal subjects). The one or more gene products may include POSTN, COL22A1, and/or THBS4. Also provided herein are methods for treating cardiomyopathy and for modulating the activity of one or more gene products associated with activation of cardiac fibroblasts in a subject using an agent. Compositions comprising an agent for treating cardiomyopathy and kits for diagnosing cardiomyopathy are also provided by the present disclosure.

### RELATED APPLICATIONS

This application claims priority under 35 U.S.C. § 119(e) to U.S. Provisional application, U.S. Ser. No. 63/254,964, filed Oct. 12, 2021, and U.S. Provisional application, U.S. Ser. No. 63/148,750, filed Feb. 12, 2021, which claims priority under 35 U.S.C. § 119(a) to Greek Patent Application No. 20210100092, filed Feb. 11, 2021, each of which is incorporated herein by reference.

### GOVERNMENT SUPPORT

This invention was made with government support under Grant Nos. HL092577, HL128914, HL105780, HL140187, HL105993, and HL007208 awarded by the National Institutes of Health. The government has certain rights in the invention.

### REFERENCE TO SEQUENCE LISTING

The present application contains a Sequence Listing which has been submitted in ASCII format via EFS-Web and is hereby incorporated by reference in its entirety. Said ASCII copy, created on Feb. 7, 2022, and named B119570119US02-SEQ-TNG.txt, is 755 bytes in size.

### BACKGROUND OF THE INVENTION

Heart failure (HF) is a growing public health concern, with an estimated worldwide prevalence of more than 26 million people.¹ HF encompasses a heterogenous set of clinical features, converging on impaired cardiac contractile function.² Understanding the underlying mechanisms of HF has been at the forefront of cardiovascular research. Further work has been needed to elucidate these mechanisms and exploit them for diagnosis and treatment of diseases that lead to HF, such as cardiomyopathy. Cardiomyopathy is a group of diseases that affect the heart muscle. There is often a genetic basis for the development of cardiomyopathy, and in many cases, it is inherited within families. In other cases, causes of cardiomyopathy may include, but are not limited to, viral infections, coronary heart disease, drug use, alcohol use, or exposure to heavy metals. In many cases, the causes of cardiomyopathy are unknown, and the molecular mechanisms involved in cardiomyopathy are still poorly understood, limiting treatment options.

### SUMMARY OF THE INVENTION

Historically, cardiomyocytes have been the primary research focus in HF due to their role in cardiac contraction; however, in recent years focus has shifted to other cardiac cell types, including vascular, interstitial, and neuronal cells.³ These efforts have been greatly augmented by the recent advances in single cell and single nucleus sequencing technologies, which allow for transcriptomic analysis at the single cell level.⁴⁻⁶ The present disclosure describes the identification of transcriptional alterations present in HF by performing single-nuclei RNA sequencing (snRNA-seq) of left ventricle biopsies from patients with dilated cardiomyopathy (DCM), hypertrophic cardiomyopathy (HCM), and non-failing (NF) hearts. Nearly 600,000 nuclei were captured and classified into 21 clusters. By comparing gene expression between cardiomyopathy and NF patients, a convergence of transcriptional profiles of DCM and HCM patients was observed. Further, a population of proliferating macrophages that were reduced in cardiomyopathy patients and a cardiomyopathy-specific population of activated fibroblasts nearly entirely absent from NF samples were discovered. Through application of a deconvolution method to the bulk RNA sequencing data, enrichment of this activated fibroblast population in an independent set of cardiomyopathy patients was confirmed. The present disclosure therefore provides novel insights into the transcriptional diversity at single cell resolution for the human heart in health and disease, particularly cardiomyopathy. The genetic signatures associated with cardiomyopathy disclosed herein may be useful for diagnosing and treating patients with cardiomyopathy.

Accordingly, the present disclosure provides methods for diagnosing cardiomyopathy in a subject. Also, disclosed herein are methods for treating cardiomyopathy in a subject and methods of modulating the expression of gene products associated with activation of cardiac fibroblasts in a subject. Further disclosed herein are compositions comprising an agent capable of modulating the expression of gene products associated with the activation of cardiac fibroblasts. The present disclosure also provides kits comprising reagents for performing the methods disclosed herein.

In one aspect, the present disclosure provides methods of diagnosing cardiomyopathy (e.g., dilated cardiomyopathy or hypertrophic cardiomyopathy) in a subject comprising (a) providing a sample from a subject (for example, a biopsy from the heart, or a blood sample); and (b) evaluating the sample for the presence of a population of activated fibroblasts comprising a specific genetic signature. The genetic signature may comprise increased or decreased expression (e.g., relative to a sample provided from a normal subject or a population of normal subjects) of one or more gene products selected from the group consisting of periostin (POSTN), NADPH Oxidase 4 (NOX4), fibroblast activation protein (FAP), collagen type I alpha 1 chain (COL1A1), collagen type I alpha 2 chain (COL1A2), actin alpha 2 (ACTA2), solute carrier family 44 member 5 (SLC44A5), collagen type XXII alpha 1 chain (COL22A1), Ae binding protein 1 (AEBP1), thrombospondin-4 (THBS4), family with sequence similarity 155, member A (FAM155A), teashirt zinc finger homeobox 2 (TSHZ2), juxtaposed with another zinc finger protein 1 (JAZF1), proline and arginine rich end leucine rich repeat protein (PRELP), calsyntenin 2 (CLSTN2), integrin alpha 10 (ITGA10), cell adhesion molecule 1 (CADM1), neuronal growth regulator 1 (NEGR1), platelet-derived growth factor receptor A (PDGFRA), complement component 7 (C7), fibulin 5 (FBLN5), and collagen type IV alpha 4 chain (COL4A4). In some embodiments, the genetic signature comprises increased or decreased expression (e.g., relative to a sample provided from a normal subject or a population of normal subjects) of one or more gene products selected from the group consisting of periostin (POSTN), NADPH Oxidase 4 (NOX4), fibroblast activation protein (FAP), collagen type I alpha 1 chain (COLlAl), collagen type I alpha 2 chain (COL1A2), actin alpha 2 (ACTA2), solute carrier family 44 member 5 (SLC44A5), collagen type XXII alpha 1 chain (COL22A1), Ae binding protein 1 (AEBP1), thrombospondin-4 (THBS4), family with sequence similarity 155, member A (FAM155A), teashirt zinc finger homeobox 2 (TSHZ2), neuronal growth regulator 1 (NEGR1), platelet-derived growth factor receptor A (PDGFRA), complement component 7 (C7), fibulin 5 (FBLN5), and collagen type IV alpha 4 chain (COL4A4). In some embodiments, a genetic signature comprises increased expression of COL22A1. In certain embodiments, a genetic signature comprises increased expression of POSTN. In certain embodiments, a genetic signature comprises increased expression of THBS4. In some embodiments, a genetic signature comprises increased expression of COL22A1, POSTN, and THBS4. In certain embodiments, the genetic signature comprises increased or decreased expression of one or more genes selected from the group consisting of NEGR1, FBLN5, PRELP, CLSTN2, ITGA10, JAZF1, COL22A1, AEBP1, FGF14, THBS4, FAP, and CADM1.

In another aspect, the present disclosure provides methods of treating cardiomyopathy (e.g., dilated cardiomyopathy or hypertrophic cardiomyopathy) in a subject in need thereof. In some embodiments, the methods of treatment comprise administering a treatment to the subject based on the presence of a population of activated fibroblasts comprising a genetic signature, as described herein. The use of any treatment for cardiomyopathy known in the art is contemplated, including exercise, surgery, use of a medical device, and/or pharmacological intervention. In some embodiments, a treatment comprises lifestyle changes (e.g., exercise), use of a pacemaker, use of a defibrillator, use of a ventricular assist device, ablation, an angiotensin-converting enzyme (ACE) inhibitor, an angiotensin II receptor blocker, a beta blocker, a diuretic, digoxin, a blood-thinning medication, or a heart transplant. In some embodiments, the methods of treatment comprise administering to a subject a therapeutically effective amount of an agent (e.g., a small molecule, a nucleic acid, such as an siRNA, or a protein, such as an antibody) capable of modulating the activity of one or more gene products associated with activation of cardiac fibroblasts, wherein the one or more gene products is selected from the group consisting of POSTN, NOX4, FAP, COL1A1, COL1A2, ACTA2, SLC44A5, COL22A1, AEBP1, THBS4, FAM155A, TSHZ2, JAZF1, PRELP, CLSTN2, ITGA10, cell adhesion molecule 1 (CADM1), NEGR1, PDGFRA, C7, FBLN5, and COL4A4. In certain embodiments, the genetic signature comprises increased or decreased expression of one or more genes selected from the group consisting of NEGR1, FBLN5, PRELP, CLSTN2, ITGA10, JAZF1, COL22A1, AEBP1, FGF14, THBS4, FAP, and CADM1. In some embodiments, the methods of treatment comprise administering to a subject a therapeutically effective amount of an agent (e.g., a small molecule, a nucleic acid such as an siRNA, or a protein such as an antibody) capable of modulating the activity of one or more gene products associated with activation of cardiac fibroblasts, wherein the one or more gene products is selected from the group consisting of POSTN, NOX4, FAP, COL1A1, COL1A2, ACTA2, SLC44A5, COL22A1, AEBP1, THBS4, FAM155A, TSHZ2, NEGR1, PDGFRA, C7, FBLN5, and COL4A4. In another aspect, methods for modulating the activity of any of the gene products associated with activation of cardiac fibroblasts described herein with an agent are provided by the present disclosure. In some embodiments, the activity of COL22A1 is modulated. In some embodiments, the activity of POSTN is modulated. In certain embodiments, the activity of THBS4 is modulated.

In another aspect, provided herein are compositions comprising an agent (e.g., a small molecule, a nucleic acid such as an siRNA, or a protein such as an antibody) capable of modulating the activity of one or more gene products associated with activated fibroblasts. In some embodiments, the composition also comprises a pharmaceutically acceptable excipient.

In another aspect, kits for diagnosing a subject having, suspected of having, or at risk of having cardiomyopathy are also provided herein. In some embodiments, the kits comprise reagents for performing any of the methods disclosed herein.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1F provide an overview of cellular composition in the left ventricles from healthy patients and those with cardiomyopathy. FIG. 1A shows uniform manifold approximation and projection (UMAP) representation of 592,689 nuclei isolated from the left ventricles of 42 patients. Each dot represents a nucleus, shaded and labeled by the cluster identified using unsupervised Leiden clustering. FIG. 1B shows dendrogram demonstrating similarity of cluster centroids based on the mean expression of the top 2000 most highly variable genes using Euclidean distance and Ward linkage. Each cell type is shaded and labeled to match FIG. 1A. FIG. 1C shows a stacked bar plot depicting the cell type composition of each sample with shading reflecting cell types in FIG. 1B. Each bar sums to a total of one and the relative contribution of each cell type from FIG. 1A is represented by its respective shade. FIG. 1D shows a number of deleterious variants (loss-of-function or pathogenic/likely pathogenic in ClinVar) identified in carriers of known clinical cardiomyopathy testing panel genes by disease state. FIG. 1E shows principal component analysis on pseudo-bulk single-nuclei RNA sequencing (top 500 most variable genes) by disease status and sex. The percent variance captured by each principal component is shown in parentheses on each respective axis. DCM=Dilated cardiomyopathy, HCM=Hypertrophic cardiomyopathy, HCMrEF=Hypertrophic cardiomyopathy with reduced ejection fraction, HCMpEF=Hypertrophic cardiomyopathy with preserved ejection fraction, VSMC=Vascular smooth muscle cell. FIG. 1F shows principal component analysis of pseudo-bulk single-nuclei RNA sequencing by disease status and sex. The percent variance captured by each principal component is shown in parentheses on each respective axis.

FIGS. 2A-2E show transcriptional differences between non-failing and cardiomyopathy left ventricles. FIG. 2A shows volcano plots displaying the log fold-change and p-value for expression changes between dilated cardiomyopathy (DCM) and non-failing (NF) (left), hypertrophic cardiomyopathy (HCM) and NF (center), and HCM and DCM (right) based on CellBender remove-background counts. Genes are shaded by cell type with larger, opaque dots representing genes with FDR<0.01. Only genes deemed to have a low probability of background contamination are displayed. FIG. 2B shows a number of significantly differentially expressed genes (FDR<0.01 for both CellBender remove-background and CellRanger counts) by cell type for each disease comparison in FIG. 2A. FIG. 2C shows spearman correlation of log fold-change estimates from snRNA-seq and bulk RNA sequencing for each comparison (y-axis) within each cell type (x-axis). Only overlapping samples (11 DCM, 10 HCM, and 13 NF) are in each respective differential expression test. FIG. 2D shows reactome pathway enrichment for differential expression between DCM vs. NF (left) and HCM vs. NF (right) by cell type. The size of the square reflects the P-value from a gene set enrichment test, and the shade represents the normalized enrichment score. Any pathway significant with FDR<0.05 is denoted with a dot. log(FC)=log fold-change, DCM=Dilated cardiomyopathy, HCM=Hypertrophic cardiomyopathy, NF=Non-failing, ρ=Spearman correlation, NES=Normalized enrichment score, VSMC=Vascular smooth muscle cell. Pint, P-value for interaction between cardiomyopathy and sex. FIG. 2E shows log₂ counts per million (CPM) across samples for genes with a significant disease-sex interaction in either DCM or HCM (FDR<0.1).

FIGS. 3A-3D show sub-populations of macrophages distributed differentially by disease state. FIG. 3A shows uniform manifold approximation and projection (UMAP) representation of 53,730 nuclei classified as macrophage or proliferating macrophage in the global analysis. Each dot represents a nucleus, shaded and labeled by the cluster identified using unsupervised Leiden clustering. FIG. 3B shows distribution of macrophage sub-clusters across patients by disease status. FIG. 3C provides a dendrogram demonstrating similarity of sub-cluster centroids based on the mean expression of the top 2000 most highly variable genes using Euclidean distance and Ward linkage. FIG. 3D shows expression patterns of markers for each sub-cluster compared to all other sub-clusters. Dot size indicates the percent of nuclei expressing the gene at non-zero levels in the given sub-cluster and the shade represents the average normalized expression, scaled to the max expression for each gene across all sub-clusters. Mcp=Macrophage, NF=Non-failing, DCM=Dilated cardiomyopathy, HCM=Hypertrophic cardiomyopathy, Pct Nuclei Expr >0=Percent of nuclei expressing the gene at non-zero levels, Avg Norm Expr-Average normalized expression, scaled to the max expression for each gene across all sub-clusters.

FIGS. 4A-4I show discovery of a DCM- and HCM-specific activated fibroblast population. FIG. 4A shows the number of activated fibroblasts per disease state by patient. Patients with more than 100 nuclei classified as activated fibroblasts are labeled. FIG. 4B provides a dot plot displaying the expression patterns of markers for activated fibroblasts. Dot size indicates the percent of nuclei expressing the gene at non-zero levels in the given sub-cluster and the shade represents the average log-normalized expression calculated as log1p(gene counts/total counts*10000). FIG. 4C shows RNA in situ hybridization of activated fibroblasts in four patients. Probes for DCN, a marker of general fibroblasts, and COL22A1, a marker of activated fibroblasts, were used. Localization of nuclei is shown with hematoxylin stain. FIG. 4D shows uniform manifold and projection (UMAP) representation of all fibroblasts from two patients with the largest activated fibroblast populations. Connectivity induced by the minimum spanning tree from Slingshot is overlaid. FIG. 4E shows uniform manifold and projection (UMAP) representation of all fibroblasts from patient P1304 (top) and P1425 (bottom) with all Slingshot inferred trajectories overlaid. The most representative quiescent to activated fibroblast trajectory is marked with an asterisk, and nuclei along this trajectory are shaded by estimated pseudo-time. Nuclei shaded grey are not considered to fall along the trajectory. FIG. 4F shows the UMAP representation with inferred RNA velocity overlaid as a streamplot. FIG. 4G shows negative binomial generalized additive model (NB-GAM) smoother for predicted expression of genes showing interesting patterns along inferred pseudo-time in both patients P1304 (left) and P1425 (right). Genes in the top show high expression in quiescent fibroblasts that decreases across the gradient toward activated fibroblasts, genes in the middle show elevated expression somewhere along the trajectory, and genes on the bottom show low expression in quiescent fibroblasts that increases across the gradient toward activated fibroblasts. FIG. 4H shows deconvolution analysis using CIBERSORTx of bulk RNA sequencing data from GEO accession number GSE141910 for overlapping patients with single-nuclei RNA sequencing (left) and non-overlapping (right). Each dot represents the estimated fraction of activated fibroblasts for a patient and the number of patients for each disease state is displayed under the respective x-axis label. DCM=Dilated cardiomyopathy, HCM=Hypertrophic cardiomyopathy, PPCM=Peripartum cardiomyopathy, NF=Non-failing, Avg Expr=natural log of (Gene counts/Total counts per nucleus*10000), Pct Nuclei Expr >0=Percent of nuclei expressing the gene at non-zero levels, VSMC=Vascular smooth muscle cell, Norm Expr=Smoothed expression from NB-GAM, scaled to the max value for each gene. FIG. 4I shows deconvolution of bulk RNA sequencing data from two external datasets displayed as in FIG. 4H. DCM, Dilated cardiomyopathy; HCM, Hypertrophic cardiomyopathy; PPCM, Peripartum cardiomyopathy; NF, Non-failing; Avg expr, Average log-normalized expression; Pct nuclei expr >0, Percent of nuclei expressing the gene at non-zero levels; Norm expr, Smoothed expression from NB-GAM scaled to the max value for each gene.

FIGS. 5A-5D show a cellular assay of myofibroblast transition in cardiac fibroblasts. FIG. 5A shows that a subset of genes from the activated fibroblast trajectory analysis (n=27) were knocked out in cardiac fibroblasts (CFs) using 2-4 sgRNA per gene, stimulated with TGFβ1, and assessed for changes in cellular phenotypes using high content imaging. CM, cardiomyocytes; NTC, non-targeting control. FIG. 5B shows a representative image of cardiac fibroblasts pre-TGFβ1 stimulation (left) and post-TGFβ1 stimulation (right) with non-targeting control sgRNA. FIG. 5C shows bar plots representing the mean fraction of myofibroblasts post-TGFβ1 stimulation across all wells for a given sgRNA, labeled by gene. Error bars represent standard error and * indicates that only 1 well was included after filtering, so no error bars are displayed. FIG. 5D shows representative images pre- and post-TGFβ1 stimulation for 3 control genes and 2 target genes showing strong effects.

FIGS. 6A-6C provide an assessment of sample quality control. FIG. 6A shows distribution of key quality control metrics from Cell Ranger count. Cutoffs for low quality are shown by the dashed line, and samples that fail on a given metric are labeled. Center line, median; box limits, upper and lower quartiles; whiskers, 1.5× interquartile range; points, outliers. FIG. 6B shows exemplar high-quality UMI decay curves (top row) and low-quality unique molecular identifier (UMI) decay curves (bottom row). Cell barcodes are ranked by the total UMI. FIG. 6C shows total counts from Y chromosome transcripts in phenotypically classified females (labeled females) and males (labeled males). NF=Non-failing, DCM=Dilated cardiomyopathy, HCM=Hypertrophic cardiomyopathy, UMI=Unique molecular identifier.

FIGS. 7A-7G provide an assessment of nuclei quality control. FIG. 7A shows uniform manifold and projection (UMAP) representation of all Cell Bender non-empty droplets (n=885,944), shaded and labeled by Leiden clustering at a resolution of 1.5. Labels are loosely based on selective gene expression in each cluster. FIG. 7B shows distribution of the mean of four quality control metrics across clusters in FIG. 7A, as boxplots, including percent of unique molecular identifiers mapping to mitochondrial genes (% MT), fraction of reads mapping exclusively to exons (Exonic Fraction), entropy, and the Scrublet estimated doublet score. Outlier clusters removed based on the criteria are shown as gray circles and labeled. Center line, median; box limits, upper and lower quartiles; whiskers, 1.5× interquartile range; points, outliers. FIG. 7C shows UMAP representation of non-empty droplets after removal of low-quality clusters identified in FIGS. 7A-7B. Additional low-quality nuclei as detected per cluster and per-sample are marked with asterisks. The distribution of number of unique molecular identifiers (nUMI), number of unique genes (nGene), % MT, and entropy across nuclei of each unique cell type is shown. Center line, median; box limits, upper and lower quartiles; whiskers, 1.5× interquartile range. FIG. 7D shows proportion of each cluster (top) and sample (bottom) removed (light gray) and retained (dark gray) during the quality control procedure. FIG. 7E shows UMAP representation of non-empty droplets after removal of low-quality clusters and per-cluster quality control. Black nuclei were deemed as misclassified or low-quality nuclei based on sub-cluster analysis within each cluster. FIG. 7F shows the average score (sc.tl.score_genes) for each Leiden cluster (x-axis) based on marker genes for each major cell type (y-axis). When a Leiden cluster scores highly for an unrelated cell type (black border), it was removed. CM=Cardiomyocyte, FB=Fibroblast, EC=Endothelial cell, PC: Pericyte, MP: Macrophage, VSMC: Vascular smooth muscle cell, LC1=Lymphocyte I, EndoC=Endocardial, AD=Adipocyte, NRN=Neuronal, LEC=Lymphatic endothelial, ActFB: Activated fibroblast, LC2=Lymphocyte II, ProfMP=Proliferating macrophages, EpiC: Epicardial, MT: Mitochondrial. FIG. 7G shows the distribution of number of unique molecular identifiers (nUMI), number of unique genes (nGene), % MT, and entropy across nuclei of each unique cell type. Center line, median; box limits, upper and lower quartiles; whiskers, 1.5× interquartile range.

FIGS. 8A-8H provide sample-level principal component analysis by cell type. Principal component analysis (PCA) using the top 500 most highly variables genes after summing expression counts for each sample, for pseudo-bulk (FIG. 8A), cardiomyocyte (FIG. 8B), fibroblast (FIG. 8C), endothelial cell (EC) I (FIG. 8D), pericyte (FIG. 8E), macrophage (FIG. 8F), vascular smooth muscle cell (VSMC) (FIG. 8G), and lymphocyte (FIG. 8H). Each dot represents a sample shaded and labeled by disease state with a shape to indicate sex, according to the legends provided. The analysis was run on both CellRanger and CellBender counts. The percent of total variation for each principal component (PC) is shown in parentheses. DCM=Dilated cardiomyopathy, HCMrEF=Hypertrophic cardiomyopathy with reduced ejection fraction, HCMpEF=Hypertrophic cardiomyopathy with preserved ejection fraction, VSMC=Vascular smooth muscle cell.

FIG. 9 shows overlap of differentially expressed genes using Cell Ranger counts versus Cell Bender counts. Venn diagrams showing the overlap of differentially expressed genes (FDR<0.01) for Cell Bender (light gray) versus Cell Ranger (dark gray). For each cell type, three (3) comparisons are shown: DCM vs NF, HCM vs NF, and DCM vs NF. The number of differentially expressed genes in each bin are labeled on the appropriate circles. DCM=Dilated cardiomyopathy, HCM=Hypertrophic cardiomyopathy, NF=Non-failing, VSMC=Vascular smooth muscle cell.

FIGS. 10A-10B show marker genes and cell type clustering. FIG. 10A provides dot plots showing the expression profile of the top 4 marker genes for each cell type. The size of the dot reflects the percent of nuclei expressing the gene at non-zero levels, and the shade reflects the mean log-normalized expression. FIG. 10B shows hierarchical clustering of cell types and expression profiles of the top 2000 most highly variable genes. Avg Expr, Average log-normalized expression; Pct Nuclei Expr >0, Percent of nuclei expressing the gene at non-zero levels.

FIGS. 11A-11C show whole genome sequencing analysis of cardiomyopathy patients. Volcano plots are provided displaying the log fold-change (log(FC)) and p-value for expression changes between dilated cardiomyopathy (DCM) TTN LOF carriers and DCM non-carriers (n=4 vs 6) (FIG. 11A), hypertrophic cardiomyopathy (HCM) MYBPC3 LOF carriers and HCM non-carriers (n=3 vs 12) (FIG. 11B), and HCM MYH7 pathogenic variant carriers and HCM non-carriers (n=5 vs 10) (FIG. 11C) based on CellBender remove-background counts. Dots are shaded by cell type with outlined dots representing genes with FDR<0.01. Only genes deemed to have a low probability of background contamination are displayed. VSMC, vascular smooth muscle cell.

FIGS. 12A-12F show sub-clustering of abundant cell types. Sub-clustering results for cardiomyocytes (FIG. 12A), fibroblasts (FIG. 12B), endothelial cells (FIG. 12C), pericytes (FIG. 12D), vascular smooth muscle cells (VSMC) (FIG. 12E), and lymphocytes (FIG. 12F) are shown. Uniform manifold approximation and projection (UMAP) visualization shaded by Leiden clusters is shown on the left. The distribution of sub-populations across patients by disease status represented as box plots with statistically credible changes indicated with a * (middle). Sub-population labels are shaded and labeled as in the UMAP visualization. Center line, median; box limits, upper and lower quartiles; whiskers, 1.5× interquartile range; points, outliers. Dot plots of the most selective markers for each sub-population compared to all other sub-populations (right). Avg norm expr, Average log-normalized expression scaled to the max value for each gene; Pct Nuclei Expr >0, Percent of nuclei expressing the gene at non-zero levels; Act. FB, Activated fibroblast; L-EC, Lymphatic endothelial.

FIGS. 13A-13I show macrophage sub-populations. FIG. 13A shows uniform manifold approximation and projection (UMAP) representation of 53,730 nuclei classified as macrophage or proliferating macrophage in the global analysis shaded and labeled by sub-population. FIG. 13B shows a UMAP plot with the S phase cell cycle score overlaid. FIG. 13C shows a UMAP plot with the G2M phase cell cycle score overlaid. FIG. 13D shows expression patterns of markers for each sub-population compared to all other sub-populations. FIG. 13E shows the distribution of macrophage sub-populations across patients by disease status represented as box plots, with statistically credible differences denoted with a *. Center line, median; box limits, upper and lower quartiles; whiskers, 1.5× interquartile range; points, outliers. FIG. 13F shows immunofluorescence staining for macrophage marker CD163, cycling marker MKI67, and nuclei with DAPI. FIG. 13G provides a dendrogram demonstrating similarity of sub-population centroids. FIGS. 13H-13I show expression of marker genes for CCR2 positive (CCR2+) (FIG. 13H) and CCR2 negative (CCR2−) (FIG. 13I) cardiac macrophages obtained from Bajpai et al., 2018.¹⁵ The size of each square represents the percent of nuclei expressing the given gene at non-zero levels in the given sub-population while the shade represents a formal log fold-change (log(FC)) estimate comparing the expression in the given sub-population to all other sub-populations. Boxes with a border indicate that the gene is significantly up- or down-regulated in the given sub-population with FDR<0.01. Mφ, Macrophage; NF, Non-failing; DCM, Dilated cardiomyopathy; HCM, Hypertrophic cardiomyopathy; Pct nuclei expr >0; Avg norm expr, Average normalized expression scaled to the max expression for each gene across all sub-populations.

FIGS. 14A-14C show single-nuclei RNA sequencing differential expression concordance with bulk RNA sequencing. FIG. 14A shows concordance of log fold-change (logFC) differential expression estimates based on single-nuclei RNA sequencing (snRNA-seq) pseudo-bulk (x-axis) compared to bulk RNA sequencing from an expanded set of samples in the MAGNet repository (GSE141910) including 166 dilated cardiomyopathy (DCM) patients, 27 hypertrophic cardiomyopathy (HCM) patients, and 162 non-failing (NF) patients (y-axis). Each dot represents a gene, and the comparison is shown for both Cell Ranger (top) and Cell Bender (bottom) results for DCM vs NF, HCM vs NF, and DCM vs HCM. FIG. 14B shows concordance of logFC differential expression estimates from snRNA-seq pseudo-bulk comparing DCM to NF with bulk RNA sequencing from Alimadadi et al., 2020.¹ Each dot represents a gene, and the comparison is shown for both Cell Ranger (top) and Cell Bender (bottom) results. FIG. 14C shows concordance of logFC differential expression estimates from snRNA-seq pseudo-bulk comparing HCM to NF with bulk RNA sequencing from Liu et al., 2019.² MAGNet=Myocardial Applied Genomics Network, DCM=Dilated cardiomyopathy, HCM=Hypertrophic cardiomyopathy, NF=Non-failing, logFC=log fold-change, snRNA-seq=single-nuclei RNA sequencing.

FIG. 15 shows CCR2 positive and CCR2 negative macrophage marker expression in single-nuclei RNA sequencing macrophage sub-clusters. Marker genes for CCR2 positive (CCR2+) and CCR2 negative (CCR2−) cardiac macrophages were obtained from Bajpai et al., 2018.3 Expression of each sub-cluster is shown for macrophage sub-clusters identified in single-nuclei RNA sequencing (snRNA-seq) analysis. Sub-clusters are shown on the x-axis and genes on the y-axis. The size of each square represents the percent of nuclei expressing the given gene at non-zero levels in the given cluster while the shade represents a formal log fold-change estimate comparing the expression in the given sub-cluster to all other sub-clusters. A dot indicates that the gene is significantly up- or down-regulated in the given cluster with FDR<0.01.

FIGS. 16A-16B show expanded in situ hybridization of two cardiomyopathy patients with activated fibroblasts and two non-failing patients without. FIG. 16A shows additional immunofluorescence staining of four patients for macrophage marker CD163 and cycling marker MKI67. Images are displayed both with, and without, DAPI to allow better visualizing of cells co-expressing CD163 and MKI67. FIG. 16B shows in situ hybridization with RNAscope showing localization of a canonical fibroblast marker, DCN, and an activated fibroblast marker, COL22A1, shaded according to the legend provided, across sections from dilated cardiomyopathy patient 1304, hypertrophic cardiomyopathy patient 1425, non-failing patient 1515, and non-failing patient 1516. Nuclear localization is shown with hematoxylin (shaded according to the legend provided). Images on the second row in each sample represent negative controls. NF, non-failing; DCM, dilated cardiomyopathy; HCM, hypertrophic cardiomyopathy.

FIG. 17 provides an overlay of mouse genes shown to change after mouse infarction injury with inferred fibroblast activation trajectory. Genes shown to have changes in gene expression over time following an infarction injury in mouse⁴ were grouped into 7 categories in the following order: cell migration, cytoskeleton, extracellular matrix, extracellular matrix modification, bone and cartilage, apoptosis inhibitor, and fibroblast marker. Negative binomial generalized additive model (NB-GAM) smoothers along inferred pseudo-time of the activated fibroblast trajectory in both P1304 (left) and P1425 (right) are shown. P-values reflect the tradeseq association test, which assesses whether there are significant changes in expression along the inferred activated fibroblast trajectory.

FIG. 18. shows activated fibroblast deconvolution of external DCM, ICM, HCM, and NF bulk RNA sequencing. Deconvolution analysis using CIBERSORTx of bulk RNA sequencing data from Gene Expression Omnibus (GEO) accession number GSE116250 containing DCM, ICM, and NF patients and GEO accession number GSE130036 containing HCM and NF individuals. Each dot represents the estimated fraction of activated fibroblasts for an individual and the number of patients for each disease state is displayed under the respective x-axis label. DCM=Dilated cardiomyopathy, ICM=Ischemic cardiomyopathy, HCM=Hypertrophic cardiomyopathy, NF=Non-failing.

FIGS. 19A-19D show validation of computational deconvolution analysis for activated fibroblasts. FIG. 19A shows in situ hybridization with RNAscope showing localization of a canonical fibroblast marker, DCN, and an activated fibroblast marker, COL22A1, both shaded according to the legend provided, across sections from six (6) patients. Nuclear localization is shown with hematoxylin (also shaded according to the legend provided). The computationally predicted percent of activated fibroblasts from CIBERSORTx (% Act. FB) for each patient is show above their respective image. FIGS. 19B-19C show uniform manifold and projection (UMAP) representation of all nuclei from both the primary analysis (n=42) and validation single-nuclei RNA-sequencing (n=3) (FIG. 19B), and separately for validation samples alone (FIG. 19C). The total number of samples (n_sample) and total nuclei (n_nuclei) are shown above each respective figure. FIG. 19D shows the relative contribution of fibroblasts and activated fibroblasts to each primary analysis patient (left) and validation patients only (right). DCM=Dilated cardiomyopathy, HCM=Hypertrophic cardiomyopathy, NF=Non-failing, VSMC=Vascular smooth muscle cell.

FIG. 20 shows replication of the cardiac fibroblast activation assay across up to five screens. The average percent change in the fraction of myofibroblasts across up to five screens for each sgRNA is shown. Each screen was normalized to the median effect of TGFBR1 sgRNAs and non-targeting control sgRNAs. Bar height represents the mean effect from a meta-analysis across all replicates using an inverse variance weighted analysis. Error bars depict standard error. The number of screens included for each sgRNA is shown under each respective sgRNA on the x-axis; within a single run of the assay each target sgRNA was included in 2-4 wells and each bar represents between 6 and 20 observations.

### DEFINITIONS

Unless defined otherwise, all technical and scientific terms used herein have the meaning commonly understood by a person skilled in the art to which this invention belongs. As used herein, the following terms have the meanings ascribed to them unless specified otherwise.

Cardiomyopathy is a group of diseases that affect the heart muscle. The term “cardiomyopathy” includes, but is not limited to, hypertrophic cardiomyopathy, dilated cardiomyopathy, restrictive cardiomyopathy, arrhythmogenic right ventricular dysplasia, and Takotsubo cardiomyopathy. At certain stages of the disease, cardiomyopathy patients may have no symptoms (e.g., in early stages of the disease). At other stages of the disease, symptoms of cardiomyopathy may include, but are not limited to, shortness of breath, fatigue, swelling of the legs, irregular heartbeat, and fainting. In some cases, cardiomyopathy may lead to heart failure. In certain cases, cardiomyopathy may result in sudden cardiac death. There is often a genetic basis for the development of cardiomyopathy, and in many cases, it is inherited within families. In other cases, causes of cardiomyopathy may include, but are not limited to, viral infections, coronary heart disease, drug use, alcohol use, or exposure to heavy metals. In some embodiments, cardiomyopathy is hypertrophic cardiomyopathy. Hypertrophic cardiomyopathy involves the thickening of the heart tissue and enlargement of the heart muscle, resulting in less efficient pumping of blood. In many cases, the causes of hypertrophic cardiomyopathy are unknown. In a majority of cases, hypertrophic cardiomyopathy is inherited within families and has a genetic basis. In some embodiments, the cardiomyopathy is dilated cardiomyopathy. Dilated cardiomyopathy involves the enlargement and weakening of the ventricles, resulting in inefficient pumping of blood. In many cases, the causes of dilated cardiomyopathy are unknown. In approximately a third of cases, dilated cardiomyopathy has a genetic basis and is inherited within families. Cardiomyopathy is further described in the following references, which are incorporated by reference herein: Hensley et al. Anesth. Analg. 2015, 120(3), 554-569; Schultheiss et al. 2019, Nat. Rev. Dis. Primers. 2019, 5(32); Braunwald et al. Circulation Res. 2017, 121(7), 711-721. The molecular mechanisms involved in cardiomyopathy are still poorly understood, limiting treatment options. Treatment of cardiomyopathy often focuses on managing the condition and includes lifestyle changes. Other treatment options vary depending on the type of cardiomyopathy but may include medication, the use of pacemakers (e.g., for slow heart rates), defibrillators (e.g., for patients prone to fatal heart rhythms), ventricular assist devices (e.g., in cases of severe heart failure), or ablation (e.g., for recurring dysrhythmias that cannot be eliminated by other means, such as medication). Medications for treatment of cardiomyopathy may include, without limitation, angiotensin-converting enzyme (ACE) inhibitors, angiotensin II receptor blockers, beta blockers, diuretics, digoxin, and blood-thinning medications. In some cases, treatment may ultimately involve a heart transplant.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or a genetically engineered animal. The term “patient” refers to a human subject in need of treatment of a disease. In some embodiments, the subject is human. In some embodiments, the patient is human. The human may be a male or female at any stage of development. A subject or patient “in need” of treatment of a disease or disorder (e.g., cardiomyopathy) includes, without limitation, those who exhibit any risk factors or symptoms of a disease or disorder. Such risk factors or symptoms may be, for example and without limitation, any of those associated with cardiomyopathy as discussed herein. The term “normal subject” as used herein refers to a healthy subject (e.g., a subject without cardiomyopathy).

The term “sample” or “biological sample” refers to any sample including tissue samples (such as tissue sections, surgical biopsies, and needle biopsies of a tissue); cell samples (e.g., cytological smears (such as Pap or blood smears) or samples of cells obtained by microdissection); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments, a biological sample is cardiac tissue (e.g., tissue from a heart biopsy). In some embodiments, a biological sample is a blood sample.

As used herein, the term “genetic signature” refers to a single or combined group of genes in a cell with a uniquely characteristic pattern of gene expression. A characteristic pattern of gene expression may occur as a result of a disease or disorder (e.g., cardiomyopathy). Gene signatures may be used for prognostic, diagnostic, and predictive applications (e.g., such signatures may be used to predict the survival or prognosis of an individual with a disease, or for differentiation between subtypes of a disease). Gene signatures may be observed in any cell type. In some embodiments, gene signatures are observed in fibroblasts (for example, activated fibroblasts). In some embodiments, gene signatures are observed in macrophages (for example, inflammatory macrophages). In certain embodiments, a gene signature comprises increased and/or decreased expression of one or more gene products or transcripts in a subject with a disease (e.g., cardiomyopathy), relative to a normal subject or a population of normal subjects. A genetic signature may be obtained or measured through any suitable method known in the art, including, but not limited to, RNA sequencing (e.g., single nuclei RNA sequencing) or antibody staining as discussed further herein. A genetic signature may comprise increased or decreased expression of a single gene product. In some embodiments, a genetic signature comprises increased and/or decreased expression of 2, 3, 4, 5, 6, 7, 8, 9, or 10 or more gene products.

The terms “treatment,” “treat,” and “treating” refer to reversing, alleviating, delaying the onset of, or inhibiting the progress of a disease described herein (e.g., cardiomyopathy). In some embodiments, treatment may be administered after one or more signs or symptoms of the disease have developed or have been observed (e.g., prophylactically (as may be further described herein) or upon suspicion or risk of disease). In other embodiments, treatment may be administered in the absence of signs or symptoms of the disease. For example, treatment may be administered to a susceptible subject prior to the onset of symptoms (e.g., in light of a history of symptoms in the subject, or family members of the subject). Treatment may also be continued after symptoms have resolved, for example, to delay or prevent recurrence. In some embodiments, treatment may be administered after observing a genetic signature associated with a particular disease (e.g., a genetic signature associated with cardiomyopathy. In certain embodiments, a treatment may be administered after observing a genetic signature associated with cardiomyopathy in combination with another test (e.g., a blood test, an echocardiogram, a physical exam, or a stress test).

The terms “administer,” “administering,” and “administration” refer to implanting, absorbing, ingesting, injecting, inhaling, or otherwise introducing an agent described herein (e.g., a small molecule, a nucleic acid, or a protein), or a composition thereof, in or on a subject.

A “therapeutically effective amount” of an agent described herein (e.g., a small molecule, a nucleic acid, or a protein) is an amount sufficient to provide a therapeutic benefit in the treatment of a condition (e.g., cardiomyopathy) or to delay or minimize one or more symptoms associated with the condition. A therapeutically effective amount of an agent means an amount of the agent, alone or in combination with other therapies, that provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for reducing expression of one or more gene products (e.g., gene products associated with activation of fibroblasts in cardiomyopathy). In some embodiments, a therapeutically effective amount is an amount sufficient for increasing expression of one or more gene products (e.g., gene products associated with activation of fibroblasts or reduction of proliferating macrophages in cardiomyopathy). In some embodiments, a therapeutically effective amount is an amount sufficient for inhibiting the activity of one or more gene products (e.g., gene products associated with activation of fibroblasts or reduction of proliferating macrophages in cardiomyopathy).

“Small molecules” include molecules, whether naturally occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The terms “polynucleotide,” “nucleotide sequence,” “nucleic acid,” “nucleic acid molecule,” “nucleic acid sequence,” and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore, a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region. In some embodiments, an siRNA may be used to target a gene product associated with cardiomyopathy and reduce expression of that gene product.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

An “antibody” refers to a glycoprotein belonging to the immunoglobulin superfamily. The terms antibody and immunoglobulin are used interchangeably. With some exceptions, mammalian antibodies are typically made of basic structural units each with two large heavy chains and two small light chain. There are several different types of antibody heavy chains, and several different kinds of antibodies, which are grouped together into different isotypes based on which heavy chain they possess. Five different antibody isotypes are known in mammals (IgG, IgA, IgE, IgD, and IgM), which perform different roles and help direct the appropriate immune response for each different type of foreign object they encounter. The term “antibody” as used herein also encompasses antibody fragments and nanobodies, as well as variants of antibodies and variants of antibody fragments and nanobodies. In some embodiments, an antibody binds a protein or enzyme encoded by any of the gene products associated with cardiomyopathy disclosed herein.

In some embodiments, “inhibit” or “inhibition” refers to a reduction (as compared to a baseline activity level, and as shall be understood when referring to a “reduction” herein) of the level of enzyme activity (e.g., the activity of any of the gene products associated with activation of cardiac fibroblasts disclosed herein) to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of activity (e.g., the level of activity in a normal subject or a population of normal subjects).

In certain embodiments, “increasing” the activity of a gene product refers to an increase (as compared to a baseline activity level, and as shall be understood when referring to an “increase” herein) of the level of enzyme activity, (e.g., the activity of any of the gene products associated with activation of cardiac fibroblasts disclosed herein) to a level that is 0.0001% more than, 0.001% more than, 0.01% more than, 0.1% more than, 0.5% more than, 1% more than, 2% more than, 3% more than, 4% more than, 5% more than, 10% more than, 20% more than, 30% more than, 40% more than, 50% more than, 75% more than, 100% more than, or more than 100% more than an initial level, which may, for example, be a baseline level of activity (e.g., the level of activity in a normal subject or a population of normal subjects).

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

The present disclosure provides methods for diagnosing cardiomyopathy in a subject. Also disclosed herein are methods for treating cardiomyopathy in a subject and methods for modulating the expression of gene products associated with the activation of cardiac fibroblasts. Further disclosed herein are compositions comprising an agent capable of modulating the expression of gene products associated with activation of cardiac fibroblasts. The present disclosure also provides kits comprising reagents for performing the methods disclosed herein.

### Methods of Diagnosing Cardiomyopathy

In one aspect, the disclosure relates to methods of diagnosing cardiomyopathy in a subject. In some embodiments, the methods comprise (a) providing a sample from a subject; and (b) evaluating the sample for the presence of a population of activated fibroblasts comprising a genetic signature. A genetic signature may comprise increased or decreased expression (e.g., relative to a sample provided from a normal subject or a population of normal subjects) of one or more gene products. In some embodiments, the one or more gene products are associated with the activation of cardiac fibroblasts. In some embodiments, the one or more gene products include periostin (POSTN), NADPH Oxidase 4 (NOX4), fibroblast activation protein (FAP), collagen type I alpha 1 chain (COL1A1), collagen type I alpha 2 chain (COL1A2), actin alpha 2 (ACTA2), solute carrier family 44 member 5 (SLC44A5), collagen type XXII alpha 1 chain (COL22A1), Ae binding protein 1 (AEBP1), thrombospondin-4 (THBS4), family with sequence similarity 155, member A (FAM155A), teashirt zinc finger homeobox 2 (TSHZ2), juxtaposed with another zinc finger protein 1 (JAZF1), proline and arginine rich end leucine rich repeat protein (PRELP), calsyntenin 2 (CLSTN2), integrin alpha 10 (ITGA10), cell adhesion molecule 1 (CADM1), neuronal growth regulator 1 (NEGR1), platelet-derived growth factor receptor A (PDGFRA), complement component 7 (C7), fibulin 5 (FBLN5), and/or collagen type IV alpha 4 chain (COL4A4). In some embodiments, the one or more gene products include periostin (POSTN), NADPH Oxidase 4 (NOX4), fibroblast activation protein (FAP), collagen type I alpha 1 chain (COL1A1), collagen type I alpha 2 chain (COL1A2), actin alpha 2 (ACTA2), solute carrier family 44 member 5 (SLC44A5), collagen type XXII alpha 1 chain (COL22A1), Ae binding protein 1 (AEBP1), thrombospondin-4 (THBS4), family with sequence similarity 155, member A (FAM155A), teashirt zinc finger homeobox 2 (TSHZ2), neuronal growth regulator 1 (NEGR1), platelet-derived growth factor receptor A (PDGFRA), complement component 7 (C7), fibulin 5 (FBLN5), and/or collagen type IV alpha 4 chain (COL4A4). In certain embodiments, the one or more gene products include POSTN. In certain embodiments, the one or more gene products include COL22A1. In certain embodiments, the one or more gene products include THBS4. In certain embodiments, the one or more gene products include JAZF1. In certain embodiments, the genetic signature comprises increased or decreased expression of one or more genes selected from the group consisting of NEGR1, FBLN5, PRELP, CLSTN2, ITGA10, JAZF1, COL22A1, AEBP1, FGF14, THBS4, FAP, and CADM1.

The subject being diagnosed may be suspected of having cardiomyopathy prior to undergoing the methods for diagnosis described herein. For example, the subject may have one or more of the symptoms of cardiomyopathy disclosed herein (e.g., shortness of breath, fatigue, swelling of the legs, irregular heartbeat, and fainting). The subject being diagnosed may also be at risk of having cardiomyopathy. For example, the subject may have a family history of cardiomyopathy, as discussed herein. In some embodiments, the subject being diagnosed is not suspected of having or thought to be at risk for having cardiomyopathy.

Diagnosis of any type of cardiomyopathy (e.g., hypertrophic cardiomyopathy, dilated cardiomyopathy, restrictive cardiomyopathy, arrhythmogenic right ventricular dysplasia, or Takotsubo cardiomyopathy) is contemplated by the present disclosure. In some embodiments, the cardiomyopathy is dilated cardiomyopathy. In some embodiments, the cardiomyopathy is hypertrophic cardiomyopathy.

The step of evaluating a sample for increased or decreased expression of gene products associated with activated cardiac fibroblasts in cardiomyopathy may be accomplished by any methods known in the art. In some embodiments, the step of evaluating a sample comprises performing RNA sequencing on the sample using various methods known in the art (e.g., as disclosed in Stark et al., Nature Reviews Genetics 2019, 20, 631-656; Chen et al. Front. Genet. 2019, 10(317); and Costa-Silva et al. PLOS ONE 2017, 12(12), e0190152). RNA sequencing may include single-cell RNA sequencing or single-nuclei RNA sequencing. In some embodiments, the step of evaluating a sample comprises staining the sample with an antibody for a gene product of interest. The antibody may then be detected and quantified using various methods known in the art (e.g., using fluorescence assays, immunoprecipitation assays, immunoblotting, or immunosorbent assays).

The methods disclosed herein contemplate evaluation of any sample provided from a subject. In some embodiments, the sample is a tissue sample (e.g., the sample is obtained from a myocardial biopsy). In some embodiments, the sample is a blood sample provided from a subject.

A genetic signature evaluated by the methods disclosed herein comprises genes and gene products that are differentially expressed (e.g., relative to a sample provided from a normal subject or a population of normal subjects). In some embodiments, a genetic signature comprises increased expression, relative to a sample provided from a normal subject or a population of normal subjects, of one or more gene products. Gene products that have been found to be expressed at a higher level in subjects with cardiomyopathy may include, but are not limited to, POSTN, NOX4, FAP, COL1A1, COL1A2, ACTA2, SLC44A5, COL22A1, AEBP1, THBS4, FAM155A, JAZF1, PRELP, CLSTN2, ITGA10, CADM1, and TSHZ2. In some embodiments, gene products that have been found to be expressed at a higher level in subjects with cardiomyopathy may include, but are not limited to, POSTN, NOX4, FAP, COL1A1, COL1A2, ACTA2, SLC44A5, COL22A1, AEBP1, THBS4, FAM155A, and TSHZ2. In certain embodiments, a genetic signature comprises decreased expression, relative to a sample provided from a normal subject or a population of normal subjects, of one or more gene products. Gene products that have been found to be expressed at lower levels in subjects with cardiomyopathy may include, but are not limited to, NEGR1, PDGFRA, C7, FBLN5, and COL4A4. In certain embodiments, the genetic signature comprises increased or decreased expression of one or more genes selected from the group consisting of NEGR1, FBLN5, PRELP, CLSTN2, ITGA10, JAZF1, COL22A1, AEBP1, FGF14, THBS4, FAP, and CADM1.

A genetic signature may comprise increased or decreased expression, relative to a sample provided from a normal subject or a population of normal subjects, of one or more gene products associated with activation of cardiac fibroblasts. In some embodiments, a genetic signature comprises increased or decreased expression of two or more gene products. In some embodiments, a genetic signature comprises increased expression of three or more gene products. In certain embodiments, a genetic signature comprises increased expression of four or more, five or more, six or more, seven or more, eight or more, nine or more, or ten or more gene products.

A genetic signature may consist of differential expression of COL22A1 (e.g., increased expression of COL22A1 relative to a sample provided from a normal subject or a population of normal subjects). A genetic signature may also consist of differential expression of POSTN (e.g., increased expression of POSTN relative to a sample provided from a normal subject or a population of normal subjects). A genetic signature may also consist of differential expression of THBS4 (e.g., increased expression of THBS4 relative to a sample provided from a normal subject or a population of normal subjects). In some embodiments, a genetic signature includes two of the three gene products in the group consisting of COL22A1, POSTN, and THBS4, and optionally one or more of the other gene products associated with cardiomyopathy provided herein. In some embodiments, a genetic signature includes COL22A1, POSTN, and THBS4, and optionally one or more of the other gene products associated with cardiomyopathy provided herein.

In some embodiments, a genetic signature comprises increased or decreased expression, relative to a sample provided from a normal subject or a population of normal subjects, of one or more of the gene products associated with cardiomyopathy provided herein, wherein one of the gene products is COL22A1. In certain embodiments, a genetic signature comprises increased or decreased expression, relative to a sample provided from a normal subject or a population of normal subjects, of two or more of the gene products associated with cardiomyopathy provided herein, wherein one of the gene products is COL22A1. In certain embodiments, a genetic signature comprises increased or decreased expression, relative to a sample provided from a normal subject or a population of normal subjects, of three or more of the gene products associated with cardiomyopathy provided herein, wherein one of the gene products is COL22A1.

In some embodiments, a genetic signature comprises increased or decreased expression, relative to a sample provided from a normal subject or a population of normal subjects, of one or more of the gene products associated with cardiomyopathy provided herein, wherein one of the gene products is POSTN. In certain embodiments, a genetic signature comprises increased or decreased expression, relative to a sample provided from a normal subject or a population of normal subjects, of two or more of the gene products associated with cardiomyopathy provided herein, wherein one of the gene products is POSTN. In certain embodiments, a genetic signature comprises increased or decreased expression, relative to a sample provided from a normal subject or a population of normal subjects, of three or more of the gene products associated with cardiomyopathy provided herein, wherein one of the gene products is POSTN.

In some embodiments, a genetic signature comprises increased or decreased expression, relative to a sample provided from a normal subject or a population of normal subjects, of one or more of the gene products associated with cardiomyopathy provided herein, wherein one of the gene products is THBS4. In certain embodiments, a genetic signature comprises increased or decreased expression, relative to a sample provided from a normal subject or a population of normal subjects, of two or more gene products, wherein one of the of the gene products associated with cardiomyopathy provided herein is THBS4. In certain embodiments, a genetic signature comprises increased or decreased expression, relative to a sample provided from a normal subject or a population of normal subjects, of three or more of the gene products associated with cardiomyopathy provided herein, wherein one of the gene products is THBS4.

### Methods of Treating Cardiomyopathy

In another aspect, this disclosure relates to methods of treating cardiomyopathy in a subject in need thereof. In some embodiments, the methods of treatment comprise administering a treatment to the subject based on the presence of a population of activated fibroblasts comprising a genetic signature, as described herein. The use of any treatment for cardiomyopathy known in the art is contemplated, including, but not limited to, lifestyle changes (e.g., exercise), use of a pacemaker, use of a defibrillator, use of a ventricular assist device, ablation, an angiotensin-converting enzyme (ACE) inhibitor, an angiotensin II receptor blocker, a beta blocker, a diuretic, digoxin, a blood-thinning medication, or a heart transplant. In some embodiments, the methods comprise administering to a subject a therapeutically effective amount of an agent capable of modulating the activity of one or more gene products associated with activation of cardiac fibroblasts. Such gene products may include any of those disclosed herein (e.g., POSTN, NOX4, FAP, COL1A1, COL1A2, ACTA2, SLC44A5, COL22A1, AEBP1, THBS4, FAM155A, TSHZ2, JAZF1, PRELP, CLSTN2, ITGA10, CADM1, NEGR1, PDGFRA, C7, FBLN5, and COL4A4). In some embodiments, such gene products include POSTN, NOX4, FAP, COL1A1, COL1A2, ACTA2, SLC44A5, COL22A1, AEBP1, THBS4, FAM155A, TSHZ2, NEGR1, PDGFRA, C7, FBLN5, and COL4A4.

Subjects to whom treatment is contemplated by the present disclosure may be suspected of having cardiomyopathy (e.g., the subject may have one or more of the symptoms of cardiomyopathy disclosed herein, including shortness of breath, fatigue, swelling of the legs, an irregular heartbeat, and/or fainting). A subject to whom treatment is contemplated may also be at risk of having cardiomyopathy. For example, the subject may have a family history of cardiomyopathy, as discussed herein. In some embodiments, a subject may have a disease or condition that may lead to cardiomyopathy. In some embodiments, a subject may have been diagnosed with cardiomyopathy prior to being treated using the methods disclosed herein. In certain embodiments, a subject is diagnosed with cardiomyopathy using the methods disclosed herein, and then treated for cardiomyopathy (e.g., dilated cardiomyopathy or hypertrophic cardiomyopathy) using the methods disclosed herein or other methods and therapies known in the art as described herein, or any other methods for diagnosis known in the art.

Treatment of subjects with various agents is contemplated by the present disclosure. Such agents may include, but are not limited to, small molecules, nucleic acids, and proteins. In some embodiments, the agent is a small molecule. For example, the agent may be a small molecule capable of inhibiting the activity of one of the gene products associated with activation of cardiac fibroblasts disclosed herein. The agent may also be a small molecule capable of inhibiting the expression or inducing the expression of one or more of the gene products associated with activation of cardiac fibroblasts disclosed herein. In some embodiments, the agent is a nucleic acid. In some embodiments, the nucleic acid is an mRNA, an antisense RNA, an miRNA, an siRNA, an RNA aptamer, a double stranded RNA (dsRNA), a short hairpin RNA (shRNA), or an antisense oligonucleotide (ASO). For example, the agent may be an siRNA capable of inhibiting or reducing the expression of one of the gene products associated with activation of cardiac fibroblasts disclosed herein. In some embodiments, the agent is a protein. In certain embodiments, the agent is an antibody (e.g., an antibody directed to a protein encoded by any of the gene products associated with cardiomyopathy disclosed herein). In some embodiments, administering the agent prevents activation of cardiac fibroblasts. In certain embodiments, administering the agent induces activation of cardiac fibroblasts. An agent may also be a known treatment for cardiomyopathy (e.g., an angiotensin-converting enzyme (ACE) inhibitor, an angiotensin II receptor blocker, a beta blocker, a diuretic, digoxin, or a blood-thinning medication). Such agents may be used to treat a subject based on the presence of a specific genetic signature, as disclosed herein. For example, ACE inhibitors include, but are not limited to, sulfhydryl-containing agents (e.g., alacepril, captopril, and zofenopril), dicarboxylate-containing agents (e.g., enalapril, ramipril, quinapril, perindopril, lisinopril, benazepril, imidapril, trandolapril, and cilazapril), and phosphonate-containing agents (e.g., fosinopril). Angiotensin II receptor blockers include, but are not limited to, olmesartan, valsartan, losartan, telmisartan, azilsartan medoxomil, irbesartan, candesartan, eprosartan, and valsartan. Beta blockers include, but are not limited to, propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, ICI-118,551, SR 59230A, and nebivolol. Diuretics include, but are not limited to, high ceiling/loop diuretics, thiazides such as hydrochlorothiazide, carbonic anhydrase inhibitors, potassium-sparing diuretics, calcium-sparing diuretics, osmotic diuretics, and low ceiling diuretics. Blood-thinning medications include, but are not limited to, dabigatran, apixaban, rivaroxaban, warfarin, aspirin, clopidogrel, prasugrel, and ticagrelor.

The various agents described herein are capable of modulating the activity of one or more gene products. Modulating the activity of one or more gene products may comprise decreasing the expression of the gene products. Modulating the activity of one or more gene products may also comprise inhibiting the activity of the gene products (e.g., with small molecule inhibitors). In some embodiments, the methods disclosed herein contemplate decreasing the expression of or inhibiting the activity of one or more of the gene products selected from the group consisting of POSTN, NOX4, FAP, COL1A1, COL1A2, ACTA2, SLC44A5, COL22A1, AEBP1, THBS4, FAM155A, JAZF1, PRELP, CLSTN2, ITGA10, CADM1, and TSHZ2. In some embodiments, the methods disclosed herein contemplate decreasing the expression of or inhibiting the activity of one or more of the gene products selected from the group consisting of POSTN, NOX4, FAP, COL1A1, COL1A2, ACTA2, SLC44A5, COL22A1, AEBP1, THBS4, FAM155A, and TSHZ2. Modulating the activity of one or more gene products may also comprise increasing the expression of the gene products. In some embodiments, the methods disclosed herein contemplate increasing the expression of one or more of the gene products NEGR1, PDGFRA, C7, FBLN5, and COL4A4. In certain embodiments, the genetic signature comprises increased or decreased expression of one or more genes selected from the group consisting of NEGR1, FBLN5, PRELP, CLSTN2, ITGA10, JAZF1, COL22A1, AEBP1, FGF14, THBS4, FAP, and CADM1.

The methods of treatment disclosed herein encompass administering one or more agents to increase or decrease the activity of one or more gene products associated with activation of cardiac fibroblasts. In some embodiments, such methods comprise administering one or more agents to increase or decrease the activity of two or more gene products. In some embodiments, such methods comprise administering one or more agents to increase or decrease the activity of three or more gene products.

In some embodiments, an agent is administered to modulate the activity of COL22A1 (e.g., decrease expression of COL22A1 or inhibit the activity of COL22A1 relative to a sample provided from a normal subject or a population of normal subjects). An agent may also be administered to modulate the activity of POSTN (e.g., decrease expression of POSTN or inhibit the activity of POSTN relative to a sample provided from a normal subject or a population of normal subjects). In certain embodiments, an agent is administered to modulate expression of THBS4 (e.g., decrease expression of THBS4 or inhibit the activity of THBS4 relative to a sample provided from a normal subject or a population of normal subjects).

In some embodiments, an agent capable of modulating the activity of one or more of the gene products associated with cardiomyopathy provided herein is administered, wherein one of the gene products is COL22A1. In certain embodiments, an agent capable of modulating the activity of two or more of the gene products associated with cardiomyopathy provided herein is administered, wherein one of the gene products is COL22A1. In certain embodiments, an agent capable of modulating the activity of three or more of the gene products associated with cardiomyopathy provided herein is administered, wherein one of the gene products is COL22A1.

In some embodiments, an agent capable of modulating the activity of one or more of the gene products associated with cardiomyopathy provided herein is administered, wherein one of the gene products is POSTN. In certain embodiments, an agent capable of modulating the activity of two or more of the gene products associated with cardiomyopathy provided herein is administered, wherein one of the gene products is POSTN. In certain embodiments, an agent capable of modulating the activity of three or more of the gene products associated with cardiomyopathy provided herein is administered, wherein one of the gene products is POSTN.

In some embodiments, an agent capable of modulating the activity of one or more of the gene products associated with cardiomyopathy provided herein is administered, wherein one of the gene products is THBS4. In certain embodiments, an agent capable of modulating the activity of two or more of the gene products associated with cardiomyopathy provided herein is administered, wherein one of the gene products is THBS4. In certain embodiments, an agent capable of modulating the activity of three or more of the gene products associated with cardiomyopathy provided herein is administered, wherein one of the gene products is THBS4.

Methods of Modulating Expression of Gene Products Associated with Activated Cardiac Fibroblasts

In another aspect, this disclosure relates to methods of modulating expression of one or more gene products associated with activated cardiac fibroblasts in a subject in need thereof. In some embodiments, the method comprises administering to the subject any of the agents as described herein, which are capable of modulating the activity of one or more gene products associated with the activation of cardiac fibroblasts. The one or more gene products may be selected from the group consisting of POSTN, NOX4, FAP, COL1A1, COL1A2, ACTA2, SLC44A5, COL22A1, AEBP1, THBS4, FAM155A, JAZF1, PRELP, CLSTN2, ITGA10, CADM1, and TSHZ2. In some embodiments, the one or more gene products are selected from the group consisting of POSTN, NOX4, FAP, COL1A1, COL1A2, ACTA2, SLC44A5, COL22A1, AEBP1, THBS4, FAM155A, and TSHZ2. Modulating the expression of such gene products may be accomplished using any of the methods disclosed herein or any method known in the art, in any of the subjects disclosed herein (e.g., subjects who have been diagnosed with cardiomyopathy using the methods described herein).

In some embodiments, an agent is administered to modulate the activity of COL22A1 (e.g., decrease expression of COL22A1 or inhibit the activity of COL22A1 relative to a sample provided from a normal subject or a population of normal subjects). An agent may also be administered to modulate the activity of POSTN (e.g., decrease expression of POSTN or inhibit the activity of POSTN relative to a sample provided from a normal subject or a population of normal subjects). In certain embodiments, an agent is administered to modulate expression of THBS4 (e.g., decrease expression of THBS4 or inhibit the activity of THBS4 relative to a sample provided from a normal subject or a population of normal subjects). In certain embodiments, the agent is administered to modulate the activity or expression of one or more genes selected from the group consisting of NEGR1, FBLN5, PRELP, CLSTN2, ITGA10, JAZF1, COL22A1, AEBP1, FGF14, THBS4, FAP, and CADM1.

### Compositions

The present disclosure also provides compositions comprising any of the agents described herein (e.g., a small molecule, nucleic acid, or protein capable of modulating the activity of one or more gene products associated with activation of cardiac fibroblasts) in an effective amount. In some embodiments, combinations of one or more agents are provided in a composition in an effective amount. In certain embodiments, the effective amount is a therapeutically effective amount. In certain embodiments, the effective amount is an amount effective for treating cardiomyopathy (e.g., dilated cardiomyopathy or hypertrophic cardiomyopathy) in a subject in need thereof. In certain embodiments, the effective amount is an amount effective for preventing cardiomyopathy (e.g., dilated cardiomyopathy or hypertrophic cardiomyopathy) in a subject in need thereof. In certain embodiments, the effective amount is an amount effective for reducing the risk of developing cardiomyopathy (e.g., dilated cardiomyopathy or hypertrophic cardiomyopathy) in a subject in need thereof. In certain embodiments, the effective amount is an amount effective for inhibiting the activity of any of the gene products associated with activation of cardiac fibroblasts disclosed herein in a subject or cell. In certain embodiments, the composition contains a second agent. In some embodiments, the second agent is a small molecule. In some embodiments, the second agent is a nucleic acid (e.g., an mRNA, an antisense RNA, an miRNA, an siRNA, an RNA aptamer, a double stranded RNA (dsRNA), a short hairpin RNA (shRNA), or an antisense oligonucleotide (ASO)). In certain embodiments, the second agent is an siRNA. In some embodiments, the second agent is a protein. In certain embodiments, the second agent is an antibody.

Compositions described herein can be prepared by any method known in the art of pharmacology. In general, such preparatory methods include bringing the agent(s) described herein (i.e., the “active ingredient(s)”) into association with a carrier or excipient, and/or one or more other accessory ingredients, and then, if necessary and/or desirable, shaping, and/or packaging the product into a desired single- or multi-dose unit. Compositions can be prepared, packaged, and/or sold in bulk, as a single unit dose, and/or as a plurality of single unit doses. A “unit dose” is a discrete amount of the composition comprising a predetermined amount of the active ingredient. The amount of the active ingredient is generally equal to the dosage of the active ingredient which would be administered to a subject and/or a convenient fraction of such a dosage, such as one-half or one-third of such a dosage. Relative amounts of the active ingredient(s), the pharmaceutically acceptable excipient, and/or any additional ingredients in a pharmaceutical composition described herein will vary, depending upon the identity, size, and/or condition of the subject treated, and further depending upon the route by which the composition is to be administered. The composition may comprise between 0.1% and 100% (w/w) active ingredient(s).

Pharmaceutically acceptable excipients used in the manufacture of provided compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors, including the nature of the agent (e.g., its stability in the environment of the gastrointestinal tract), and/or the condition of the subject (e.g., whether the subject is able to tolerate oral administration).

### Kits

Also encompassed by the disclosure are kits (e.g., pharmaceutical packs). The kits provided may comprise a composition or agent described herein and a container (e.g., a vial, ampule, bottle, syringe, and/or dispenser package, or other suitable container). In some embodiments, the provided kits may optionally further include a second container comprising a pharmaceutical excipient for dilution or suspension of a composition or agent described herein. In some embodiments, the composition or agent described herein provided in the first container and the second container are combined to form one unit dosage form.

Thus, in one aspect, provided are kits including a first container comprising agent(s) or composition(s) described herein. In certain embodiments, the kits are useful for treating a disease (e.g., cardiomyopathy) in a subject in need thereof. In certain embodiments, the kits are useful for preventing a disease (e.g., cardiomyopathy) in a subject in need thereof. In certain embodiments, the kits are useful for reducing the risk of developing a disease (e.g., cardiomyopathy) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. A kit described herein may include one or more additional pharmaceutical agents described herein as a separate composition.

In one aspect, the disclosure relates to kits for treating or for diagnosing subjects having, at risk of having, or suspected of having cardiomyopathy. In some embodiments, a kit for diagnosing such subjects comprises reagents for performing any of the methods described herein. Reagents for performing such methods may include, for example, reagents for performing RNA sequencing (e.g., primers, gene chips, DNase, polymerases) or reagents for antibody staining.

### EXAMPLES

### Example 1: Cellular Composition of Left Ventricle Samples from Dilated Cardiomyopathy (DCM) Patients, Hypertrophic Cardiomyopathy (HCM) Patients, and Healthy (NF) Patients

Given the immense public health burden of heart failure (HF), understanding the underlying mechanisms at a molecular level has been at the forefront of cardiovascular research. One common cause of HF, dilated cardiomyopathy (DCM), manifests as dilation of the left ventricle (LV) with systolic dysfunction, but normal LV thickness.⁵ Conversely, hypertrophic cardiomyopathy (HCM) is a heterogeneous disease characterized by a thickening of the LV wall and is often due to genetic mutations in sarcomere genes.⁵ Considering the complex etiology of HF, molecular phenotyping combined with careful examination of clinical phenotypes may yield unique insights into disease progression. Amongst these is the regulation of gene expression, where tissue level comparisons of RNA and protein have uncovered disease-specific transcriptional programs.^{3,4,6} With recent advances in single cell and single nucleus RNA sequencing technologies, it was sought to understand these transcriptional changes at single cell resolution.⁷⁻⁹

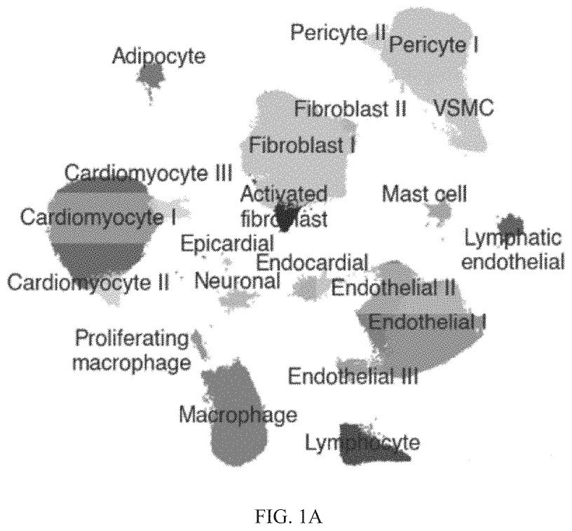

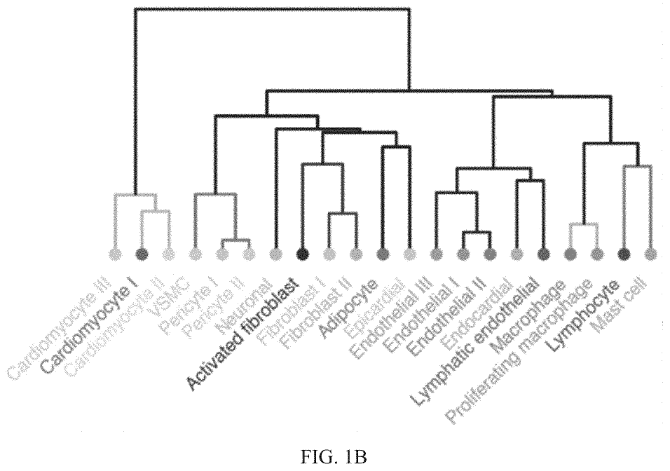

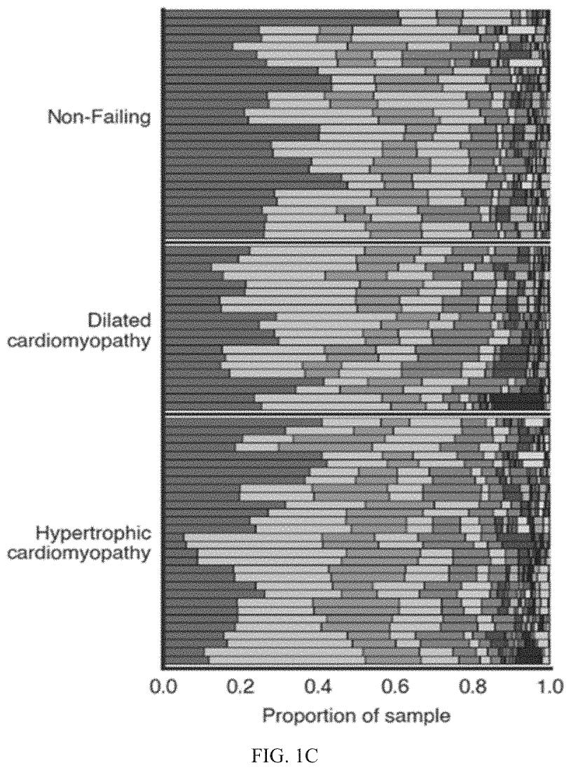

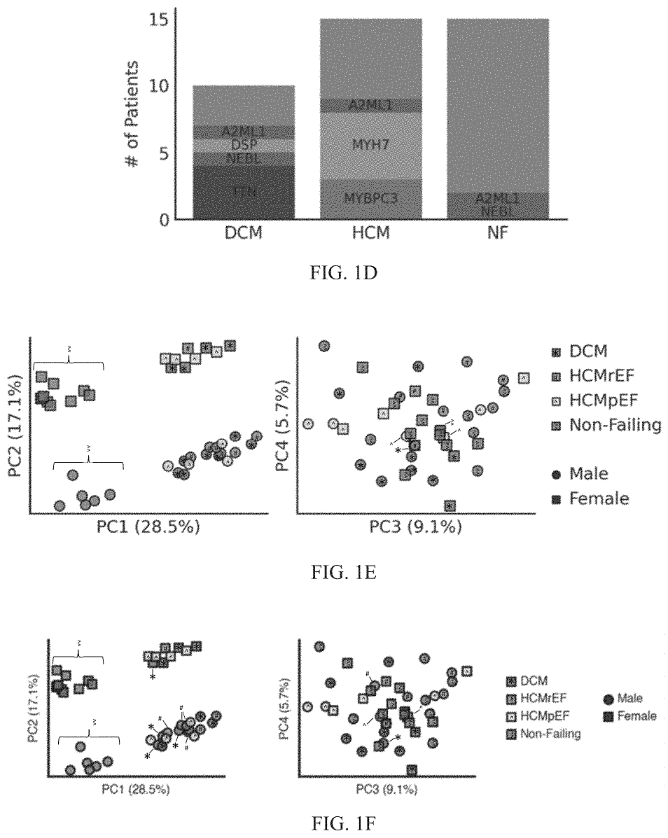

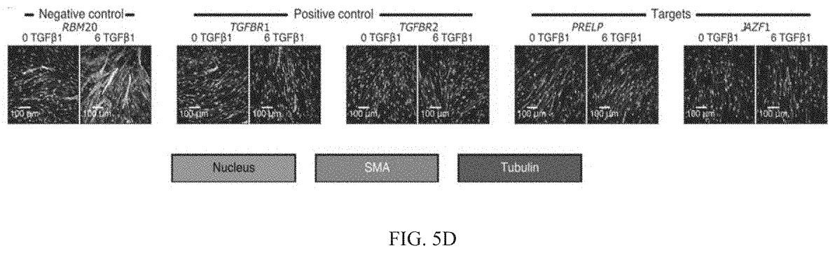

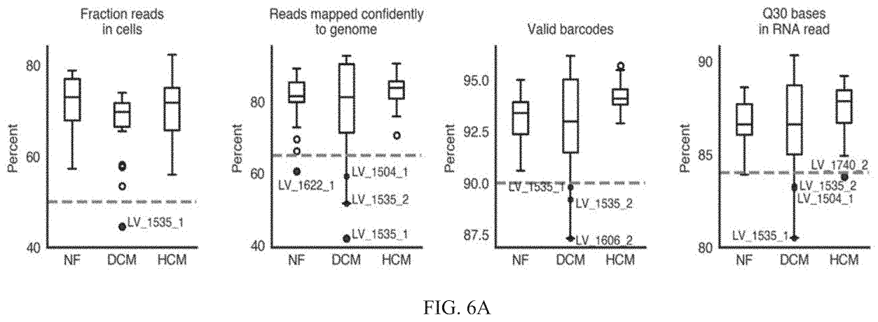

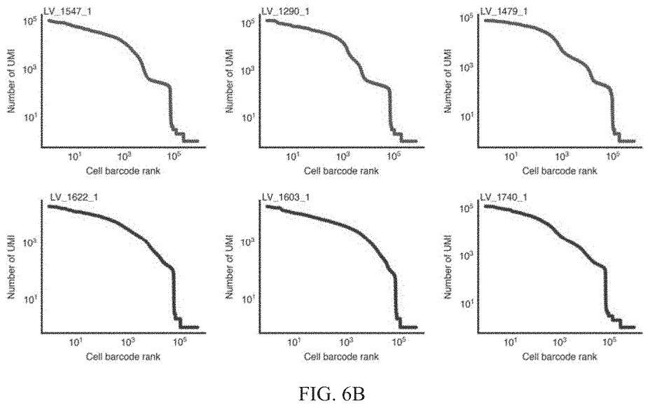

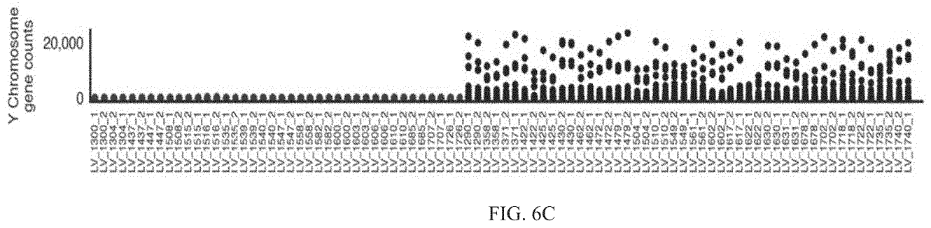

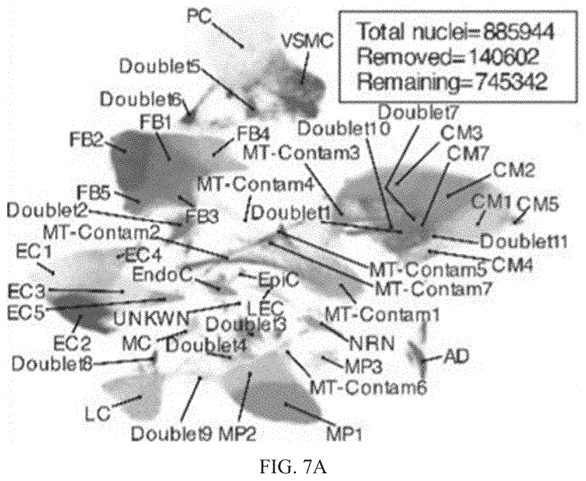

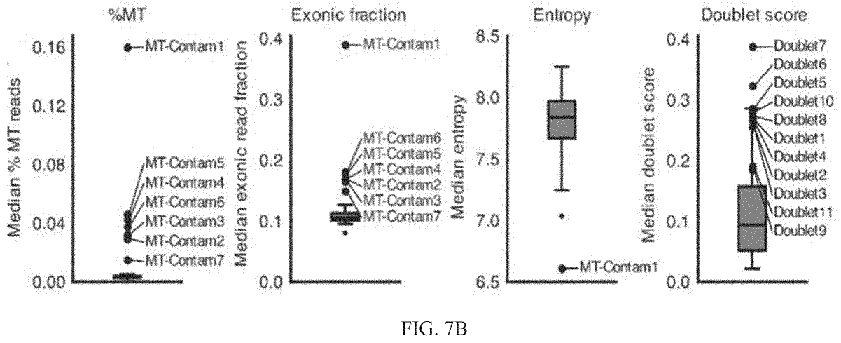

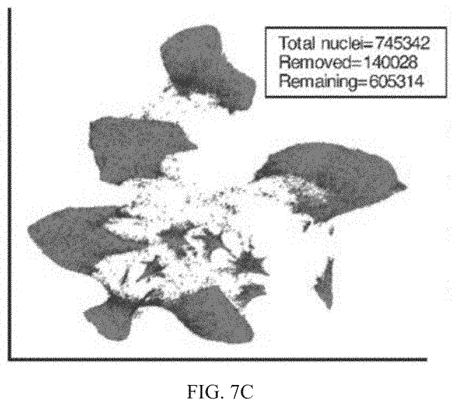

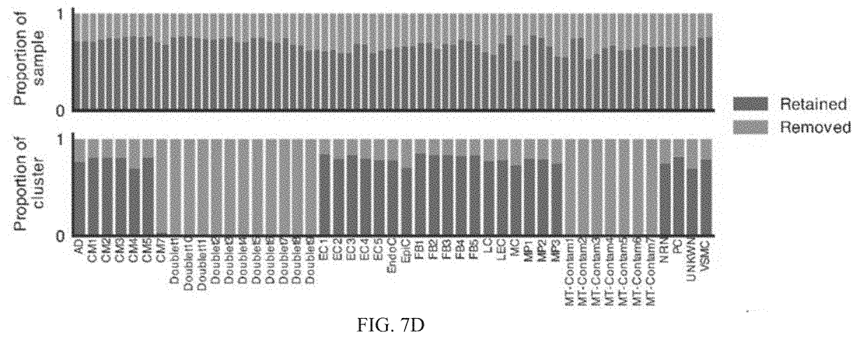

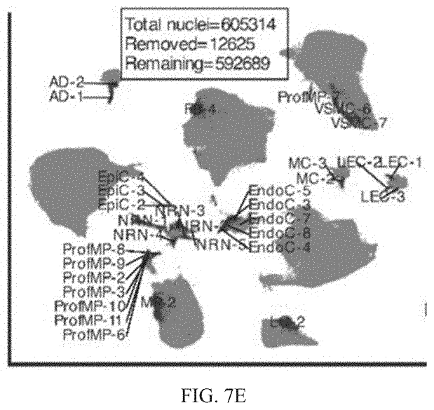

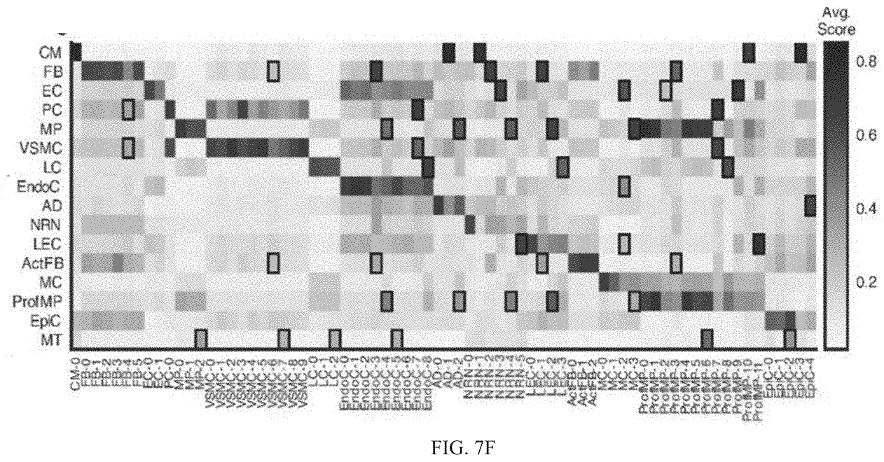

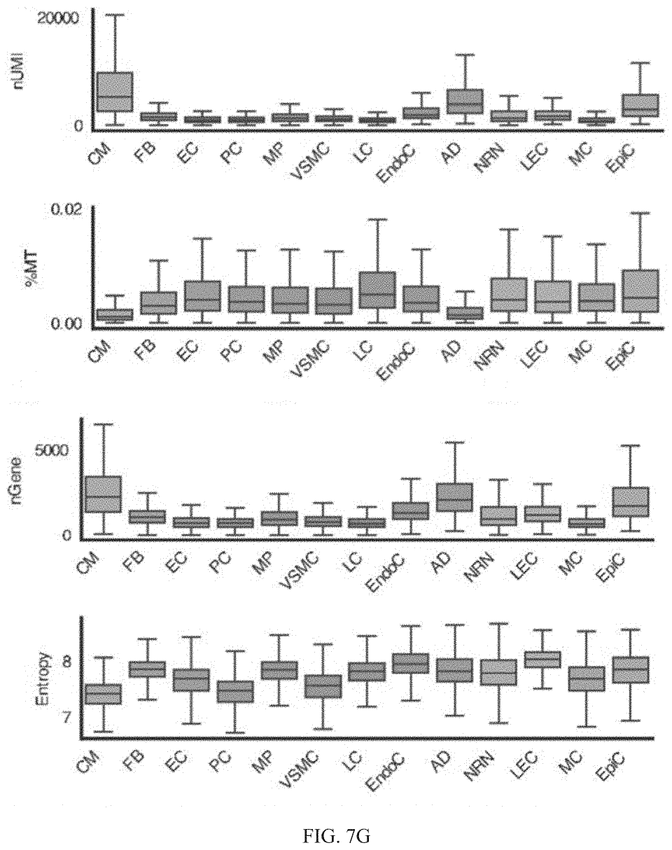

To better understand the transcriptional profile of DCM, HCM and healthy patients, snRNA-seq was performed in replicate on left ventricle samples from 42 patients including 12 with DCM, 16 with HCM, and 16 non-failing (NF) hearts. After strict quality control, 8 samples were removed, leaving at least one technical replicate from 11 DCM, 15 HCM, and 16 NF left ventricles (FIGS. 6A-6C). All DCM and HCM patients had advanced cardiomyopathy requiring transplantation. Left ventricular ejection fraction (LVEF) was under 20% for DCM patients, whereas seven HCM patients had reduced ejection fraction (LVEF<50%) and 8 HCM patients had preserved ejection fraction (LVEF≥50%). After removal of low-quality nuclei (FIGS. 7A-7G), a total of 592,689 nuclei were aggregated into 21 clusters based on transcriptional similarity (FIG. 1A; FIG. 1B). Genes selectively expressed in each cluster were identified and investigated for enrichment in biological processes based on gene ontology¹⁰⁻¹¹ for these gene sets (FIGS. 7A-7G). Based on these marker genes and biological processes, cell type labels were assigned to each cluster. Consistent with previous work⁷, the most abundant cell types were cardiomyocytes, fibroblasts, endothelial cells, mural cells, macrophages, and lymphocytes (FIG. 1C). The distribution of cell types across experiments was relatively uniform apart from an appreciably sized activated fibroblast population largely derived from DCM patient P1304 and HCM patient P1425 (FIG. 1C). Compared to NF hearts, both DCM and HCM hearts had a statistically credible decrease in cardiomyocytes and increases in vascular smooth muscle cells (VSMCs) and activated fibroblasts. Additionally, DCM hearts had statistically credible increases in fibroblasts and macrophages compared to NF hearts, whereas HCM hearts had an increase in lymphatic endothelial cells compared to NF hearts.

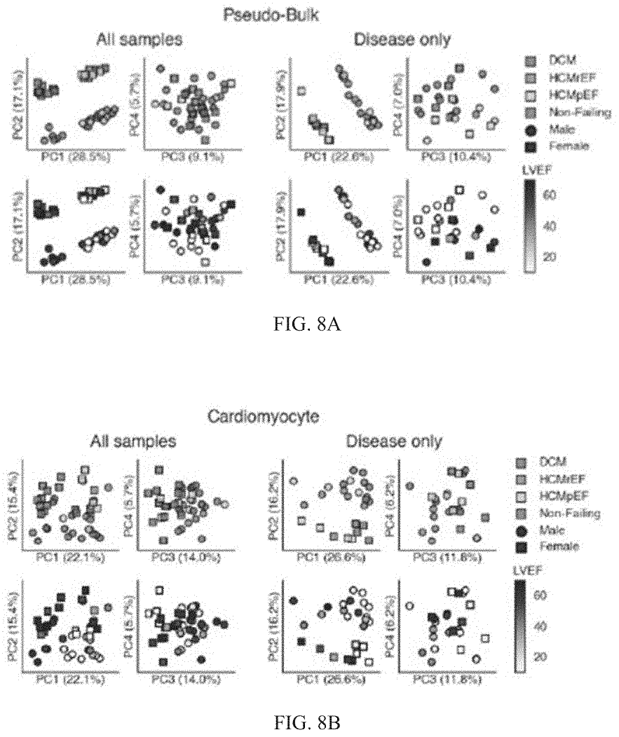

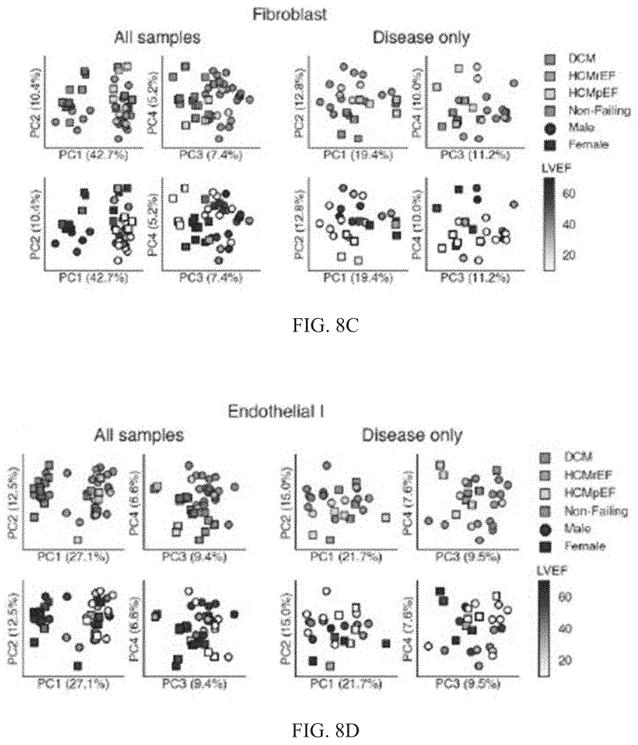

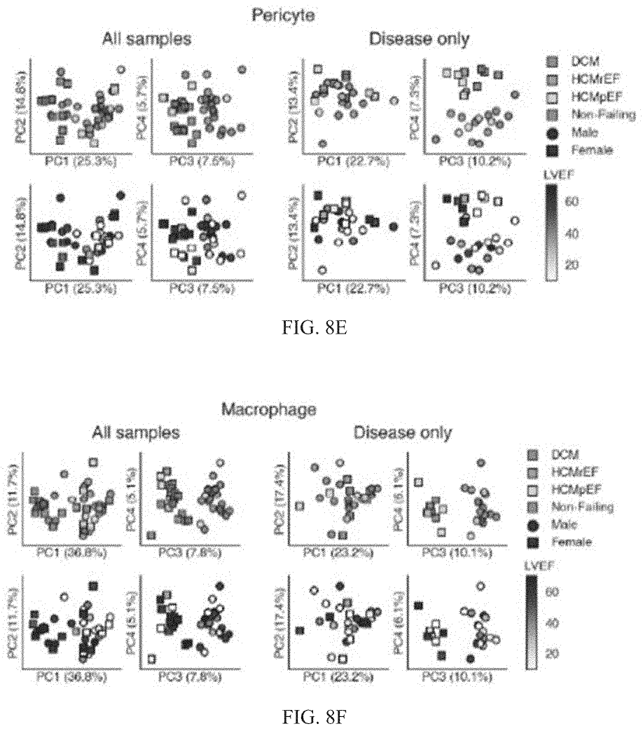

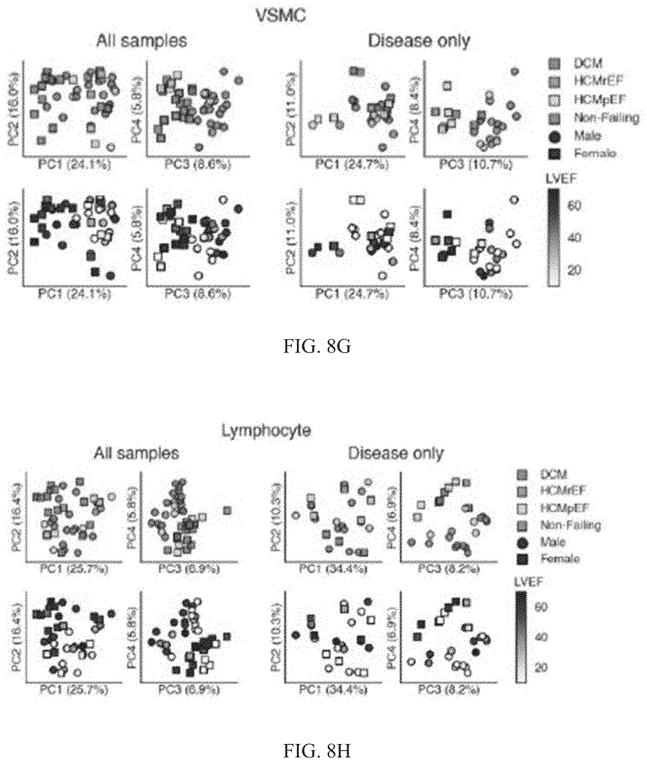

To further classify DCM and HCM patients based on known genetic drivers of disease, whole-genome sequencing was performed on the patients included in this study. The expected enrichments of TTN loss-of-function (LOF) carriers in DCM (4/10), MYBPC3 LOF carriers in HCM (3/15), and MYH7 pathogenic variant carriers in HCM (5/15) were observed (FIG. 1D). Based on principal components analysis (PCA) across the combination of all cell types per sample (“pseudo-bulk”), the strongest source of transcriptional variation separated DCM and HCM patients from NF patients (FIG. 1E). The second largest source of variation divided patients by sex. This pattern was generally consistent when clustering patients within a given cell type (FIGS. 8A-8H). When performing PCA restricted to DCM and HCM patients using pseudo-bulk data, modest separation of all patients was observed based on LVEF across principal component 4, which explains ˜7% of variation (FIGS. 8A-8H).

### Example 2: Identification of Differentially Expressed Genes Between Cardiomyopathy and Healthy Patient Heart Samples

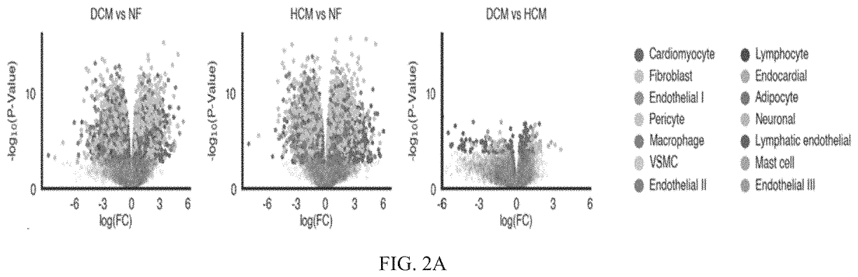

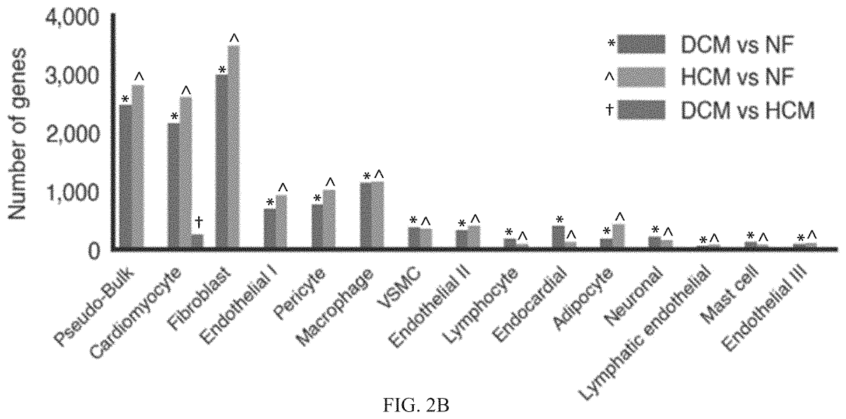

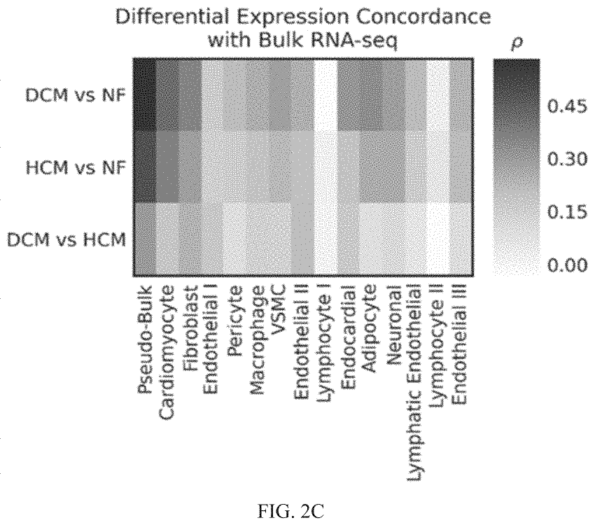

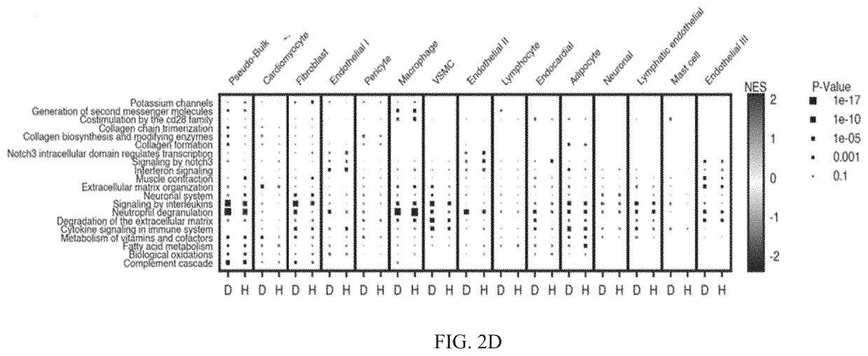

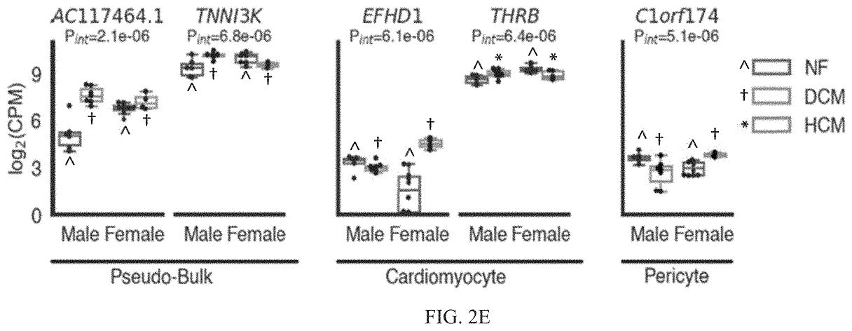

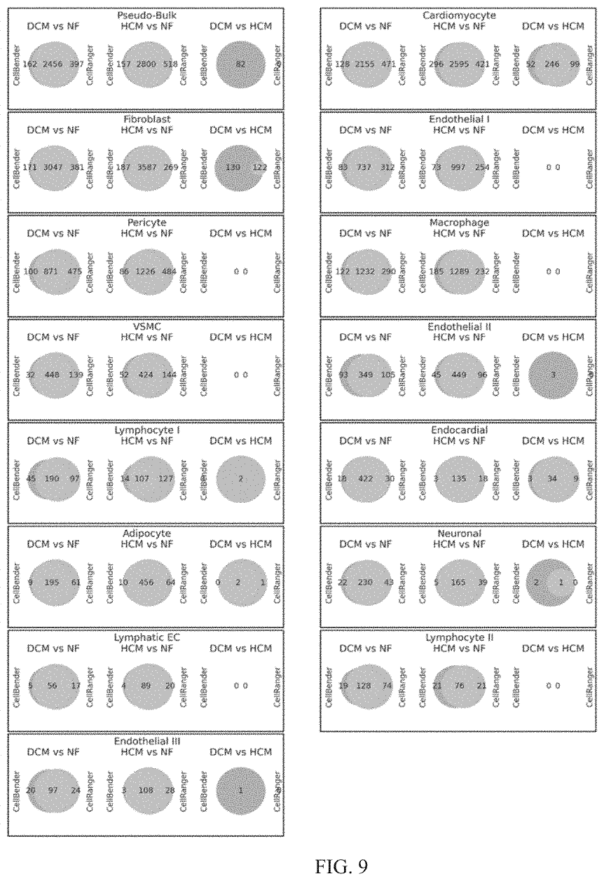

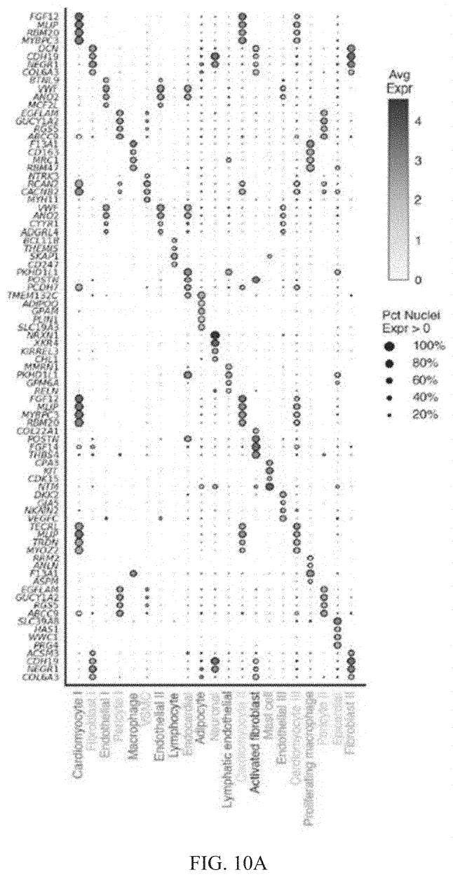

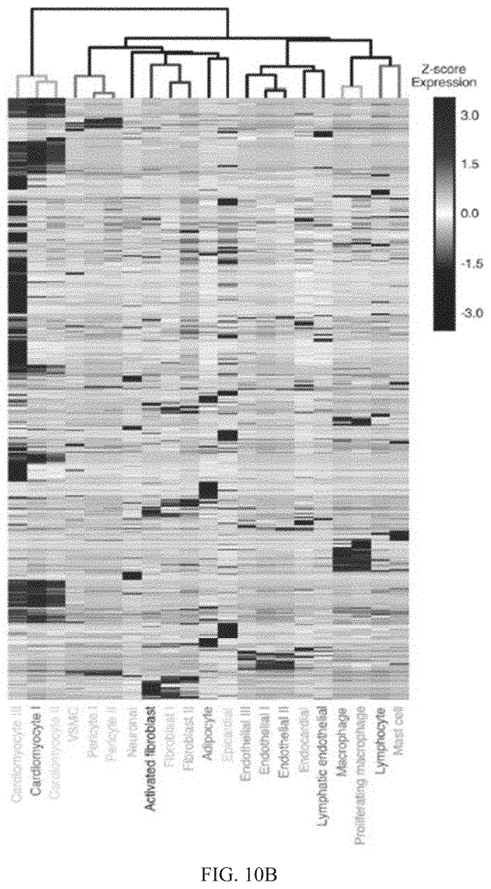

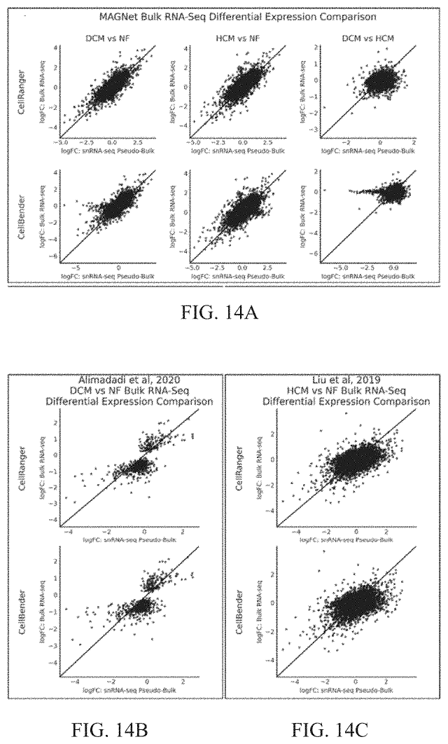

Next, differentially expressed genes between cardiomyopathy and NF hearts were identified across all cell types and within each cell type. Substantial changes in transcription when comparing DCM to NF and HCM to NF were identified, but interestingly there were markedly fewer changes between DCM and HCM, consistent with the PCA results above (FIGS. 2A-2B; FIG. 9; FIGS. 14A-14C). Notably, the largest number of differentially expressed genes between each of the cardiomyopathy groups and NF patients (false discovery rate (FDR)<0.01) were found in fibroblasts, suggesting that the largest transcriptional differences occur in this cell type. When comparing changes in expression between cardiomyopathy and NF patients from bulk RNA sequencing to snRNA-seq, the strongest concordance was observed for pseudo-bulk, cardiomyocytes, and fibroblasts, and less concordance was observed in cell types representing a smaller proportion of the overall milieu of the left ventricle (FIG. 2C). This suggests that bulk RNA sequencing tends to capture differences in gene expression of the more prevalent cell types while snRNA-seq provides more precise information about the cell types driving these differences. There was a small set of genes differentially expressed between DCM and HCM patients, primarily in cardiomyocytes, but these generally showed no patterns for enrichment in particular pathways. Overall, these results suggest a convergence to a common transcriptional profile in advanced cardiomyopathy requiring transplantation, consistent with previous observations based on proteomic analyses.⁴

To further look for transcriptional differences amongst cardiomyopathy patients, HCM samples with preserved ejection fraction were compared to HCM samples with reduced ejection fraction. Differentially expressed genes were not observed in any cell-type at an FDR<0.01. Additionally, in an exploratory analysis, the presence of genes with expression changes in cardiomyopathy that differed dependent on sex were assessed. A set of five genes that showed an interaction (FDR<0.10) between cardiomyopathy status and patient sex, including THRB, encoding Thyroid Hormone Receptor Beta, were identified, suggesting that a small subset of genes may act differently in cardiomyopathy dependent on sex (FIG. 2E).

Based on differential gene expression between cardiomyopathy and NF patients, pathway enrichment analyses were performed by cell type to identify any systematic patterns in dysregulated genes in left ventricle tissue from cardiomyopathy patients (FIG. 2D). In general, pathway enrichments based on DCM compared to NF differential expression and HCM compared to NF differential expression were similar. Some pathways, such as neutrophil degranulation, signaling by interleukins, and metabolism of vitamins and cofactors showed similar enrichments across cell types. Other pathways, such as extracellular matrix organization showed upregulation in cardiomyopathy for some cell types (cardiomyocytes) and downregulation in others (vascular smooth muscle). Of note, cardiomyocytes showed very little systematic up- or down-regulation in cardiomyopathy patients. Conversely, in fibroblasts robust dysregulation in cardiomyopathy of several pathways was observed, including complement cascade, neutrophil degranulation, metabolism of vitamins and cofactors, potassium channels, and neuronal systems. Likewise, in macrophages multiple pathways exhibited dysregulation among myopathic hearts, including neutrophil degranulation, generation of secondary messenger molecules, and co-stimulation of the CD28 family. In addition, genes involved in cytokine and interferon signaling showed upregulation in endothelial cells of cardiomyopathy patients.

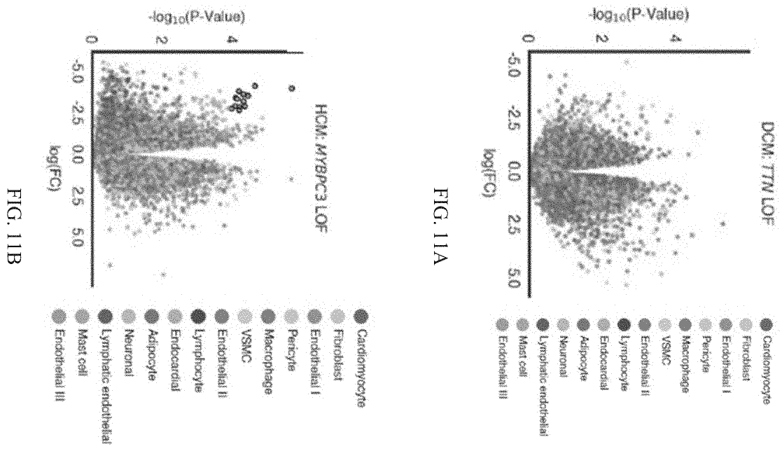

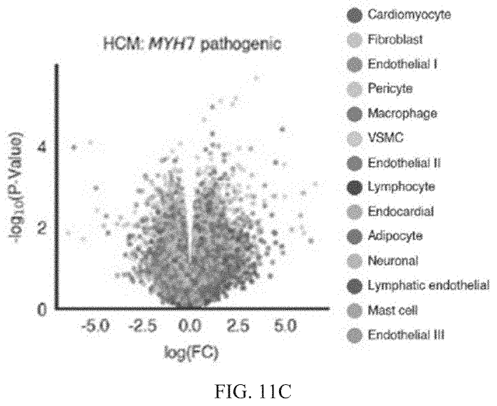

Next, it was determined whether there were any distinct transcriptional differences based on the genetic basis of DCM or HCM. Whole genome sequencing was performed on 40 of the 42 patients included in this analysis to identify loss-of-function (LOF) mutations in known cardiomyopathy genes. The expected enrichments of TTN LOF carriers in DCM (4/10), MYBPC3 LOF carriers in HCM (3/15), and MYH7 pathogenic variant carriers in HCM (5/15) were observed (FIG. 1D). However, there were no appreciable transcriptional differences in TTN LOF carriers compared to other DCM patients, MYBPC3 LOF carriers compared to other HCM patients, or MYH7 pathogenic variant carriers compared to other HCM patients (FIG. 11A-11D).

### Example 3: Distinct Proliferating Macrophage and Activated Fibroblast Populations in Cardiomyopathy Patients

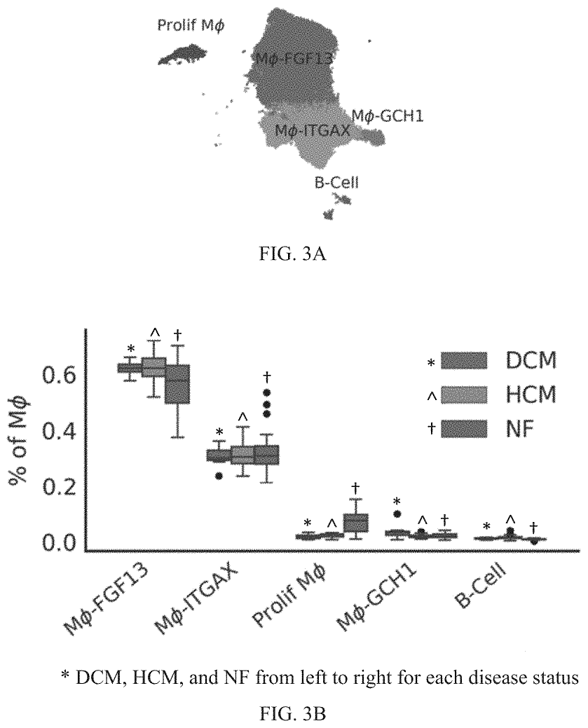

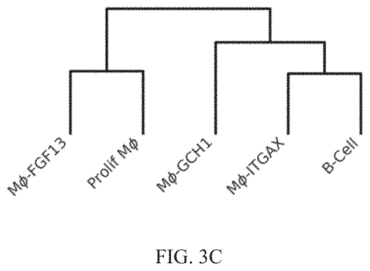

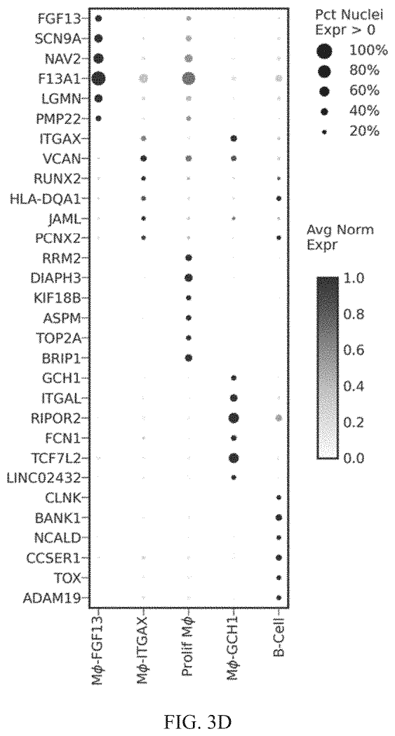

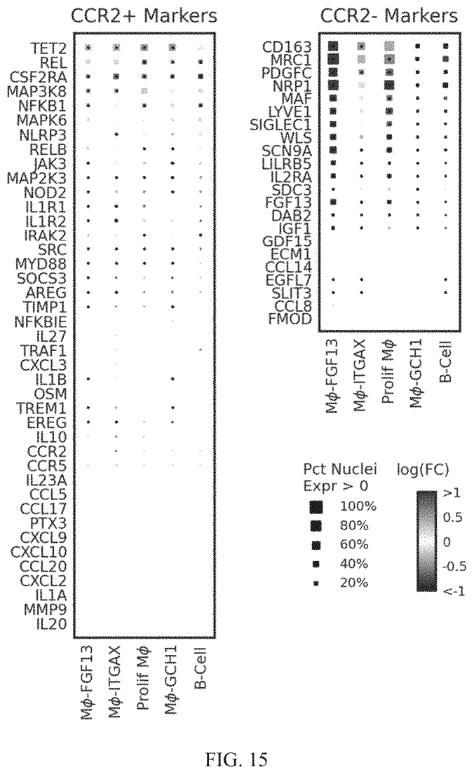

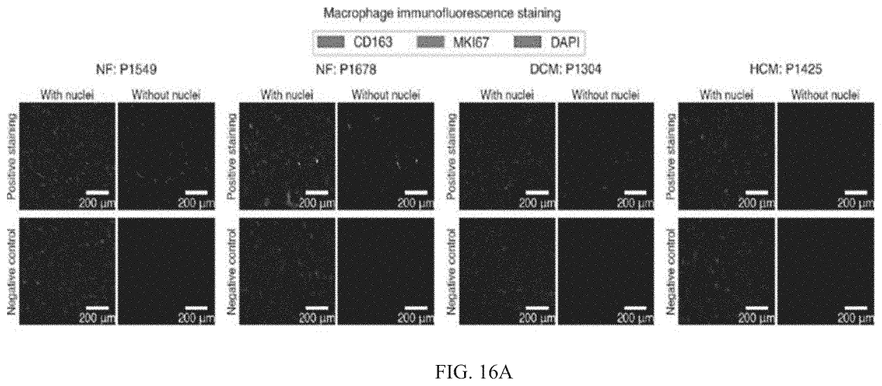

Beyond assessing differential expression between cardiomyopathy and NF left ventricles within given cell types, a skew in distribution of cardiomyopathy and NF nuclei was noted in two cell types. Amongst all macrophages, 5 sub-clusters were identified, including a cluster of proliferating macrophages with upregulation of known cell cycle markers such as RRM2, TOP2A, and BRIP1 (FIG. 3A; FIG. 3D). While most sub-clusters appeared present in similar proportions between NF and cardiomyopathy, proliferating macrophages were notably reduced in patients with a cardiomyopathy (FIG. 3B). This population more closely resembles reparative CCR2 (C-C chemokine receptor 2) negative resident macrophages, as indicated by the elevated expression of prototypical marker genes for this heart macrophage subset (FIG. 3C; FIG. 3D; FIG. 15).

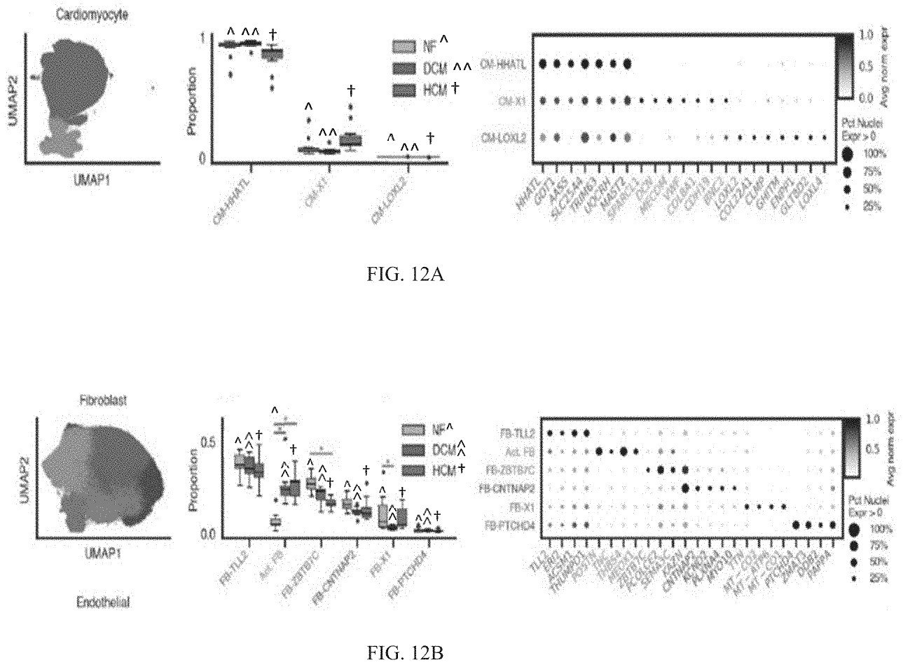

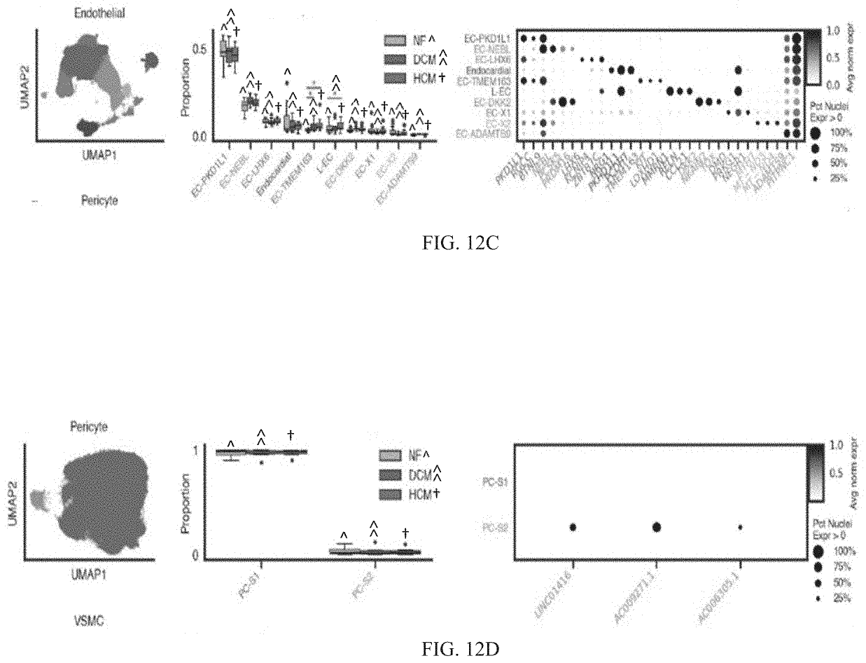

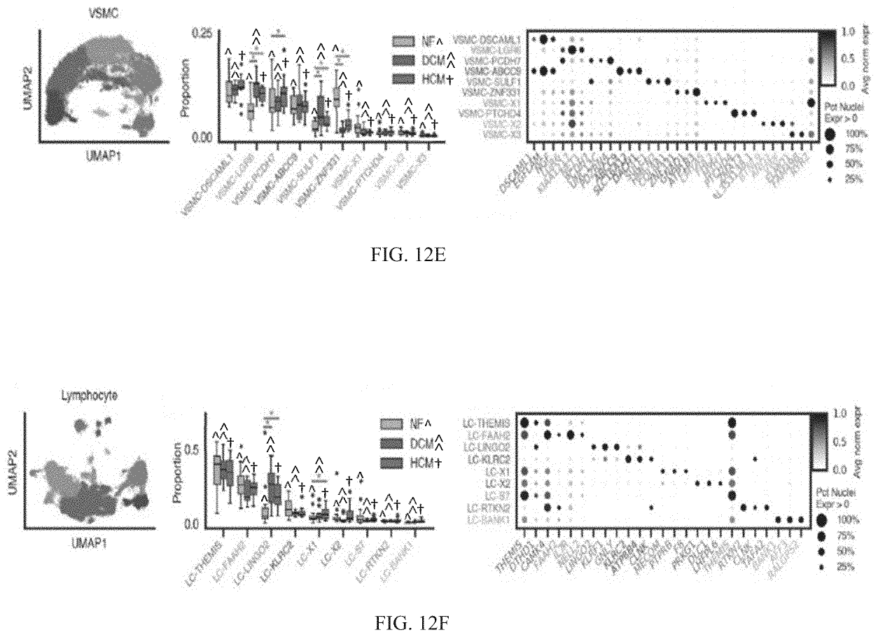

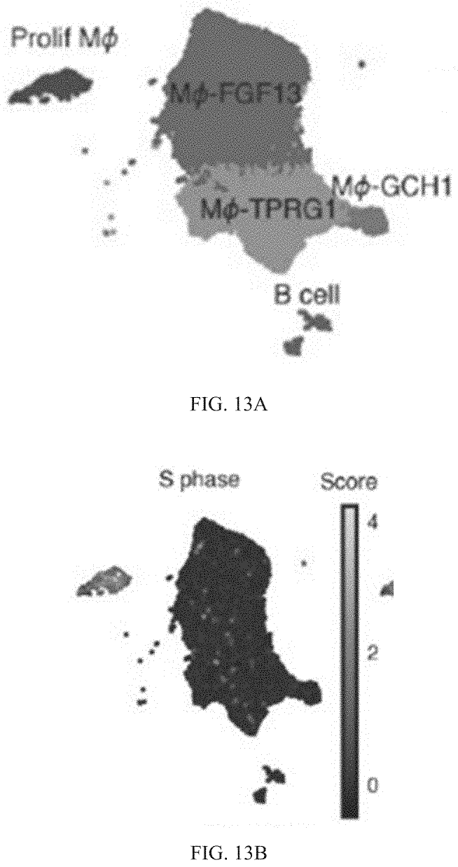

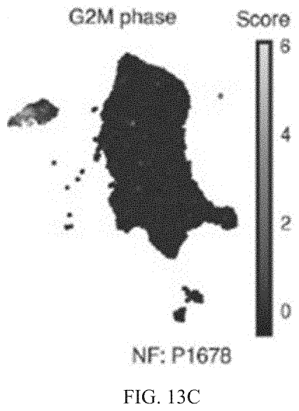

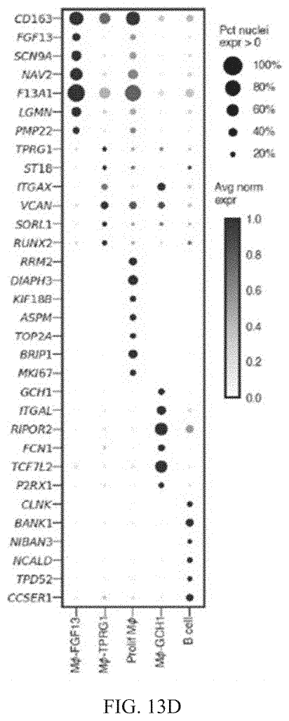

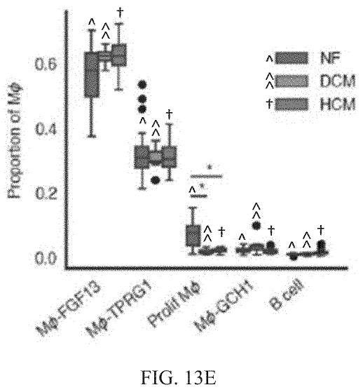

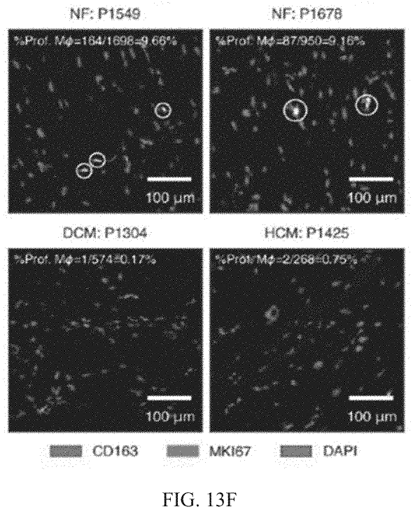

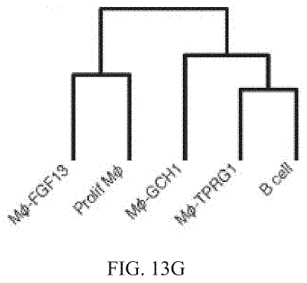

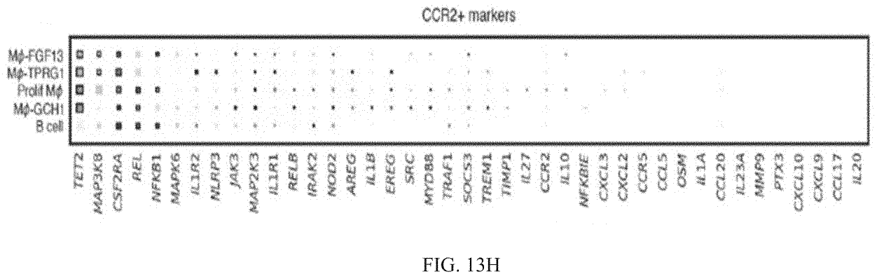

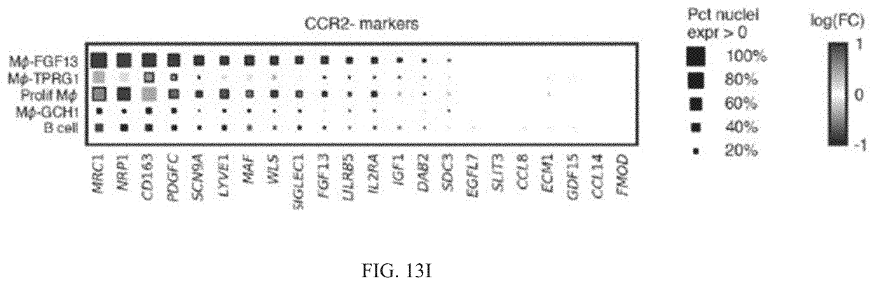

To identify sub-populations within global cell types that may shift in composition in cardiomyopathy, a formal sub-clustering analysis of the most abundant cell types was performed. At least one sub-population with a statistically credible change in composition was observed in either DCM or HCM within fibroblasts, macrophages, VSMCs, and lymphocytes (FIGS. 12A-12F, FIG. 13E). To characterize these sub-populations, upregulated genes for each sub-population were examined compared to the remainder of nuclei from the given cell type (FIGS. 12A-12F, FIG. 13D).

A compositional increase in the endothelial sub-population EC-TMEM163 was observed in both DCM and HCM compared to NF patients (FIG. 12C). This endothelial sub-population displayed increased expression of previously reported angiogenic endothelial markers, including KIT, NRP2, COL15A1, PCDH17, and ITGA6.¹² Similarly, an increase in the lymphocyte sub-population LC-LINGO2 in DCM and HCM was observed compared to NF patients (FIG. 12F). Compared to other lymphocytes, LC-LINGO2 showed increased expression of several known natural killer (NK) cell markers including KLRF1, GNLY, CD244, and PRF1.¹³

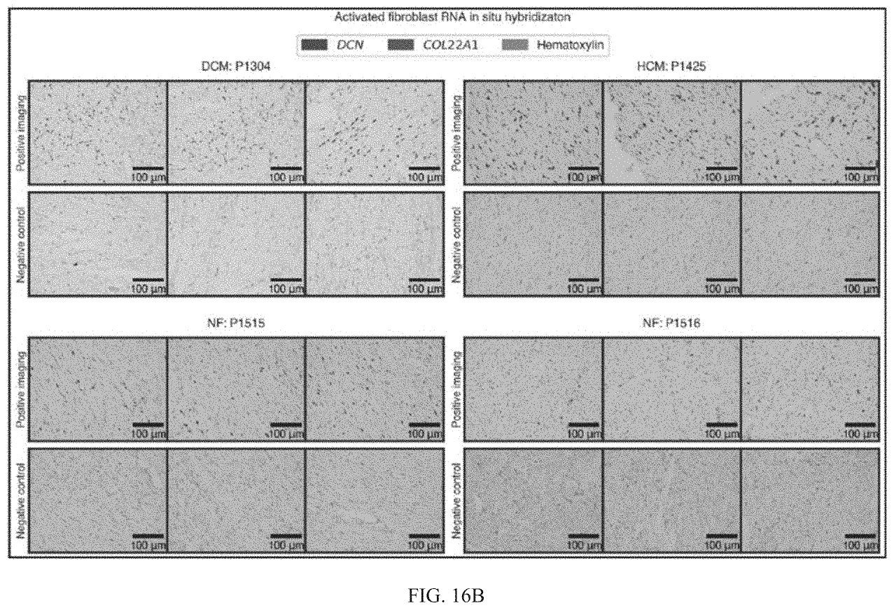

Among the macrophage populations, 4 macrophage sub-populations were identified, including a cluster of proliferating macrophages with a highly enriched cell cycle score and upregulation of many known cell cycle markers (FIGS. 13A-13D).¹⁴ Comparing the composition of macrophage subpopulations across patients, a statistically credible reduction in abundance of proliferating macrophages was observed in patients with a cardiomyopathy (FIG. 13E). Immunofluorescence staining of the macrophage marker CD163 and cell cycle marker MKI67 confirmed the enrichment of these proliferating macrophages in NF patients (FIG. 13F, FIG. 16A). By further interrogating the expression of prototypical marker genes of CCR2 (C-C chemokine receptor 2) positive and CCR2 negative macrophages across these sub-populations, it was observed that this population of proliferating macrophages more closely resembles reparative CCR2 (C-C chemokine receptor 2) negative resident macrophages (FIGS. 13G-I).¹⁵ This observation agrees with the notion that replenishment of CCR2 negative macrophages relies on local proliferation rather than monocyte recruitment.

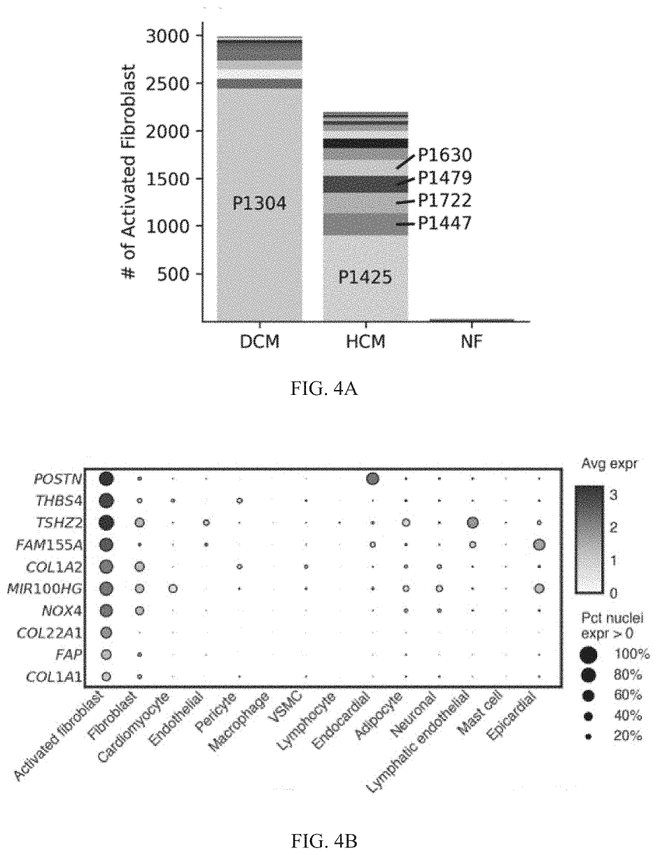

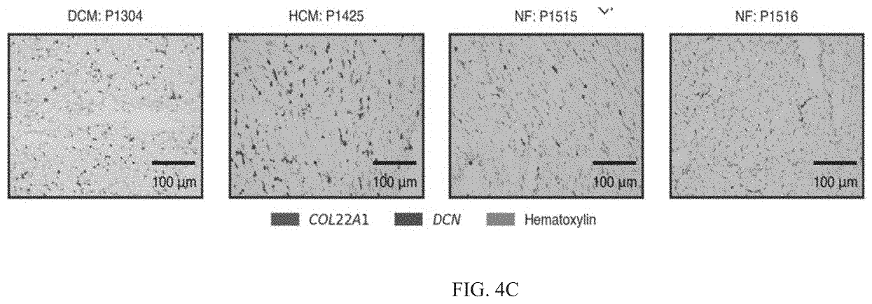

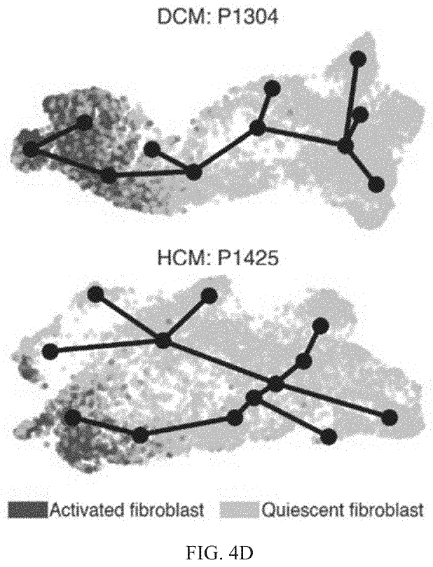

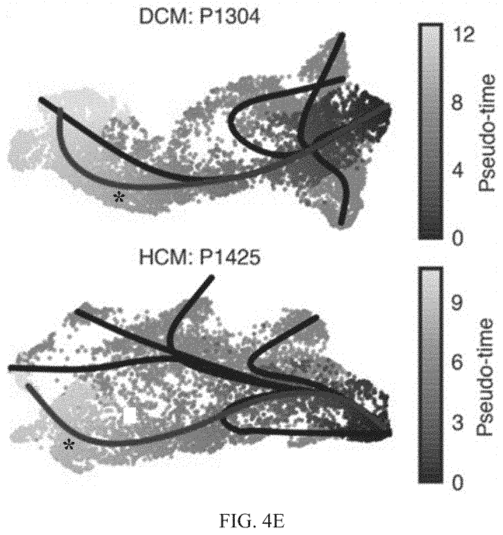

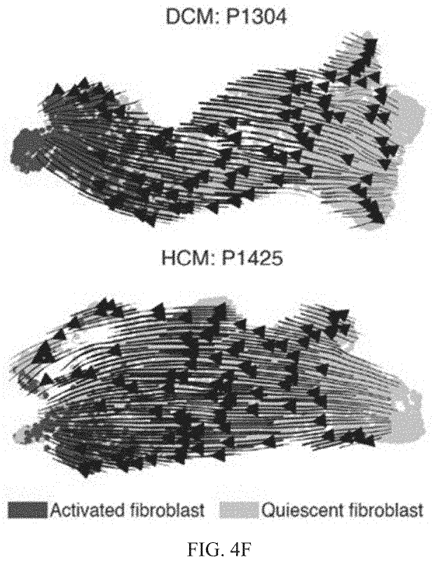

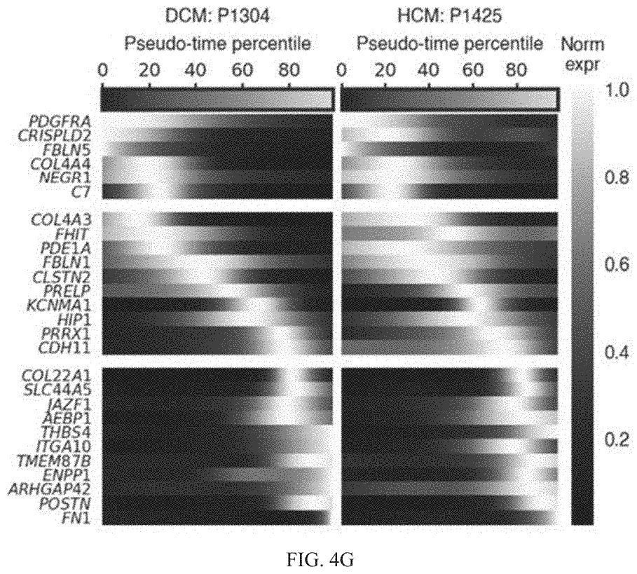

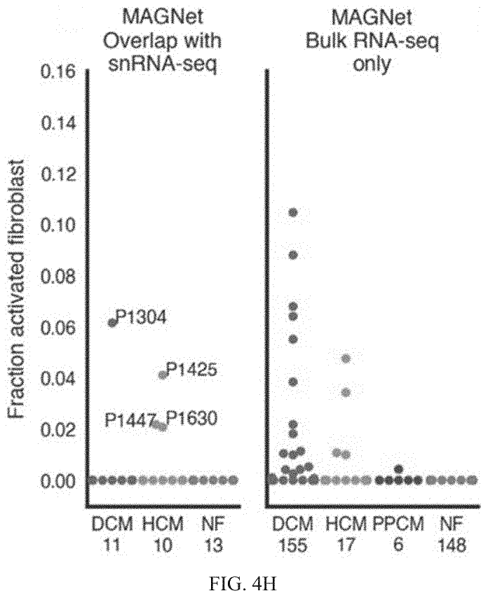

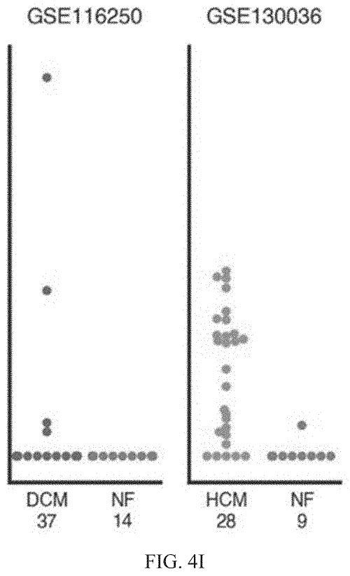

The starkest cell type composition change observed in cardiomyopathy was a cluster of activated fibroblast nuclei nearly exclusively found in cardiomyopathy patients (2989 DCM, 2196 HCM, and 25 NF nuclei), largely derived from DCM patient P1304 and HCM patient P1425 (FIG. 4A). This population showed elevated expression of known activated fibroblast markers such as POSTN, NOX4, FAP, and COL1A1/COL1A2¹⁶⁻¹⁹, as well as previously unreported genes such as Family With Sequence Similarity 155 Member A (FAM155A) and Teashirt Zinc Finger Homeobox 2 (TSHZ2) (FIG. 4B), and had very modest enrichment of the canonical myofibroblast marker ACTA2¹¹ (FIG. 4B). An additional activated fibroblast marker, Thrombospondin-4 (THBS4), has been shown to be up-regulated in matrifibrocytes^{20,21} and involved in myocardial remodeling, interstitial fibrosis, and cardiomyopathy.^{20,22} To validate the presence of this population in diseased patients, RNA in-situ hybridization was performed using the highly specific activated fibroblast marker COL22A1 (log fold change=8.14; adjusted P=3.96×10⁻¹¹²) and the general fibroblast marker DCN. Both patients with the largest activated fibroblast population (P1304 and P1425) showed the presence of COL22A1 expression, which was absent from NF patients (FIGS. 4C and 16B).

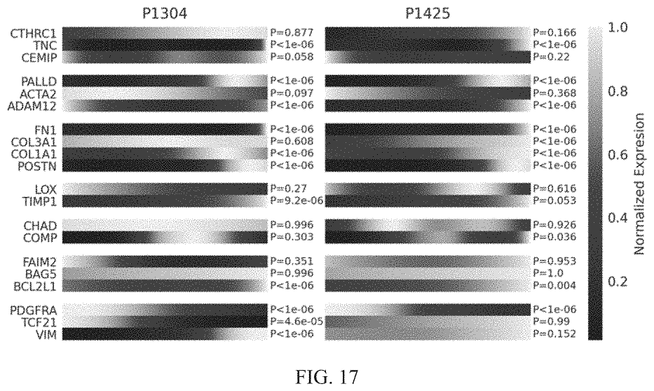

To better understand the gradient of expression between quiescent and activated fibroblasts, a trajectory analysis was performed in two patients with the largest population of activated fibroblasts, P1304 and P1425 (FIGS. 4D-4F). Several genes, including SLC44A5, COL22A1, POSTN, AEBP1, JAZF1, and THBS4 showed increased expression across the trajectory, while other genes such as NEGR1, PDGFRA, C7, FBLN5, and COL4A4 showed decreased expression across the trajectory (FIG. 4G). Notably, a subset of genes including KCNMA1, HIP1, PRRX1, and PRELP showed elevated expression at various stages along the gradient (FIG. 4G). Whether stable intermediate states exist along this trajectory will require further evaluation. Comparing gene expression changes across this inferred gradient to previous work tracing mouse fibroblasts after infarction injury, changes were noted in several genes previously reported (FIG. 17).

To examine whether the presence of activated fibroblasts in cardiomyopathy patients is generalizable to the broader population of patients, an expanded set of patients with DCM, HCM, or peripartum cardiomyopathy, and NF controls from the MAGNet study were used and studied with available bulk RNA sequencing data. Cell type specific expression profiles derived from snRNA-seq data were used to deconvolute bulk RNA sequencing data and estimate a fraction of activated fibroblasts per patient. In 34 samples with both snRNA-seq and bulk RNA sequencing, 4 samples were predicted to have a population of activated fibroblasts in deconvolution analysis, 3 of which had the largest populations in the snRNA-seq data (FIG. 4H).

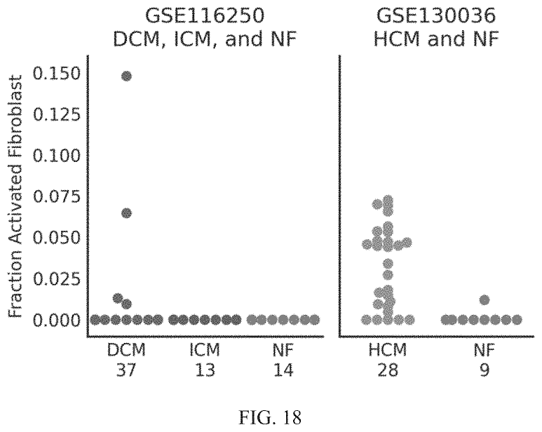

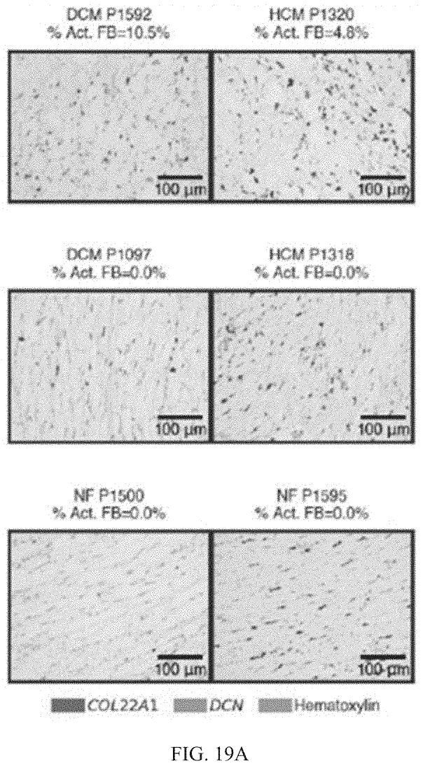

Among the 320 independent patients with bulk RNA sequencing data, 17 of 155 with DCM, 4 of 17 with HCM, and none of the 148 NF control patients were predicted to have activated fibroblasts, confirming the specificity of this population in cardiomyopathy patients (FIG. 4H). The deconvolution analysis was repeated in an independent collection of DCM and NF samples⁶ and predicted an activated fibroblast population in 4 of 37 DCM samples and 0 of 14 NF samples. In a distinct collection of HCM patients undergoing septal myectomy and NF samples,²³ an activated fibroblast population was predicted and observed in 23 of 28 HCM samples and 1 of 9 NF samples (FIGS. 41 and 18). The increased prevalence of the activated fibroblast population earlier in the time course of the disease suggests a pathological role for these cells.

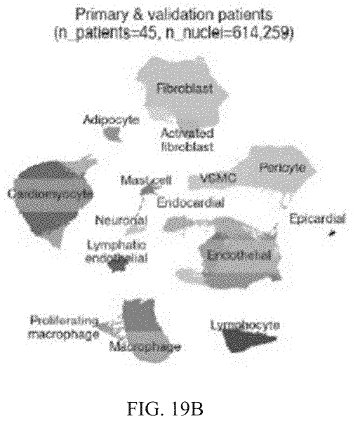

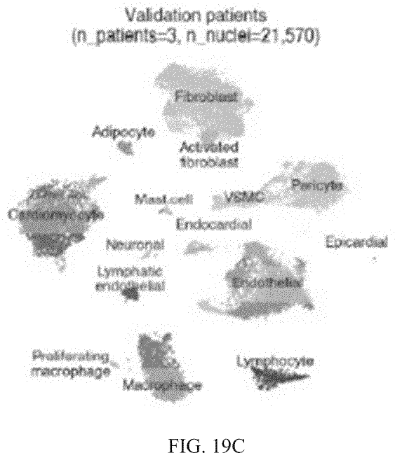

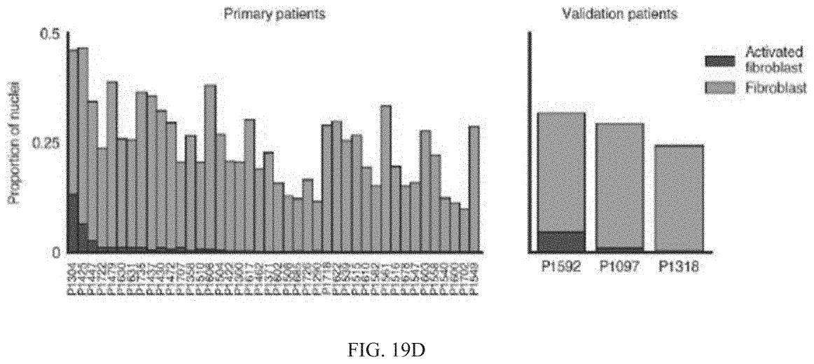

To validate the deconvolution analysis, tissue from 6 additional patients was obtained from the MAGNet study and RNA in-situ hybridization for activated fibroblasts was performed. Two patients predicted to have an activated fibroblast population showed elevated levels of COL22A1, 3 of the 4 patients predicted not to have activated fibroblasts showed little to no COL22A1 expression, and one DCM patient (P1097) with predicted depletion of activated fibroblasts showed modest expression of COL22A1 (FIG. 19A). In addition, snRNA-seq on 3 of these cardiomyopathy patients was performed, and a strong enrichment for activated fibroblasts in 1 sample where an appreciable population of activated fibroblasts in the deconvolution analysis was predicted (FIGS. 19B-19D). In sum, this data suggests that this population of activated fibroblasts is virtually absent from NF hearts, and variably expressed in a subset of patients with DCM and HCM.

### Example 4: Cardiac Fibrosis Assay

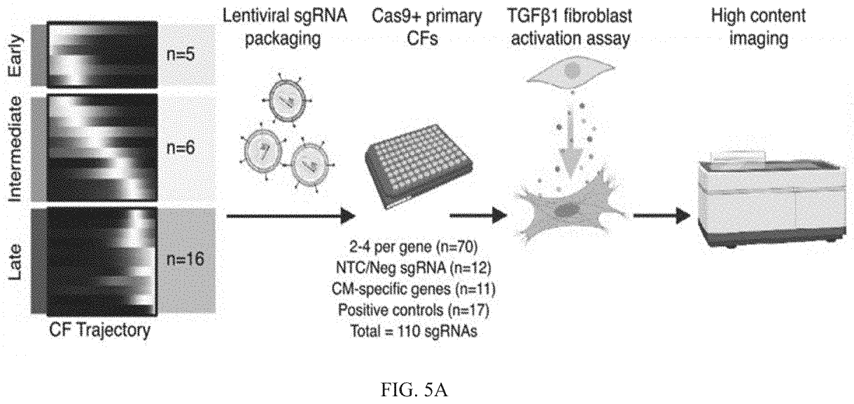

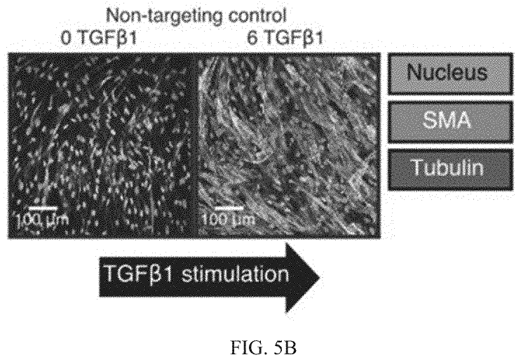

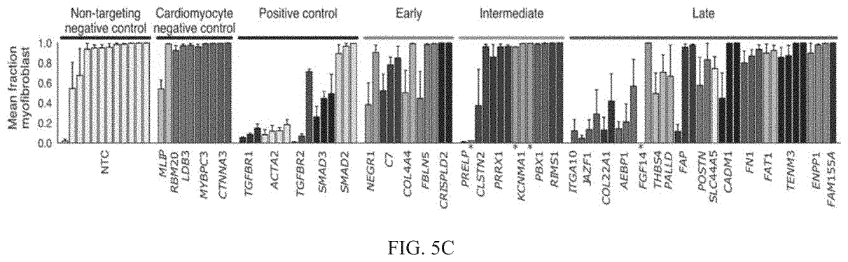

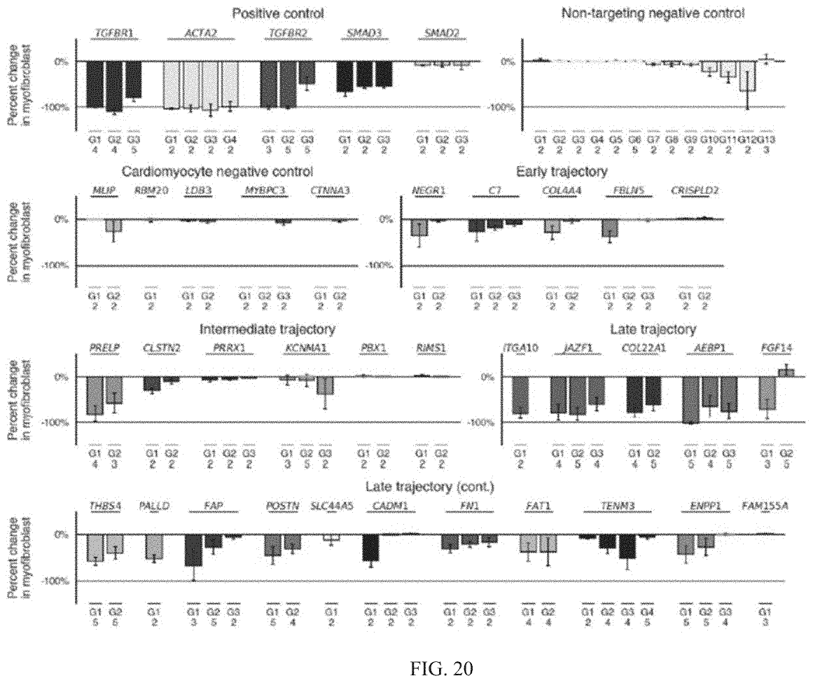

Finally, an assay was developed to measure the progression of cardiac fibroblasts to myofibroblasts upon TGFβ1 stimulation (FIG. 5A). In brief, Cas9+ cardiac fibroblasts were plated on 384-well plates, and 27 genes identified in the activated fibroblast trajectory analysis were knocked out using 2-4 sgRNAs per gene, with each sgRNA replicated across 1-4 wells after filtering. As expected, cardiac fibroblasts treated with TGFβ 1 displayed a stark phenotypic change in smooth muscle actin (SMA), which were used to define the myofibroblast readout (FIG. 5B). As a positive control, several genes known to be involved in TGFβ signaling including ACTA2, TGFBR1, TGFBR2, SMAD2, and SMAD3 were knocked out. A large reduction in the myofibroblast readout for all ACTA2 and TGFBR1 sgRNAs, two of three TGFBR2 sgRNAs, and a lesser reduction for SMAD3 sgRNAs (FIG. 5C) was observed. As negative controls, 12 non-targeting control (NTC) sgRNAs and 5 sgRNAs targeting cardiomyocyte-specific genes were included. Nine of the 12 NTC sgRNAs and all but one sgRNA for the 5 cardiomyocyte-specific genes showed no reduction in the myofibroblast readout post TGFβ1 stimulation (FIG. 5C). At least one sgRNA for 1 gene upregulated in intermediate trajectory fibroblasts and 6 genes upregulated in late stage activated fibroblasts showed a stark reduction (>75%) in myofibroblast readout (FIG. 5C). Each gene from this primary screen was repeated in up to 4 additional screens to assess consistency of results and several sgRNAs showed a consistent large effect across multiple screens, including PRELP, JAZF1, COL22A1, and AEBP1 (FIG. 20). For example, a substantial reduction in SMA for PRELP and JAZF1 was observed compared to positive and negative controls in the primary screen (FIG. 5D). The top performing guides were individually cloned into a Lenti CRISPR v2 backbone, transduced into primary cardiac fibroblasts, and the knockout efficiency was confirmed by real time PCR.

The study permits several conclusions. First, large transcriptional changes were identified at single-cell resolution between DCM or HCM patients compared to NF patients, with relatively few differences between DCM and HCM patients. While there are clear differences in the genetic basis of DCM and HCM, these findings suggest a convergence of these cardiomyopathies to a common transcriptional profile advanced cardiomyopathy requiring transplantation. These results are consistent with previous observations based on proteomic analyses of end stage DCM and HCM left ventricles.⁴

Moreover, compositional shifts in cell types, or cell sub-types, between cardiomyopathy and NF hearts were uncovered. The most prominent shifts included a reduction of proliferating CCR2 negative macrophages in cardiomyopathy hearts and increase in activated fibroblasts in cardiomyopathy hearts. In the case of activated fibroblasts, these nuclei were found nearly exclusively in DCM and HCM patients and computationally predicted to be present in an independent subset of cardiomyopathy patients.

To expand the understanding of cardiac fibroblast activation, several genes with changes in expression along the quiescent to activated fibroblast trajectory were identified in the snRNA-seq data that also showed a reduction in the myofibroblast readout when knocked out in the cellular assay of TGFβ1 induced fibroblast activation. One of the most striking genes in this assay, PRELP, which encodes the extracellular matrix protein Prolargin, may serve as a basement membrane anchor and is known to bind collagen type I and type II, suggesting a plausible role in cardiac fibrosis.²⁴ A second gene, JAZF1, encodes juxtaposed with another zinc finger protein 1, has largely been studied in the context of type 2 diabetes and shown to play a role in lipid and glucose metabolism, as well as insulin sensitivity.²⁵⁻²⁷ COL22A1, encoding Collagen Type XXII Alpha 1 Chain, is expressed at myotendinous junction particularly in skeletal and heart muscle.²⁸ In dermal fibroblasts, silencing COL22A1 impeded the myofibroblast transition as observed by reduced ACTA2, consistent with the observation in cardiac fibroblasts.²⁹

While no substantial difference was observed in the fraction of most macrophage sub-clusters between cardiomyopathy and NF hearts, a notable reduction of proliferating CCR2 negative macrophages in DCM and HCM patients was observed. Mouse models have suggested a loss of resident CCR2 negative macrophages after cardiac injury,²⁰ which may be reflected in this reduction of proliferation in cardiomyopathy patients. Additionally, resident cardiac macrophages have been implicated in preserving cardiac homeostasis through their involvement in both electrical conductance²¹ and elimination of dysfunctional or unnecessary mitochondria from cardiomyocytes,²² further increasing interest in this reduction of proliferation in disease.

Finally, a population of activated fibroblasts in DCM and HCM was identified that was predicted to be present in an independent subset of cardiomyopathy patients. While this population of activated fibroblasts contains several known markers of cardiac activated fibroblasts, such as POSTN, strong enrichment of previously unreported genes including Family With Sequence Similarity 155 Member A (FAM155A), Teashirt Zinc Finger Homeobox 2 (TSHZ2), and AE Binding Protein 1 (AEBP1) was also seen. An additional gene that strongly distinguished activated fibroblasts from quiescent fibroblasts, Thrombospondin-4 (THBS4), has been implicated in myocardial remodeling, interstitial fibrosis, and cardiomyopathy. Although THBS4 been shown to be up-regulated in matrifibrocytes, this activated fibroblast population lacks expression of other known matrifibrocytes markers, including COMP, CHAD, and CILP2. Overall, this comprehensive transcriptional profile expands understanding of the transcriptional diversity within cardiac activated fibroblasts associated with end-stage cardiomyopathies. It is notable that several thousands of individual nuclei from this discovery and validation samples had a similar expression pattern.

Interestingly, a subset of genes with highest expression at an intermediate point on the gradient between quiescent and activated fibroblasts was identified, including KCNMA1, which encodes the alpha subunit of big potassium (BK) channels in cardiac fibroblasts.⁶⁹ KCNMB1, encoding a beta subunit of the BK channel, has been shown to be affiliated with myofibroblast differentiation in pulmonary fibroblasts⁶² and knockdown on KCNMA1 in cardiomyocytes in ischemia-reperfusion models has been linked to increased fibrosis.⁶³ These data suggest that KCNMA1 may play a role in the activation of cardiac fibroblasts. A second gene, PRRX1, showed upregulation prior to the end of the inferred trajectory. PRRX1 encodes the paired-related homeobox 1 and has been implicated in atrial fibrillation based on large-scale genetic studies, suggesting a possible link between atrial fibrillation and fibrosis.^{64,65} PRRX1 has also been shown to act in a positive feedback loop with TWIST1 and TNC to serve as a regulator of fibroblast activation.⁶⁶

In conclusion, the transcriptional landscape at the cellular level from the human heart was comprehensively characterized in healthy patients and in patients with dilated and hypertrophic cardiomyopathy. The results identify both a final common transcriptional pathway and the reduction of proliferating resident cardiac macrophages in cardiomyopathy patients. Finally, a comprehensive transcriptional profile of a disease-specific activated fibroblast population is provided, and mechanistic roles in fibroblast activation for several genes are suggested, expanding the current understanding of these cells in fibrotic disease of the heart. In aggregate, the findings extend understanding of the transcriptional and molecular basis of cardiomyopathies, results that will further inform the pathways and potential therapeutic targets for these morbid cardiac conditions.

### Methods

Data Reporting

No statistical methods were used to predetermine sample size. The experiments were not randomized, and investigators were not blinded to allocation during experiments and outcome assessment.

Human tissue samples: Adult human myocardial samples of European ancestry were collected from deceased organ donors by the Myocardial Applied Genetics Network (MAGNet; www.med.upenn.edu/magnet) as previously described.^{7,30} Donors with dilated cardiomyopathy, hypertrophic cardiomyopathy, or no overt evidence of heart disease were selected. In brief, non-failing samples were obtained from organ donors with no history of heart failure and samples from failing human hearts were obtained from explanted hearts of donors receiving a heart transplant. Hearts were arrested in situ with at least 1 liter of ice-cold crystalloid cardioplegia solution and were transported to the lab in ice-cold cardioplegia solution until cryopreservation (always <4 hours). Transmural cardiac tissue was taken from the left ventricular free wall of a non-infarcted region excluding the septum, and predominantly comprised of the anterior wall midway between the apex and base. Written informed consent for research use of donated tissue was obtained from next of kin in all cases. Research use of tissues were approved by the relevant institutional review boards at the Gift-of-Life Donor Program, the University of Pennsylvania, Massachusetts General Hospital, and the Broad Institute.

Single nucleus RNA-sequencing (snRNA-seq): Single nucleus suspensions were generated as previously described.⁴ In brief, left ventricular tissue (approximately 100 mg) was sectioned at 100 um (Leica CM1950 cryostat), resuspended in 4 mL of ice cold lysis buffer (250 mM sucrose, 25 mM KCl, 0.05% IGEPAL-630, 3 mM MgCl₂, 1 uM DTT, 10 mM Tris pH 8.0), and homogenized using a dounce. Large debris was pelleted at 40×g for 1 min (Beckman Coulter Allegra X-15R swinging bucket centrifuge) and the supernatant was filtered sequentially through a 50 um and 10 um filter (pluriSelect Life Science). The filters were washed with 6 mL of PBS wash buffer (0.01% BSA, 5 mM MgCl₂, PBS) and nuclei were pelleted at 550×g for 5 minutes at 4° C., washed in 6 mL of nuclear wash buffer, and recentrifuged. After removal of wash buffer, nuclei were resuspended in approximately 150 uL of cold nuclear resuspension buffer (Nuclear wash buffer+0.4 U/uL of murine RNAse inhibitor (New England Biolabs)) with gentle trituration, mixed 1:1 with trypan blue, then counted on a hemocytometer. Cells were loaded into the 10× Genomics microfluidic platform (Single cell 3′ solution, v3) for an estimated recovery of 5000 cells per device. Processing of libraries was performed according to manufacturer's instructions with a few modifications. First, nuclei were incubated at 4° C. for 15 minutes after emulsion generation to promote nuclear lysis. Second, the reverse transcription protocol was modified to be 42° C. for 20 minutes then 53° C. for 120 minutes. Libraries were multiplexed at an average of 4 libraries per flow cell on an Illumina Nextseq550 in the Broad Institute's Genomics Platform.

Single nuclei RNA sequencing data processing: Left ventricles from 12 patients with DCM, 16 patients with HCM, and 16 NF hearts underwent snRNA-seq, in replicate. Raw base call files for each sample were de-multiplexed and converted to FASTQ files using the 10× Genomics toolkit CellRanger 4.0.0 (cellranger mkfastq). Prior to count quantification with CellRanger, homopolymer repeats [A₃₀, C₃₀, G₃₀, T₃₀] were trimmed as well as the template switch oligo and its reverse complement [AAGCAGTGGTATCAACGCAGAGTACATGGG (SEQ ID NO: 1), CCCATGTACTCTGCGTTGATACCACTGCTT (SEQ ID NO: 2)] using cutadapt³² with parameters (max_error_rate=0.1, min_overlap=20) and (max_error_rate=0.07, min_overlap=10), respectively. Trimmed reads were aligned to the GRCh38 pre-mRNA human reference (v2020-A) using CellRanger 4.0.0 (cellranger count) with —expect-cells 5000. All other parameters were left as defaults.

Sample selection and quality control: Quality control metrics from cellranger count were inspected for each sample (FIG. 6A). Six samples with <50% of reads in cells, <65% of reads confidently mapping to transcriptome, <90% valid barcodes, or abnormally low Q30 were excluded from downstream analysis. Two additional samples were removed after visually inspecting unique molecular identifier (UMI) decay curves and noting a lack of sufficient ambient plateau for subsequent cell calling and background removal with CellBender remove-background v0.2³², github.com/broadinstitute/CellBender (FIG. 6B). Finally, the distribution of counts from Y-chromosome genes were compared between phenotypically classified males and females. No outliers were identified (FIG. 6C).

Samples passing quality control were processed with the remove-background tool from CellBender v0.2³² to both call non-empty droplets and subtract ambient background RNA contamination from count matrices. The following parameters were used: —expected-cells 5000, —z-dim 100, —total-droplets-included 25000, —epochs 150, —learning_rate le-4, and —fpr 0.01. For 3 of the 80 samples (LV_1539_1, LV_1549_1, and LV_1735_1), —learning_rate was adjusted to 5e-5 to reach training convergence.

The tool scR-Invex (github.com/broadinstitute/scrinvex) was applied to assign reads for each sample to exons, introns, or spanning both. For each cell barcode, the proportion of total exonic reads in the droplet was calculated. Upon performing single nucleus RNA sequencing, an increased proportion of exonic reads may indicate a larger component of cytoplasmic material for a given droplet.

Nuclei map construction: 885,944 nuclei deemed non-empty based on CellBender v0.2 were jointly aggregated. All data processing was done in scanpy v1.6.0³³ unless otherwise noted. First, the top 2000 most highly variable genes were selected using sc.pp.highly variable_genes(flavor=‘seurat_v3’). Count data was then log-normalized by nucleus using sc.pp.normalize(le4) followed by sc.pp.log1p( ). Expression of highly variable genes was scaled to unit variance and zero mean with sc.pp.scaleo. The top 50 principal components were then estimated using sc.tl.pcao. To account for large differences in expression across patients as well as technical batch effects, principal components were aligned with Harmony³⁴ as implemented in harmony-pytorch v0.1.4 (github.com/lilab-bcb/harmony-pytorch) using patient as a batch indicator. A neighborhood graph was constructed using all 50 adjusted PCs with Euclidean distance sc.pp.neighbors(n_neighbors=15). To visualize clusters, Uniform Manifold Approximation and Projection (UMAP) was run on the resulting neighborhood, sc.tl.umap(min_dist=0.2). Leiden clustering at resolution 1.5 was used to cluster nuclei into 47 groups, sc.tl.leiden(resolution=1.5).

Key quality control metrics were calculated for all nuclei including the percent of reads mapped to mitochondrial genes based on CellRanger counts, the proportion of reads mapping to purely exonic regions, entropy as calculated using Bayesian entropy estimation from the ndd python library (pypi.org/project/ndd/1.6.3/), and a predicted doublet score as estimated using Scublet³⁵ per sample based on CellRanger counts. Seven clusters comprised of a total 63,068 droplets were identified as enriched for mitochondrial reads and/or the proportion of exonic reads and depleted for entropy (FIGS. 7A-7F). Nine clusters comprised of 77,534 droplets were identified as enriched for doublet populations (FIGS. 7A-7F). All nuclei from these clusters were subsequently removed.

Additionally, low quality nuclei within each sample and cluster combination were identified by looking for outliers on several measures. Performing this quality control by sample and cluster is important to account for heterogeneity between sample preparations and genuine biological differences in complexity by cell type. In brief, for each combination of sample and cluster, an upper and lower bound were set at the 75th percentile plus 1.5 times the interquartile range (IQR) and the 25th percentile minus 1.5 times IQR, respectively, for total UMI, number of genes (n_gene), entropy, or log(n_gene)*entropy. An upper bound for percent of reads mapped to mitochondrial genes, proportion of exonic reads, or doublet score from Scrublet was set at the 75th percentile plus 1.5 times IQR. If a given sample and cluster combination had fewer than 30 nuclei, distributional outliers were unreliable and therefore hard thresholds for removal were set as total UMI>15000, n_gene>6000, entropy<8, proportion of exonic reads >0.18, doublet score >0.30, and log(n_gene)*entropy >75. A final hard cutoff was set to exclude any nuclei with less than 150 UMI, less than 150 genes, or greater than 5% of reads mapping to mitochondrial genes. In total, this removed 280,630 droplets (FIGS. 6A-6F). Finally, the remaining 605,314 nuclei were re-clustered as previously described prior to nuclei quality control using cosine distance, increasing the number of principal components to 100, and applying Leiden clustering at a resolution of 0.6. In total 21 clusters were identified.

Sub-clustering analysis: Sub-clustering was performed on each cell type to identify remaining low-quality or misclassified nuclei in each cell type and to identify further sub-structure within global cell types. Sub-clustering was performed using an iterative approach on major cell types of the following groups: 1. Cardiomyocyte I, II, and III, 2. Fibroblasts I and II, 3. Endothelial I, II, and III, 4. Pericyte, 5. Macrophage, 6. Vascular Smooth Muscle Cell, 7. Lymphocyte I, 8. Endocardial, 9. Adipocyte, 10. Neuronal, 11. Lymphatic Endothelial, 12. Activated Fibroblast, 13. Lymphocyte II, 14. Proliferating Macrophage, and 15. Epicardial. For each subset, a new neighborhood graph was constructed based on the global Harmony-adjusted principal components. Then, Leiden resolution was increased from 0.05 to 1.0 in increments of 0.05 until a cluster emerged with no genes having area under the receiver operating characteristic curve (AUC)>0.60 in predicting that class.

Sub-clusters of misclassified nuclei were identified by scoring nuclei with sc.tl.score_genes( )¹⁴ based on the top 50, as ranked by AUC, markers of every cell type as well as all mitochondrial genes. Marker genes were identified by calculating a log fold-change and AUC for all genes comparing expression in each cell type to all other cell types. Sub-clusters enriched for scores of other cell types were considered misclassified or low quality. Based on elevated scores, 12,625/605,314 (2.1%) nuclei were identified as potentially low quality or misclassified and removed from subsequent analysis (FIGS. 2A-2E). This included 2802/144,811 (1.9%) fibroblast nuclei, 1797/56,512 (3.2%) macrophage nuclei, 1164/19,301 (6.0%) vascular smooth muscle nuclei, 666/16,912 (3.9%) lymphocyte I nuclei, 1330/7819 (17.0%) endocardial nuclei, 1194/6514 (18.3%) adipocyte nuclei, 1611/5903 (27.3%) neuronal nuclei, 387/5568 (7.0%) lymphatic endothelial nuclei, 241/4706 (5.1%) lymphocyte II nuclei, 1232/2508 (49.1%) proliferating macrophage nuclei, and 201/470 (42.8%) epicardial nuclei (FIGS. 7A-7G).

Cell type identification: A list of genes selectively expressed in each cell type was constructed using a combination of strategies. First, an AUC at a nuclei level was estimated, agnostic of sample by classifying each nucleus as either coming from the target cell type of interest or not, and then predicting this class with the normalized expression values for each gene were used to classify nuclei into each group. Second, a formal differential expression model was run on the aggregation of gene counts for each patient as motivated by Lun and Marioni, 2017.³⁶ For each cell type, gene counts for all nuclei belonging to either technical replicate from a patient were summed together if the total nuclei counts was greater than 50. Genes were filtered out if their counts were too low for testing as determined by the edgeR function filterByExpr(group=cell type).³⁷ DESeq2 normalization³⁸ was applied to estimate scaling factors by sample/cell type combination and then analyzed using the limma-voom pipeline³⁹ with a design of ‘0+cell type+individual’ to account for the fact that individuals have nuclei from multiple cell types and are therefore paired by design. This model was chosen as Lun and Marioni, 2017³⁷ showed the least FDR inflation using summed counts with limma-voom compared to other methods. Contrasts were fit to extract differential expression results for each cell type and two-sided P-values were adjusted with a Benjamini-Hochberg correction.

Marker genes were selected as protein coding genes expressed in at least 25% of nuclei from the target cell type of interest, with an AUC for the target cell type greater than 0.60, a log-fold change from the limma-voom model greater than 2, and an FDR adjusted p-value from the limma-voom model <0.01. Enrichment for biological process gene ontologies for each set of marker genes was performed using the R package GOstats version 2.46.0 with one-sided P-values.⁴⁰ Only gene ontologies with more than 10 genes, but less than 1000 genes, were considered. A significance threshold was set at 0.05/54,009=9.25e-7 to account for 54,009 tested ontologies tested across all cell types. Marker genes and enriched ontologies were used to assign cell type labels to each cluster. Cell type centroids were clustered based on the mean expression of the top 2000 most highly variable genes using Euclidean distance and Ward linkage to display as a dendrogram.

Composition analysis: All composition analyses were performed using the Bayesian method scCODA 0.1.2.post1.41 A default Normal prior was used for intercepts, a spike-and-slab prior for each covariate on a cell type, and a non-centered parameterization of a Normal distribution prior for significant effects. Hamiltonian Monte Carlo was done with 20,000 iterations, using 5,000 iterations as burn-in.

Since compositional analysis is performed relative to a reference cell type, a reference cell type was selected using a method inspired by microbiome work.⁴² In brief, for each cell type i, the standard deviation of the log₁₀ ratio of cell type i counts to cell type j across samples was calculated for each other cell type j. The median of those standard deviations was used, and the cell type with the lowest median was found and was assigned as the reference group. All composition analyses were adjusted for sex.

Differential expression testing: Differential expression testing was performed by cell type. To account for correlation in expression among nuclei from a given individual, counts were summed across nuclei for each patient within each cell type, requiring a minimum of 25 nuclei. A limma-voom model was applied, adjusting for age and sex using the model Gene_Expression˜group+sex+age for each gene. Mitochondrial genes and ribosomal genes were excluded, genes in less than 1% of nuclei in the given cell type of both groups being compared were removed, and an additional filter for lowly expressed genes was applied using the filterByExpr(group=group)³⁷ function. A Benjamini-Hochberg correction was applied for multiple testing correction and an FDR-adjusted p-value<0.01 was used for significance. Differential expression tests were run on both CellBender remove-background and CellRanger counts. The interpretation of genes found was focused on those that significantly differentially expressed both the CellBender and CellRanger counts to ensure robustness in the data presented. To identify genes with a different effect between cardiomyopathy and non-failing patients dependent on sex, a second differential expression test was performed including an interaction term for sex*disease. Due to limited statistical power given the low sample size, a more lenient FDR was set to 0.1 for exploratory analysis.

Flagging potential ambient, background contamination in differential expression results: Generally, in snRNA-seq experiments, some ambient background RNA remains in most droplets despite running CellBender remove-background. To flag genes in each cell type as having a high probability of coming from background RNA, a heuristic was calculated by the following: First, for each gene, two positive predictive values were calculated for each cell type by dichotomizing expression as 0 or >0 (PPV0) and <1 or >1 (PPV1) and predicting cell type class. Second, the mean of PPV0 and PPV1 was calculated. Genes were then flagged as potentially coming from background if 1. A positive predictive value (PPV) for each gene, for each cell type, by dichotomizing expression as 0 or >0 and predicting the cell type class. This is referred to as PPV0; a PPV for each gene as in step 1, but dichotomizing expression as ≤1 and >1. This is referred to as PPV1; the mean of PPV0 and PPV1 for each cell type. This value heuristically represents the probability that if a gene is expressed, it comes from the cell type of interest; and the average log1p(count/total counts+10000) across all nuclei for each cell type.

Genes were then flagged as coming from potential background for a given cell type if they met the following conditions: the maximum mean (PPV0, PPV1) across all cell types minus the mean (PPV0, PPV1) for the given cell type is greater than 0.5, and 2. The average log-normalized expression in the cell type with the maximum mean (PPV0, PPV1) is greater than the given cell type of interest.

Additionally, the interpretation of genes found significantly differentially expressed using both the CellBender and CellRanger counts was focused to ensure robustness in the data presented.

Sample principal component analysis: Principal component analysis (PCA) was performed on counts summed across all nuclei in a patient. Lowly expressed genes, identified as those with less than 10 counts in total, were removed prior to analysis. A variance stabilizing transformation (vst) was applied to counts after estimating the dispersion-mean relation using the R package DESeq2.³⁸ Principal components were estimated using the prcomp function in R v3-5 using the top 500 highly variable genes.

Reactome pathway enrichment: After filtering out genes with expression potentially driven by background contamination as described above, an enrichment test of Reactome pathways⁴³ was performed by cell type and disease comparison using two approaches. First, a gene set enrichment analysis (GSEA) was performed using fgsea v1.16.0⁴⁴ on gene sets with greater than 15 genes but less than 500 genes. Genes were sorted by the t-statistic from the differential expression test using CellBender count data and tested for enrichment in Reactome pathways from MSigDB (www.gsea-msigdb.org/gsea/msigdb). Second, a hypergeometric test of enrichment using ReactomePA was performed to emphasize extreme changes.⁴⁵ Genes were selected with a Benjamini-Hochberg corrected P-value<0.01 based on both CellRanger and CellBender counts and split these into upregulated genes (greater than top 90th percentile of the absolute value of all log fold-changes) for the given cell type or downregulated genes (less than −1*top 90th percentile of the absolute value log fold-changes). Given the number of differentially expressed genes, this second approach allows for emphasis of more extreme differential expression results while also providing a control for large discrepancies between CellBender and CellRanger tests. Pathways enriched in both tests (Benjamini-Hochberg corrected two-sided P-value<0.05 for fgsea and Benjamini-Hochberg corrected one-sided P-value<0.05 for ReactomePA) with consistent directionality were considered robust results.

Bulk RNA sequencing data: Bulk RNA sequencing data from the MAGNet consortium was obtained from Gene Expression Omnibus (GEO) using accession number GSE141910. GEO provided abundances were used for deconvolution analyses. FASTQs from Sequence Read Archive (SRA; accession PRJNA595151) were re-aligned to GENCODE v36 (www.gencodegenes.org/human/)⁶⁷ with salmon v1.4.0⁵² and used as input for differential expression testing as a point of comparison to snRNA-seq differential expression results.

Additional publicly available bulk RNA sequencing data containing DCM, ICM, and NF individuals was obtained from GEO accession number GSE116250 and data containing HCM and NF individuals from GEO accession number GSE130036. Formal differential gene expression testing results from bulk RNA sequencing comparing DCM to NF patients was obtained from Alimidadi et al., 2020⁶⁸ and HCM to NF bulk RNA sequencing data from Liu et al., 2019.²³

Sub-clustering analysis: A targeted sub-clustering analysis was performed on 55,991 macrophages from the global analysis. In brief, 2000 highly variable genes were identified across these macrophages with sc.pp.highly_variable_genes(flavor=‘seurat_v3’). Expression was subset to these 2000 genes and count data was log-normalized with sc.pp.normalize_total(le4) and sc.pp.log1p( ), followed by regressing out the total UMI and percent mitochondrial reads per nucleus with sc.pp.regress_out( ). Count data was scaled with sc.pp.scale( ) followed by PCA (sc.tl.pca( )). To align PCs across biological replicates, Harmony with max_iter_harmony=20 was used. A neighborhood graph was calculated using these corrected PCs with sc.pp.neighbors(n_neighbors=10, n_pcs=50, metric=‘cosine’). Finally, UMAP was run with min_dist=0.2 followed by Leiden clustering at a resolution of 0.2.

Targeted subclustering analysis was performed within the most abundant cell types: cardiomyocytes, fibroblasts, endothelial cells, pericytes, macrophages, vascular smooth muscle cells, and lymphocytes. Clustering was performed as was done in the global map with a reduction to n_neighbors=10 in sc.pp.neighbors to capture more local structure. Additionally, both the percent of mitochondrial reads and total UMI per nucleus prior to PCA calculation were regressed out. Leiden clustering within each cell type was performed at a resolutions between 0.1 and 1.0, at increments at 0.1. When a sub-cluster emerged that had no marker genes with AUC>0.6, clustering was halted, and the previous resolution was retained. In the case of VSMCs, these criteria were altered to require at least 2 genes with AUC>0.6 to avoid over-clustering.

Marker genes were calculated analogously to the global cluster analysis. More liberal criteria were used to define marker genes requiring the gene to be expressed in at least 15% of nuclei in the target sub-cluster, an AUC greater than 0.55, a log-fold change greater than 0, and an FDR two-sided P-value<0.01. Composition analysis was performed using scCODA⁴¹ as described previously to identify sub-populations with varying abundance in disease states. For macrophages, nuclei were scored for cell-cycle profiles using sc.tl.score_genes_cell_cycle( ) with default settings.

Activated fibroblast trajectory analysis: An appreciable activated fibroblast population (>500 nuclei) was identified in 2 patients (P1304, and P1425). 8,798 and 7,164 fibroblast nuclei were extracted for patient P1304 and patient P1425, respectively, and cell-state trajectories between quiescent and activated states within each sample were inferred using Slingshot 1.6.0⁴⁶ in R. Genes with non-zero counts in less than 20 nuclei were removed and the remaining data were normalized with Seurat v3.1.5 using SCTransform⁴⁷, regressing out technical effects from the total number of UMI and the percent of mitochondrial reads. PCA was performed on the top 3000 highly variable genes and ran UMAP on the top 30 principal components using cosine distance, min.dist=0.1, and n.neighbors=15. A low min.dist was set to preserve local structure more accurately. A neighborhood graph (FindNeighbors) was calculated followed by Leiden clustering (FindClusters) at a resolution of 1.0. Slingshot was run on this UMAP space based on the resulting clustering. The most representative trajectory from quiescent to activated fibroblasts was selected by following the most connected pathway across the minimum spanning tree inferred by Slingshot.

In addition to Slingshot, RNA velocity was inferred in these two patients using velocyto 0.17.17⁴⁸ with default settings followed by scVelo 0.2.3.49 As single-nuclei RNA-sequencing was performed, the spliced reads in P1304 were limited to 17% and 16% spliced reads in P1425. Genes with less than 20 spliced and unspliced counts were filtered out, and the top 3500 most variable genes were retained. The first and second-order moments for each cell based on 30 nearest neighbors were computed, obtaining the neighborhood graph from Euclidean distance of the top 30 principal components from PCA. The dynamical model was then run with default settings.

To identify interesting gene expression patterns along the trajectory, the method was applied using tradeSeq.⁵⁰ A generalized additive model (GAM) was fit for each gene using fitGAM(nknots=6) on SCTransform adjusted counts. Genes that showed associations with Slingshot inferred pseudo-time were sought using both startVsEndTest( ) and associationTest( ) in tradeSeq. Strong markers of the early trajectory were identified as those with an FDR adjusted two-sided P-value<0.05 from startVsEndTest( ), log fold-change<−1, and found in at least 30% of nuclei at the earliest cluster of the trajectory. Strong markers of the late trajectory were identified as those with an FDR adjusted two-sided P-value<0.05 from startVsEndTest( ), log fold-change>1 and found in at least 30% of the latest cluster of the trajectory. Genes showing other interesting patterns were identified as having an FDR adjusted two-sided P-value<0.05 in associationTest( ) and being most highly expressed in a cluster other than the start or end cluster with a minimum of 30% of nuclei with non-zero expression. Genes were then subset that showed consistent patterns across P1304 and P1425 for visualization.

Activated fibroblast deconvolution analysis: CIBERSORTx⁵¹ (cibersortx.stanford.edu) was applied to computationally deconvolute bulk RNA sequencing experiments and estimate a fraction of activated fibroblasts. Cell type signatures were derived from the global map after collapsing subgroups into broad cell types, including cardiomyocytes, fibroblasts, endothelial, pericyte, macrophage, vascular smooth muscle, lymphocyte, endocardial, adipocyte, neuronal, lymphatic endothelial, activated fibroblast, and epicardial. Markers for each cell type were selected as those in at least 30% of the target cluster at non-zero levels, AUC>0.60, and significant in the limma-voom model with FDR<0.01 and log fold-change>2. A max of 200 genes was selected for each cell type, retaining those with the highest log fold-change estimate.

Bulk RNA sequencing gene abundance data from the MAGNet study containing DCM, HCM, PPCM (peripartum cardiomyopathy) and NF patients was downloaded from GEO (accession ID: GSE141910). Publicly available bulk RNA sequencing data from additional DCM, ICM, and NF samples was obtained from GEO (accession ID: GSE116250) and SRA (accession ID: PRJNA477855). FASTQs were aligned to GENCODE v36 with salmon v1.4.0⁵² to quantify transcripts per million (TPM) as input to CIBERSORTx. Finally, publicly available bulk RNA sequencing data from HCM and NF patients as reported in Liu et al., 2019²³ was obtained from GEO using accession number GSE130036 and used as input to CIBERSORTx.

Whole-Genome Sequencing Analysis: Whole-genome sequencing was performed targeting 30× coverage at the Broad Institute of Harvard and the Massachusetts Institute of Technology using the Illumina NovaSeq platform. Reads were aligned to the GRCh38 reference using BWA-MEM.⁵³ Variants were called using GATK HaplotypeCaller v3.5.0.⁵⁴ In total, 10 of 11 DCM patients, 15 of 15 HCM patients, and 15 of 16 NF patients were successfully sequenced. All samples had mean coverage >25× and <5% chimeras. Variants in 106 genes used in clinical testing panels for cardiomyopathy (Invitae Cardiomyopathy Comprehensive Panel) were annotated for predicted loss-of-function variation (LOF) using the LOFTEE plugin (github.com/konradjk/loftee) for the Ensembl Variant Effect Predictor⁵⁵ in Hail (github.com/hail-is/hail). Insertions or deletions were removed with QD≤3 or single-nucleotide polymorphisms with QD≤2, variants with a failing VQSR flag, variants with a LowQual flag, variants with an InbreedingCoeff flag, variants within low complexity region, and variants in segmental duplications. Only high-confidence LOF variants in canonical transcripts without the LOFTEE flags PHYLOCSF_WEAK or NON_CAN_SPLICE were considered when deeming a sample a LOF carrier. Additional pathogenic variants were identified by looking for pathogenic, or likely pathogenic, missense variants in ClinVar (downloaded Dec. 12, 2020).

Activated fibroblast RNAscope Validation: RNA in situ hybridization was performed as previously described⁷ using RNAscope 2.5 duplex assay (ACD bio). Briefly, fresh frozen samples were sectioned at 10 μm and mounted onto Superfrost plus slides (VWR). Samples were fixed in 4% PFA for 30 min at room temp and treated with protease IV for 30 min. The RNAscope assay was then carried out using the manufacturer's protocol (ACD bio). Probes for Col22A1 (Channel 2) and DCN (Channel 1) were purchased from ACD bio and samples were imaged on a Zeiss Observer Z1 microscope.

Macrophage imaging: Left Ventricular sections (10 um) were fixed (4% PFA, 15 min), permeabilized (0.1% TX-100, 5 min), blocked (7% donkey serum, 1 h) and incubated at 4 degrees overnight with primary antibodies (Ki67 (Abcam), CD163 (Abcam)). The following day, sections were washed 3× in PBS and incubated with secondary antibodies and DAPI in the dark for 1 h at RT (alexa-fluor-488, 568, Thermofisher). Samples were mounted with prolong gold (Thermofisher) and representative images were taken at 20× using a Leica SP8 confocal. For cell counting, tiled images were taken of the whole section at 10× and macrophages and Ki67 positive macrophages were counted using ImageJ.⁵⁶

Cardiacfibrosis assay: Stable Cas9-expressing fibroblasts were generated from primary human cardiac fibroblasts (ACBRI5118, Cell Systems). All sgRNA lentiviral vectors were obtained from the Broad Genetic Perturbation Platform (GPP). Cells were cultured in Lonza FGM-3 Cardiac Fibroblast Growth Medium (CC-4525). To perform the CRISPR-Cas9 gene knock-out screen, on Day 1 Cas9-expressing fibroblasts were seeded at 4,000 cells/well in 50 ul complete growth media containing 10 ug/ml Polybrene (Sigma TR-1003-G) in 384-well Perkin Elmer Cell Carrier imaging microplates. This was immediately followed by transduction with 8 ul/well of lentiviral sgRNA. Negative controls included non-targeting guides and positive controls included TGFBR1, TGFBR2, ACTA2, SMAD2, and SMAD3 guides. Two to four different guides were used per gene. The following day, Day 2, cells were washed twice with 80 ul/well PBS and 60 ul/well selection media containing 2 ug/ml puromycin was then added to the plates.

Transduction and washing of 384-well plates were accomplished using an Integra Viaflo pipetting system. Cells were incubated in selection media for 6 days to ensure complete gene knockout before performing the fibroblast activation assay. On Day 8, the assay was initiated by performing a media exchange for starvation media (CC-4525 without FBS and supplements) containing TGFβ1 (Sigma T7039) at 6 ng/ml (EC99). Cells were stimulated with TGFβ1 for 48 hours to allow fibroblast-myofibroblast transition to occur.

Fixation and immuno-fluorescence staining of plates for imaging was carried out using a Thermo Fisher Multidrop Combi and a Biotek plate-washer. After 48 hours, cells were fixed with 40 ul/well ice-cold 100% Methanol for 20 min and additionally permeabilized with 40 ul/well 0.5% Triton-X 100 in PBS for 15 min. Roche Blocking Reagent at 40 ul/well was used to block cells for 15 min before addition of antibodies. The primary antibody cocktail consisted of Mouse monoclonal [1A4] to alpha smooth muscle Actin (Abcam ab7817/1:500), Goat polyclonal to beta Tubulin (Abcam ab21057/1:500) and COL1A1 (E6A8E) rabbit mAb (Cell Signaling Technology 39952S/1:200). The primary antibody cocktail was incubated with cells at 30 ul/well for 1.5 hours at room temperature. The secondary antibody cocktail consisted of Donkey anti-Goat IgG (H+L) Highly Cross-Adsorbed Secondary Antibody, Alexa Fluor Plus 647 (Life Technologies A32849), Donkey anti-Mouse IgG (H+L) Highly Cross-Adsorbed Secondary Antibody, Alexa Fluor 488 (Life Technologies A21202), Donkey anti-Rabbit IgG (H+L) Highly Cross-Adsorbed Secondary Antibody, Alexa Fluor 568 (Life Technologies A10042) and Hoechst 33342 (Thermo Fisher 62249). All secondary antibodies were used at a 1:1000 dilution and incubated with cells at 30 ul/well RT for 45 min. Two washes of 80 ul/well PBS were used after each addition of fixation and permeabilization, and four washes of 80 ul/well PBS were used after each antibody addition.

Plates were imaged on a laser-powered Perkin Elmer Opera Phenix confocal imaging system using a water-immersion 20× objective with 9 fields of view per well. Image analysis was performed using PerkinElmer Harmony High-Content Imaging and Analysis Software. Single cells were segmented using Hoechst channel for nucleus and Tubulin channel for cytoplasm. To quantify the myofibroblast phenotype, Multiple intensity, STAR morphology and texture parameters for Tubulin and SMA staining were measured within every cell. The “PhenoLOGIC™ Machine Learning Linear Classifier” method (Perkin Elmer) was used to differentiate between fibroblasts and activated myofibroblasts. The control plate, in which the cells were seeded with different cell numbers and treated with different concentrations of TGFB1, was used for training. High concentration wells represented myofibroblasts and low or no TGFB1 wells represented fibroblasts. As a readout, the fraction of myofibroblasts per well was calculated. For each guide, the mean value and standard error was calculated excluding wells with low cell counts. This low cell count cutoff per well was determined by plotting the binned cell number against the fraction of myofibroblasts per well and cell counts for which the control wells did not perform well, were accepted as not reliable. The assay was repeated up to a maximum of 5 times for each gene in the primary screen. Only sgRNAs with more than 1 well of data from at least two replicates are displayed. To compare across screens, well values for the fraction of myofibroblasts were normalized to both the median of all TGFBR1 sgRNAs (sgRNATGFBR1) and the median of all non-targeting control sgRNAs (sgRNANTC), calculated as 100*(fraction of myofibroblast−median(sgRNANTC))/(median(sgRNANTC)−median(sgRNATGFBR1)). An inverse analysis-variance weighted random effects meta-analysis was then perfomed across all runs of the screen to quantify the mean normalized effect for each sgRNA using the metafor package in R.

The top performing sgRNAs for JAZF1 (BRDN0001495689), PRELP (BRDN0003485480), and TGFBR1 (BRDN0000579830) were cloned into the pLentiCRISPR eSpCas9 v2 vector (SC1823, GenScript). Lentiviral particles were generated in HEK293t/17 cells (ATCC) using Lenti-X packaging single shots (Takara) according to the manufacturer's instructions. ACBRI5118 cells were transduced with the lentiviral particles in replicate, and then selected for with Puromycin the day following transduction. RNA was harvested from transduced cells using the RNeasy Mini Kit (QIAgen; cat. No. 74104) with on-column DNA digestion (The RNAse-Free DNase Set; cat. no. 79254) 500 ng of RNA were reverse transcribed into cDNA using iScript cDNA synthesis kit (BioRad; cat. No. 1708890). PrimeTime (Integrated DNA Technologies) forward and reverse primers (sequences in Supplemental Table 14) were used in qPCR reaction mixtures by adding all required components according to the manufacturer's instructions (iTaq Universal SYBR Green Supermix; BioRad; 1725121) on a BioRad CFX384 Real-Time System.

### Example 5: Marker Genes that Identify the Activated Fibroblast Population and Mediator Genes of the Activated Fibroblast Population

Genes that identify the activated fibroblast population (marker genes): Genes were selected based on the following criteria—1. Protein coding genes; 2. Expressed in at least 30% of activated fibroblasts; 3. AUC>0.60; 4. logFC>3.0; and 5. FDR-adjusted p-value<0.01. Using these criteria in mind, 113 marker genes were identified. Using a stricter cutoff of logFC>5.0, this reduces the list to 31 genes, which overlap with the genes below, identified as potential mediators of the activated fibroblast population.

Genes that had a measurable effect in the fibroblast activation assay (mediator genes): A criteria of genes with at least a 50% reduction for one guide in the primary screen was used. Positive Controls: included TGFBR1, TGFBR2, ACTA2, and SMAD3. Genes with a specific effect in the assay, identified as potential mediator genes of the activated fibroblast population, were a) Early Traj Genes: NEGR1, FBLN5, b) Intermediate Traj. Genes: PRELP, CLSTN2, and c) Late Traj. Genes: ITGA10, JAZF1, COL22A1, AEBP1, FGF14, THBS4, FAP, and CADM1.

### REFERENCES

1. Savarese, G. & Lund, L. H. Global Public Health Burden of Heart Failure. Card. Fail. Rev. 03, 7 (2017).
2. Oktay, A. A. et al. Diabetes, Cardiomyopathy, and Heart Failure. Endotext (MDText.com, Inc., 2000).
3. Liu, Y. et al. RNA-Seq identifies novel myocardial gene expression signatures of heartfailure. Genomics 105, 83-89 (2015).
4. Chen, C. Y. et al. Suppression of detyrosinated microtubules improves cardiomyocyte function in human heart failure. Nat. Med. 24, 1225-1233 (2018).
5. Maron, B. J. et al. Contemporary definitions and classification of the cardiomyopathies: An American Heart Association Scientific Statement from the Council on Clinical Cardiology, Heart Failure and Transplantation Committee; Quality of Care and Outcomes Research and Functional Genomics and Translational Biology Interdisciplinary Working Groups; and Council on Epidemiology and Prevention. Circulation 113, 1807-1816 (2006).
6. Sweet, M. E. et al. Transcriptome analysis of human heart failure reveals dysregulated celladhesion in dilated cardiomyopathy and activated immune pathways in ischemic heart failure. BMC Genomics 19, (2018).
7. Tucker, N. R. et al. Transcriptional and Cellular Diversity of the Human Heart. Circulation 142, 466-482 (2020).
8. Litvin̆uková, M. et al. Cells of the adult human heart. Nature 588, 466-472 (2020).
9. Wang, L. et al. Single-cell reconstruction of the adult human heart during heart failure and recovery reveals the cellular landscape underlying cardiac function. Nat. Cell Biol. 22, 108-119 (2020).
10. Ashburner, M. et al. Gene ontology: Tool for the unification of biology. Nat. Genet. 25, 25-29 (2000).
11. Carbon, S. et al. The Gene Ontology Resource: 20 years and still GOing strong. Nucleic Acids Res. 47, D330-D338 (2019).
12. Kalucka, J. et al. Single-Cell Transcriptome Atlas of Murine Endothelial Cells. Cell 180,764-779.e20 (2020).
13. Crinier, A. et al. High-Dimensional Single-Cell Analysis Identifies Organ-Specific Signatures and Conserved NK Cell Subsets in Humans and Mice. Immunity 49, 971-986.e5 (2018).
14. Tirosh, I. et al. Dissecting the multicellular ecosystem of metastatic melanoma by single-cell RNA-seq. Science 352, 189-196 (2016).
15. Bajpai, G. et al. The human heart contains distinct macrophage subsets with divergent origins and functions. Nat. Med. 24, 1234-1245 (2018).
16. Tallquist, M. D. & Molkentin, J. D. Redefining the identity of cardiac fibroblasts. Nat. Rev. Cardiol. 14, 484-491 (2017).
17. Cucoranu, I. et al. NAD(P)H oxidase 4 mediates transforming growth factor-β1-induced differentiation of cardiac fibroblasts into myofibroblasts. Circ. Res. 97, 900-907 (2005).
18. Tillmanns, J. et al. Fibroblast activation protein alpha expression identifies activated fibroblasts after myocardial infarction. J. Mol. Cell. Cardiol. 87, 194-203 (2015).
19. Shinde, A. V. & Frangogiannis, N. G. Mechanisms of Fibroblast Activation in the Remodeling Myocardium. Curr. Pathobiol. Rep. 5, 145-152 (2017).
20. McLellan, M. A. et al. High-Resolution Transcriptomic Profiling of the Heart During Chronic Stress Reveals Cellular Drivers of Cardiac Fibrosis and Hypertrophy. Circulation 142, 1448-1463 (2020).
21. Forte, E. et al. Dynamic Interstitial Cell Response during Myocardial Infarction Predicts Resilience to Rupture in Genetically Diverse Mice. 30, 3149-3163.e6 (2020).
22. Frolova, E. G. et al. Thrombospondin-4 regulates fibrosis and remodeling of the myocardium in response to pressure overload. FASEB J. 26, 2363-2373 (2012).
23. Liu, X. et al. Long non-coding and coding RNA profiling using strand-specific RNA-seqin human hypertrophic cardiomyopathy. Sci. Data 6, (2019).
24. Bengtsson, E. et al. The Leucine-rich Repeat Protein PRELP Binds Perlecan and Collagens and May Function as a Basement Membrane Anchor. J. Biol. Chem. 277, 15061-15068 (2002).
25. L, L. et al. The role of JAZF1 on lipid metabolism and related genes in vitro. Metabolism. 60, 523-530 (2011).
26. GF, M. et al. JAZF1 can regulate the expression of lipid metabolic genes and inhibit lipid accumulation in adipocytes. Biochem. Biophys. Res. Commun. 445, 673-680 (2014).
27. Yuan, L. et al. Transcription factor TIP27 regulates glucose homeostasis and insulin sensitivity in a PI3-kinase/Akt-dependent manner in mice. Int. J. Obes. 39, 949-958 (2015).
28. Koch, M. et al. A Novel Marker of Tissue Junctions, Collagen XXII. J. Biol. Chem. 279, 22514 (2004).
29. Watanabe, T. et al. A Human Skin Model Recapitulates Systemic Sclerosis Dermal Fibrosis and Identifies COL22A1 as a TGFβ Early Response Gene that Mediates Fibroblast to Myofibroblast Transition. Genes (Basel). 10, (2019).
30. Ma, Y. et al. Cardiomyocyte d-dopachrome tautomerase protects against heart failure. JCI Insight 4, (2019).
31. Martin, M. Cutadapt removes adapter sequences from high-throughput sequencing reads. EMBnet.journal 17, 10 (2011).
32. Fleming, S. J., Marioni, J. C. & Babadi, M. CellBender remove-background: A deep generative model for unsupervised removal of background noise from scRNA-seq datasets. Preprint at doi.org/10.1101/791699 (2019).
33. Wolf, F. A., Angerer, P. & Theis, F. J. SCANPY: Large-scale single-cell gene expression data analysis. Genome Biol. 19, 15 (2018).
34. Korsunsky, I. et al. Fast, sensitive and accurate integration of single-cell data with Harmony. Nat. Methods 16, 1289-1296 (2019).
35. Wolock, S. L., Lopez, R. & Klein, A. M. Scrublet: Computational Identification of Cell Doublets in Single-Cell Transcriptomic Data. Cell Syst. 8, 281-291.e9 (2019).
36. Lun, A. T. L. & Marioni, J. C. Overcoming confounding plate effects in differential expression analyses of single-cell RNA-seq data. Biostatistics 18, 451-464 (2017).
37. Chen, Y., Lun, A. T. L. & Smyth, G. K. From reads to genes to pathways: Differential expression analysis of RNA-Seq experiments using Rsubread and the edgeR quasi-likelihood pipeline. F1000 Research 5, 1438 (2016).
38. Love, M. I., Huber, W. & Anders, S. Moderated estimation of fold change and dispersion for RNA-seq data with DESeq2. Genome Biol. 15, 550 (2014).
39. Law, C. W., Chen, Y., Shi, W. & Smyth, G. K. Voom: Precision weights unlock linear model analysis tools for RNA-seq read counts. Genome Biol. 15, R29 (2014).
40. Falcon, S. & Gentleman, R. Using GOstats to test gene lists for GO term association. Bioinformatics 23, 257-258 (2007).
41. Buttner, M., Ostner, J., Müller, C. L., Theis, F. J. & Schubert, B. scCODA: A Bayesianmodel for compositional single-cell data analysis. Preprint at doi.org/10.1101/2020.12.14.422688 (2020).
42. Brill, B., Amir, A. & Heller, R. Testing for differential abundance in compositional countsdata, with application to microbiome studies. Preprint at arxiv.org/abs/1904.08937 (2019).
43. Jassal, B. et al. The reactome pathway knowledgebase. Nucleic Acids Res. 48, D498-D503 (2020).
44. Korotkevich, G., Sukhov, V. & Sergushichev, A. Fast gene set enrichment analysis. Preprint at doi.org/10.1101/060012 (2016).
45. Yu, G. & He, Q. Y. ReactomePA: An R/Bioconductor package for reactome pathway analysis and visualization. Mol. Biosyst. 12, 477-479 (2016).
46. Street, K. et al. Slingshot: cell lineage and pseudotime inference for single-cell transcriptomics. BMC Genomics 19, 477 (2018).
47. Hafemeister, C. & Satija, R. Normalization and variance stabilization of single-cell RNA-seq data using regularized negative binomial regression. Genome Biol. 20, 296 (2019).
48. La Manno, G. et al. RNA velocity of single cells. Nature 560, 494-498 (2018).
49. Bergen, V., Lange, M., Peidli, S., Wolf, F. A. & Theis, F. J. Generalizing RNA velocity totransient cell states through dynamical modeling. Nat. Biotechnol. 38, 1408-1414 (2020).
50. Van den Berge, K. et al. Trajectory-based differential expression analysis for single-cell sequencing data. Nat. Commun. 11, 1201 (2020).
51. Newman, A. M. et al. Determining cell type abundance and expression from bulk tissues with digital cytometry. Nat. Biotechnol. 37, 773-782 (2019).
52. Patro, R., Duggal, G., Love, M. I., Irizarry, R. A. & Kingsford, C. Salmon provides fast and bias-aware quantification of transcript expression. Nat. Methods 14, 417-419 (2017).
53. Li, H. Aligning sequence reads, clone sequences and assembly contigs with BWA-MEM.Preprint at arxiv.org/abs/1303.3997 (2013).
54. Poplin, R. et al. Scaling accurate genetic variant discovery to tens of thousands of samples. Preprint at doi.org/10.1101/201178 (2017).
55. McLaren, W. et al. The Ensembl Variant Effect Predictor. Genome Biol. 17, 122 (2016).
56. Schneider, C. A., Rasband, W. S. & Eliceiri, K. W. NIH Image to ImageJ: 25 years of image analysis. Nat. Methods 9, 671-675 (2012).
57. Yang, D. et al. The roles of noncardiomyocytes in cardiac remodeling. Int. J. Biol. Sci. 16, 2414-2429 (2020).
58. Fu, X. et al. Specialized fibroblast differentiated states underlie scar formation in the infarcted mouse heart. J. Clin. Invest. 128, 2127-2143 (2018).
59. Lavine, K. J. et al. Distinct macrophage lineages contribute to disparate patterns of cardiac recovery and remodeling in the neonatal and adult heart. Proc. Natl. Acad. Sci. U.S.A. 111, 16029-16034 (2014).
60. Hulsmans, M. et al. Macrophages Facilitate Electrical Conduction in the Heart. Cell 169, 510-522.e20 (2017).
61. Nicolás-Ávila, J. A. et al. A Network of Macrophages Supports Mitochondrial Homeostasis in the Heart. Cell 183, 94-109.e23 (2020).
62. Scruggs, A. M., Grabauskas, G. & Huang, S. K. The role of KCNMB1 and BK channels in myofibroblast differentiation and pulmonary fibrosis. Am. J. Respir. Cell Mol. Biol. 62, 191-203 (2020).
63. Frankenreiter, S. et al. cGMP-Elevating Compounds and Ischemic Conditioning Provide Cardioprotection Against Ischemia and Reperfusion Injury via Cardiomyocyte-Specific BK Channels. Circulation 136, 2337-2355 (2017).
64. Roselli, C. et al. Multi-ethnic genome-wide association study for atrial fibrillation. Nat. Genet. 50, 1225-1233 (2018).
65. Tucker, N. R. et al. Diminished PRRX1 Expression Is Associated with Increased Risk of Atrial Fibrillation and Shortening of the Cardiac Action Potential. Circ. Cardiovasc. Genet. 10, (2017).
66. Yeo, S. Y. et al. A positive feedback loop bi-stably activates fibroblasts. Nat. Commun. 9, (2018).
67. Frankish, A. et al. GENCODE reference annotation for the human and mouse genomes. Nucleic Acids Res. 47, D766-D773 (2019).
68. Alimadadi, A., Munroe, P. B., Joe, B. & Cheng, X. Meta-Analysis of Dilated Cardiomyopathy Using Cardiac RNA-Seq Transcriptomic Datasets. Genes (Basel). 11, 60 (2020).
69. Bae, H. & Lim, I. Effects of nitric oxide on large-conductance Ca²⁺-activated K⁺ currents in human cardiac fibroblasts through PKA and PKG-related pathways. Clin. Exp. Pharmacol. Physiol. 44, 1116-1124 (2017).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### Equivalents and Scope

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

1. A method of diagnosing cardiomyopathy in a subject, comprising:
(a) providing a sample from the subject; and
(b) evaluating the sample for the presence of a population of activated fibroblasts comprising a genetic signature,
wherein the genetic signature comprises increased or decreased expression, relative to a sample provided from a normal subject or a population of normal subjects, of one or more gene products selected from the group consisting of periostin (POSTN), NADPH Oxidase 4 (NOX4), fibroblast activation protein (FAP), collagen type I alpha 1 chain (COL1A1), collagen type I alpha 2 chain (COL1A2), actin alpha 2 (ACTA2), solute carrier family 44 member 5 (SLC44A5), collagen type XXII alpha 1 chain (COL22A1), Ae binding protein 1 (AEBP1), thrombospondin-4 (THBS4), family with sequence similarity 155, member A (FAM155A), teashirt zinc finger homeobox 2 (TSHZ2), juxtaposed with another zinc finger protein 1 (JAZF1), proline and arginine rich end leucine rich repeat protein (PRELP), calsyntenin 2 (CLSTN2), integrin alpha 10 (ITGA10), cell adhesion molecule 1 (CADM1), neuronal growth regulator 1 (NEGR1), platelet-derived growth factor receptor A (PDGFRA), complement component 7 (C7), fibulin 5 (FBLN5), and collagen type IV alpha 4 chain (COL4A4).

2. (canceled)

3. The method of claim 1, wherein the subject is suspected of having or at risk of having cardiomyopathy, or wherein the subject has cardiac disease or a condition predisposing the subject to cardiomyopathy.

4. The method of claim 1, wherein cardiomyopathy is dilated cardiomyopathy or hypertrophic cardiomyopathy.

5. (canceled)

6. The method of claim 1, wherein the step of evaluating the sample comprises performing RNA sequencing on the sample.

7. The method of claim 1, wherein the step of evaluating the sample comprises staining with an antibody.

8. (canceled)

9. The method of claim 1, wherein the genetic signature comprises increased expression, relative to a sample provided from a normal subject or a population of normal subjects, of one or more gene products.

10. The method of claim 9, wherein the one or more gene products is selected from the group consisting of POSTN, NOX4, FAP, COL1A1, COL1A2, ACTA2, SLC44A5, COL22A1, AEBP1, THBS4, FAM155A, JAZF1, PRELP, CLSTN2, ITGA10, CADM1, and TSHZ2.

11. The method of claim 9, wherein the one or more gene products is selected from the group consisting of POSTN, NOX4, FAP, COL1A1, COL1A2, ACTA2, SLC44A5, COL22A1, AEBP1, THBS4, FAM155A, and TSHZ2.

12. The method of claim 9, wherein the one or more gene products is selected from the group consisting of POSTN, COL22A1, and THBS4.

13. The method of claim 1, wherein the genetic signature comprises decreased expression, relative to a sample provided from a normal subject or a population of normal subjects, of one or more gene products.

14. The method of claim 13, wherein the one or more gene products are selected from the group consisting of NEGR1, PDGFRA, C7, FBLN5, and COL4A4.

15. The method of claim 1, wherein the genetic signature comprises increased or decreased expression, relative to a sample provided from a normal subject or a population of normal subjects, of one or more gene products, wherein one of the gene products is COL22A1.

16. The method of claim 1, wherein the genetic signature comprises increased or decreased expression, relative to a sample provided from a normal subject or a population of normal subjects, of two or more gene products, wherein one of the gene products is COL22A1.

17. (canceled)

18. The method of claim 1, wherein the genetic signature comprises increased or decreased expression, relative to a sample provided from a normal subject or a population of normal subjects, of one or more gene products, wherein one of the gene products is POSTN.

19-20. (canceled)

21. The method of claim 1, wherein the genetic signature comprises increased or decreased expression, relative to a sample provided from a normal subject or a population of normal subjects, of one or more gene products, wherein one of the gene products is THBS4.

22-23. (canceled)

24. The method of claim 1, wherein the genetic signature comprises increased or decreased expression of one or more genes selected from the group consisting of NEGR1, FBLN5, PRELP, CLSTN2, ITGA10, JAZF1, COL22A1, AEBP1, FGF14, THBS4, FAP, and CADM1.

25. A method of treating cardiomyopathy in a subject in need thereof, the method comprising administering to the subject a treatment based on the presence of a population of activated fibroblasts comprising a genetic signature, wherein the genetic signature comprises increased or decreased expression, relative to a sample provided from a normal subject or a population of normal subjects, of one or more gene products selected from the group consisting of periostin (POSTN), NADPH Oxidase 4 (NOX4), fibroblast activation protein (FAP), collagen type I alpha 1 chain COL1A1, collagen type I alpha 2 chain (COL1A2), actin alpha 2 (ACTA2), solute carrier family 44 member 5 (SLC44A5), collagen type XXII alpha 1 chain (COL22A1), Ae binding protein 1 (AEBP1), thrombospondin-4 (THBS4), family with sequence similarity 155, member A (FAM155A), teashirt zinc finger homeobox 2 (TSHZ2), juxtaposed with another zinc finger protein 1 (JAZF1), proline and arginine rich end leucine rich repeat protein (PRELP), calsyntenin 2 (CLSTN2), integrin alpha 10 (ITGA10), cell adhesion molecule 1 (CADM1), neuronal growth regulator 1 (NEGR1), platelet-derived growth factor receptor A (PDGFRA), complement component 7 (C7), fibulin 5 (FBLN5), and collagen type IV alpha 4 chain (COL4A4).

26-29. (canceled)

30. The method of claim 25, wherein the treatment comprises exercise, surgery, use of a medical device, and/or pharmacological intervention.

31. The method of claim 30, wherein the treatment comprises use of a pacemaker, use of a defibrillator, use of a ventricular assist device, ablation, an angiotensin-converting enzyme (ACE) inhibitor, an angiotensin II receptor blocker, a beta blocker, a diuretic, digoxin, a blood-thinning medication, or a heart transplant.

32-50. (canceled)

51. A method of modulating expression of one or more gene products associated with activated cardiac fibroblasts in a subject in need thereof, the method comprising administering to the subject an agent capable of modulating the activity of one or more gene products associated with activation of cardiac fibroblasts, wherein the one or more gene products are selected from the group consisting of periostin (POSTN), NADPH Oxidase 4 (NOX4), fibroblast activation protein (FAP), collagen type I alpha 1 chain COL1A1, collagen type I alpha 2 chain (COL1A2), actin alpha 2 (ACTA2), solute carrier family 44 member 5 (SLC44A5), collagen type XXII alpha 1 chain (COL22A1), Ae binding protein 1 (AEBP1), thrombospondin-4 (THBS4), family with sequence similarity 155, member A (FAM155A), teashirt zinc finger homeobox 2 (TSHZ2), juxtaposed with another zinc finger protein 1 (JAZF1), proline and arginine rich end leucine rich repeat protein (PRELP), calsyntenin 2 (CLSTN2), integrin alpha 10 (ITGA10), cell adhesion molecule 1 (CADM1), neuronal growth regulator 1 (NEGR1), platelet-derived growth factor receptor A (PDGFRA), complement component 7 (C7), fibulin 5 (FBLN5), and collagen type IV alpha 4 chain (COL4A4).

52-86. (canceled)
